(19) United States
(12) Patent Application Publication
(10) Pub. No.: US 2025/0270505 A1
(43) Pub. Date: Aug. 28, 2025

(54) METHOD AND SYSTEM FOR PRENATAL AND NEONATAL DIAGNOSIS OF PSYCHIATRIC DISORDERS

(71) Applicant: ITAYANDBIOND LTD, Tel Aviv-Yaffo (IL)

(72) Inventors: Nisim PERETS, Tel Aviv (IL); Nir WAISKOPF, Jerusalem (IL); Naresh MUTUKULA, Jerusalem (IL); Jonatan DARR, Holon (IL); Shmulik BEZALEL, Limassol (CY)

(21) Appl. No.: 19/200,492

(22) Filed: May 6, 2025

Related U.S. Application Data
(63) Continuation of application No. PCT/IL2023/051157, filed on Nov. 9, 2023.
(60) Provisional application No. 63/423,892, filed on Nov. 9, 2022.
(60) Provisional application No. 63/423,894, filed on Nov. 9, 2022.

(51) Int. Cl. C12N 5/0793; A61B 5/16; G01N 33/483

(52) U.S. Cl. CPC C12N 5/0619; A61B 5/16; G01N 33/4836; A61B 2503/02; A61B 2503/045; C12N 2503/04; C12N 2513/00

(57) ABSTRACT

Disclosed are method and systems for diagnosis of a psychiatric disorder (PD) in prenatal or neonatal derived-brain organoids by determining a signal and/or attribute.

### CROSS REFERENCE TO RELATED APPLICATIONS

This application is a Bypass Continuation of PCT Patent Application No. PCT/IL2023/051157 having International filing date of Nov. 9, 2023, which claims the benefit of priority of U.S. Provisional Patent Application No. 63/423,892, filed Nov. 9, 2022, and U.S. Provisional Patent Application No. 63/423,894, filed Nov. 9, 2022, the contents of which are all incorporated herein by reference in their entirety.

### TECHNICAL FIELD

The present disclosure generally relates to assessment of a psychiatric disorder (PD) at early developmental stages, namely, prenatal diagnosis. More specifically, the invention relates to prenatal diagnosis of PD using brain organoids.

### BACKGROUND OF THE INVENTION

Psychiatric disorders (PD) have genetic bases that may determine the course of development of the disorder (i.e., genetic PD), yet in some manifestations, PD involves a strong influence of non-genetic, environmental factors (and epigenetic) that impact the risk and contribute to the development of the disorder and its symptoms (i.e., non-genetic PD).

The pathogenesis of at least non-genetic PD is considered spontaneous and unknown (idiopathic) with respect to the timing of the development of the disorder, and to the underlying mechanism. Accordingly, experimental research of non-genetic PD predicts the involvement and influence of more than several hundred different genes and factors in the development of these disorders and assumes a very poor understanding of the contribution of environmental factors to the interplay with the genetic factors. Non-genetic PD is therefore considered as multifactorial and complex.

PD affects mental, emotional and/or behavioral aspects that impair or diminish the function of the individual. PD, with emphasis on neurodevelopmental disorders such as Attention Deficit Hyperactivity Disorder (ADHD/ADD), or Bipolar disorder, deeply impact life quality, notwithstanding the relative manageability of these disorders using psychoactive or anticonvulsant medications.

Other neurodevelopmental disorders, such as Autism Spectrum Disorders (ASD) lack pharmaceutical solutions.

Perturbed cognitive functionalities associated with PD include, but are not limited to: cognitive impairment/rigidity (e.g., adaptive learning), social problems (e.g., communication and social interaction), repetitive and restricted patterns of behavior, motivation, and/or attention.

The complex etiology and genetic bases of PD especially of non-genetic PD, means that diagnosis of PD using molecular genetic tools is less feasible, and practically restricts diagnosis and evaluation of PD severity to clinical signs, relying on symptoms, phenotypic behavior, and disturbances of mood or psychosis. Therefore, currently, the process of PD diagnosis requires the presence, involvement, and preferably the cooperation of the patient through a series of sessions and tasks that may be laborious and exhausting, as well as qualitative and subjective to some extent and is only possible when the subject reaches childhood, in some cases only in adolescence, or later.

For the school and families, the burden is high, and at least some parents may have considered abortion had the condition been diagnosed early in the pregnancy.

In addition, due to the ‘late’ clinical diagnosis, the effect of medication, if given, in the prenatal stages of human embryonic development remains unknown. In this regard, earlier diagnosis holds the potential of ameliorating or alleviating PD.

There is therefore an urgent need for early diagnosis of PD, preferably at neonatal stage.

Brain organoids are 3D-cultured cell aggregates, derived from pluripotent stem cells (PSCs) including induced pluripotent stem cells (iPSC) that can recapitulate the structure and function of different brain regions, including high-order brain regions involved in cognition and learning, such as the cerebral cortex. Brain organoids are useful in modeling neural circuits and investigating neurodevelopmental disorders, psychiatry, and psychiatric drugs.

Since contributions of genes and environment co-exist in PD, assumingly as early as the beginning of embryonic development, providing prenatal or early neonatal assessment for PD is of high importance.

### SUMMARY OF THE INVENTION

According to some aspects, the disclosure provides methods and systems for prenatal or neonatal assessment of a psychiatric disorder (PD) utilizing brain organoids.

The herein disclosed methods include generating brain organoids from cells of an embryo, a fetus, or a newborn, and determine a signal and/or attribute to thereby identify the organoid as “PD-derived” or as derived from healthy cells.

Advantageously, in some embodiments, it is disclosed that the signal and/or attribute includes structural phenotypes, some accessible by visualization of the brain organoid using standard contrast illumination methods, others by simple fluorescence imaging of the organoid's projection. It was surprisingly found that PD-derived organoids are distinguished from healthy organoids in visual attributes related to structure, shape and size, and that some culturing conditions further promote those differences between them.

Further advantageous, is the use of conditional and reinforcement learning on neonatal or prenatal derived organoids, for driving computational simulation based on the neural network response to stimuli sessions provided to the organoid. The electrical activity signal recorded from prenatal or neonatal organoids during functional neuro-computational stimuli-response assay, is indicative of synaptic plasticity and neuronal coding and is used in this disclosure as a type of cognitive simulation to determine a behavior, including a learning-behavior, of the prenatal or neonatal derived organoid.

Also provided are AI algorithms, including for classifying the brain organoid based on a degree of similarity, and methods for training a machine learning algorithm to determine brain organoids behavior.

Reference is made to FIG. 1.

The herein disclosed system facilitates assessment of the abovementioned visual and electric signals by offering an advantageous interface for the interplay between a processor, a prenatal or neonatal 3D organoid or 2D neuronal culture thereof, and a sensor, and optionally a source of stimuli.

The system provides a platform for PD drug discovery and/or for personalized medicine as a predictive tool for the clinical success of treatment with a medicament for PD a psychiatric, neurologic, neurodevelopmental and/or neurodegenerative condition, thereby promoting sub-populations of PD patients and promoting biomarkers discovery by complemental biochemical evaluations.

Reference is made to FIG. 7A.

According to some aspects the invention provides a method for prenatal and/or neonatal assessment of psychiatric disorder (PD), the method comprising:
(i) obtaining human prenatal or neonatal cells; (ii) generating a prenatal or neonatal derived-brain organoid; and (iii) assessing one or more signal and/or attribute of the brain organoid. Each possibility is a separate embodiment.

According to some embodiments, the assessing comprises determining a degree of similarity between the one or more signal(s) and/or attribute(s) of the prenatal or neonatal brain organoid and PD-derived organoids, or between the one or more signal(s) and/or attribute(s) of the prenatal or neonatal brain organoid and healthy organoids. Each possibility is a separate embodiment.

In some additional embodiments, the method comprises classifying the prenatal or neonatal organoid as ‘PD’ or ‘healthy’ based on the determined degree of similarity. Each possibility is a separate embodiment.

In some further additional embodiments, the method further comprise scoring or classifying the prenatal or neonatal brain organoid according to ‘severity of PD’ based on the determined degree of similarity. Each possibility is a separate embodiment.

According to some embodiments, the PD comprises non-genetic psychiatric disorder (non-genetic PD).

In some specific embodiments, the PD is selected from one or more disease selected from of Autism Spectrum Disorders (ASD), Bipolar disorder, Attention Deficit Hyperactivity Disorder (ADHD/ADD), Schizophrenia, Depression, Obsessive-Compulsive Disorders (OCD), Rett syndrome, Fragile X Syndrome, Intellectual Developmental Disorder, Down Syndrome, Williams Syndrome, Prader-Willi Syndrome, Angelman Syndrome, Smith-Magenis Syndrome, Epilepsy, Parkinson's disease, and Alzheimer's disease, or any combination thereof. Each possibility is a separate embodiment.

In some further specific embodiments, the PD comprises an Autistic Spectrum Disorder (ASD).

In some further specific embodiments, the ASD comprises non-syndromic idiopathic ASD.

According to some embodiments, the prenatal or neonatal cells are obtained from one or more of an embryo, a fetus, and a newborn, or any combination thereof. Each possibility is a separate embodiment.

According to some specific embodiments, the prenatal or neonatal cells are obtained by one or more of Chorionic Villus Sampling (CVS), amniotic fluid test (Amniocentesis), in-vitro fertilization (IVF), cord blood collection, and/or biopsies, or any combination thereof. Each possibility is a separate embodiment.

In some specific embodiments, the prenatal or neonatal cells include Human Amniotic Epithelial Cells (HAEpiC). Each possibility is a separate embodiment.

According to some embodiments, generating a prenatal or neonatal derived-brain organoid comprises reprogramming the obtained prenatal or neonatal cells to induced pluripotent stem cells (hiPSC). Each possibility is a separate embodiment.

In some embodiments, generating a prenatal or neonatal derived-brain organoid comprises generating Neural Progenitor Cells (NPC) from the iPSC. Each possibility is a separate embodiment.

In some embodiments, generating a prenatal and/or neonatal derived-brain organoid comprises further differentiation of NPC to neurons. Each possibility is a separate embodiment.

In some embodiments, generating a prenatal or neonatal derived-brain organoid includes generating neurons directly from the iPSCs. Each possibility is a separate embodiment.

In some related embodiments, the prenatal and/or neonatal brain organoid is selected from one or more of a midbrain organoid, hippocampal organoid, striatal organoid, neocortical organoid, cerebral organoid and/or cortical organoid, or any combination thereof. Each possibility is a separate embodiment.

In some related embodiments, the prenatal and/or neonatal brain organoid comprises cerebral organoid and/or cortical organoid. Each possibility is a separate embodiment.

In some related embodiments, the prenatal and/or neonatal brain organoid comprises neural vesicle/rosette structures comprising neural stem cells around ventricles, and neurons surrounding the neural stem cells. Each possibility is a separate embodiment.

According to embodiments, the one or more signal(s) and/or attribute(s) comprise a visual 3D structure and/or an electrical activity of the organoid. Each possibility is a separate embodiment.

According to specific embodiments, the one or more attribute(s) comprise one or more of organoid shape, organoid size, spines density, or any combination thereof. Each possibility is a separate embodiment.

According to embodiments, the one or more attribute(s) comprise organoid shape and/or size. Each possibility is a separate embodiment.

In some embodiment, the attribute of organoid shape comprises a deviation in the amount of neural projections of a PD-derived organoid from a the amount of neural projections of a healthy organoid; wherein the deviation includes increment and/or decrease in the amount of sprouting. Each possibility is a separate embodiment.

In some embodiment, the attribute of organoid size comprises a deviation in a mean organoid diameter (A) of a PD-derived organoid from a mean organoid diameter of a healthy organoid; wherein a deviation includes increment and/or decrease in the diameter. Each possibility is a separate embodiment.

In some embodiment, a mean organoid diameter (A) of a PD-derived organoid deviates by at least about 25 μm from a mean organoid diameter of a healthy organoid; wherein a deviation includes increment and/or decrease in the diameter. Each possibility is a separate embodiment.

In some embodiment, wherein the attribute of organoid size includes a deviation in a mean ratio between the organoid diameter (A) (μm) and its outwards neural projection (B) (μm) of a PD-derived organoid from a mean ratio of a healthy organoid; wherein the deviation includes increment and/or decrease in the ratio. Each possibility is a separate embodiment.

In some embodiment, a mean organoid ratio between the organoid diameter (A) (μm) and its outwards neural projection (B) (μm) of a PD-derived organoid deviates by at least about 20% from a mean ratio of a healthy organoid; wherein a deviation includes increment and/or decrease in the diameter. Each possibility is a separate embodiment.

In some embodiments, the assessing of the attribute of organoid shape and/or size comprises incubating the organoid with an activator or an inhibitor before determining organoid diameter (A) (μm). Each possibility is a separate embodiment.

In some embodiments, the attribute of spines density comprises a deviation in an amount/number of spines along a dendritic projection of a PD-derived organoid from an amount/number of spines along a dendritic projection of healthy organoid, wherein the deviation includes increment and/or decrease in the density.

According to other embodiments, the one or more signal(s) comprises electrical activity; and wherein the electrical activity is indicative of neuronal function. Each possibility is a separate possibility.

In some embodiments, the electrical activity comprises electrophysiological measurements and/or activity reporter imaging. Each possibility is a separate possibility.

In some embodiments, the electrical activity indicative of neuronal function comprises electrical activity signal recorded from the brain organoid in response to one or more treatment/stimuli sessions, each session comprise electrophysiological stimuli provided to the brain organoid. Each possibility is a separate embodiment.

According to some embodiments, the assessing of the one or more signal(s) of electrical activity comprises determining a behavior of the brain organoid based on the electrical activity recorded in response to the one or more treatment/stimuli session(s). Each possibility is a separate embodiment.

In some embodiments, the assessing comprises determining a degree of similarity between the determined behavior of the prenatal or neonatal brain organoid and behavior of PD-derived organoids, or between the determined behavior of the prenatal or neonatal brain organoid and behavior of a healthy organoid. Each possibility is a separate embodiment.

In some specific embodiments, the treatment/stimulus provided to the brain organoid in the one or more session(s) are fixed/predetermined (open loop). Each possibility is a separate embodiment.

In some embodiments, the AI algorithm is trained on brain-organoids behaviors of a plurality of healthy and/or PD derived brain organoids in response to the predetermined treatment/stimulus, wherein the training data is labeled according to one or more predetermined parameters of the treatment/stimulus (open loop). Each possibility is a separate embodiment.

According to some embodiments, the AI algorithm is continuously reinforced, based on the determined brain-organoid behavior, to thereby improve the predicted behavior (open and closed loop).

In some specific embodiments, the stimulus provided to the brain organoid is determined according to the determined brain-organoid behavior. (closed loop)

In some specific embodiments, the electrophysiological stimulus provided to the brain organoid in a latter session is determined based on the brain organoids' behavior determined in response to one or more former stimuli sessions (closed loop). Each possibility is a separate embodiment.

In a further specific embodiment, the electrophysiological stimulus provided to the brain organoid is positive or negative feedback stimuli. Each possibility is a separate embodiment.

In some embodiments, a change in the brain (closed loop) organoids behavior between a former and the latter sessions is indicative of a learning-behavior response of the brain organoid (closed loop).

According to some embodiments, the AI algorithm is trained on brain organoids behaviors of a plurality of healthy and/or PD derived brain organoids, wherein the training data is labeled according to one or more parameters of the elastic stimuli/positive or negative treatment/stimulus (closed loop).

In some embodiments, the method further comprise repeatedly providing stimuli sessions to the organoid, obtaining data recorded in response to each session, and determining the brain-organoid behavior based on the recorded data, after treatment of the organoid with a PD medicament including neurological, and/or neurodevelopmental medicaments.

According to some embodiments, the obtained brain organoid comprises 3D brain organoid in culture.

According to some related embodiments, the obtained brain organoid comprises tissue and/or cells thereof in 2D culture, and wherein the tissue and/or cells comprise sliced tissue and/or dissociated cells resulted from any of enzymatic, chemical, and/or mechanical processing of a formed 3D brain organoid. Each possibility is a separate embodiment.

In some embodiments, the brain organoid is an undetermined brain organoid having unknown severity of PD.

According to some embodiments, the assessment of one or more signal(s) and/or attribute(s) further comprises assessing gene expression and genomic abnormalities. Each possibility is a separate embodiment.

According to another aspect, the invention provides a system for prenatal and/or neonatal assessment of psychiatric disorder (PD), the system comprising:
(i) a prenatal or neonatal-derived brain organoid; (ii) one or more sensor(s) coupled to a recorder capable of detecting and archiving/recording one or more signal(s) and/or attribute(s) of the prenatal or neonatal-derived brain organoid;
(iii) a micro-controller unit (MCU) configured to receive, integrate and/or transmit information/data of the one or more signal(s) and/or attribute(s); (iv) processor configured to: (a) obtain the recorded data of the one or more signal(s) and/or attribute(s), and (b) assessing the one or more signal(s) and/or attribute(s) based on the data.

In some embodiments, the assessing comprises determining a degree of similarity between the one or more signal(s) and/or attribute(s) of the prenatal or neonatal brain organoid and PD-derived organoids, or between the one or more signal(s) and/or attribute(s) of the prenatal or neonatal brain organoid and healthy organoids. Each possibility is a separate embodiment.

In some embodiments, the processor is configured to classify the prenatal or neonatal organoid as ‘PD’ or ‘healthy’ based on the degree of similarity.

In some related embodiments, the computer/processor is further configured to: apply an AI algorithm on the data of the one or more signal(s) and/or attribute(s) to thereby classify the brain organoid based on a degree of similarity of the one or more signal(s) and/or attribute(s) of the organoid to PD-derived organoids or healthy organoids; and wherein the algorithm classifies the organoid according to ‘severity of PD’ or ‘healthy’. Each possibility is a separate embodiment.

According to some embodiments, the one or more signal(s) and/or attribute(s) comprise a visual external phenotype of the organoid and/or an electrophysiological activity. Each possibility is a separate embodiment.

In some embodiments, the one or more attribute(s) comprises one or more of organoid shape, organoid size, and spines density, or any combination thereof. Each possibility is a separate embodiment.

In some embodiments, the attribute of organoid shape comprises a deviation in the amount of neural projections of a PD-derived organoid from a the amount of neural projections of a healthy organoid; wherein the deviation includes increment and/or decrease in the amount of sprouting. Each possibility is a separate embodiment.

In some embodiments, the attribute of organoid size comprises a deviation in a mean organoid diameter (A) of a PD-derived organoid from a mean organoid diameter (A) of a healthy organoid; wherein the deviation includes increment and/or decrease in the diameter. Each possibility is a separate embodiment.

In some embodiments, the attribute of organoid size includes a deviation in a ratio between the organoid diameter (A) (μm) and its outwards neural projection (B) (μm) of a PD-derived organoid from a ratio organoid diameter (A) of a healthy organoid; wherein the deviation includes increment and/or decrease in the ratio. Each possibility is a separate embodiment.

In some embodiments, assessing the attribute of organoid shape and/or size comprises incubating the organoid with an activator or an inhibitor before determining organoid diameter (A) (μm). Each possibility is a separate embodiment.

In some embodiments, the attribute of spines density comprises a deviation in an amount/number of spines along a dendritic projection of a PD-derived organoid from an amount of spines along a dendritic projection of healthy organoid, wherein the deviation includes increment and/or decrease in the density. Each possibility is a separate embodiment.

According to some embodiments, the one or more signal(s) comprises electrical activity; and wherein the electrical activity is indicative of neuronal function.

In some specific embodiments, the electrical activity comprises electrophysiological measurements and/or activity reporter imaging. Each possibility is a separate embodiment.

According to some embodiments, the system further comprises a stimuli system capable of delivering stimuli to the prenatal or neonatal brain organoid; and wherein the processor is further configured to send instructions to the stimuli system to provide one or more treatment/stimuli sessions, each session comprises treatment/electrophysiological stimuli provided to the brain organoid.

In some embodiments, the sensor comprises a multi-electrode array (MEA) coupled to one or more recording head stage (RHS) capable of recording electrical activity from the brain organoid.

In some embodiments, the electrical activity indicative of neuronal function comprises electrical activity signal recorded from the brain organoid in response to the one or more treatment/stimuli session(s) comprising the electrophysiological stimuli provided to the brain organoid.

In some embodiments, the computer/processor is further configured to determine a behavior of the brain organoid based on the electrical activity signal recorded in response to the one or more treatment/stimuli session(s).

In some embodiments, the computer/processor is further configured to apply an AI algorithm on the brain organoid behavior to thereby classify the brain organoid based on a degree of similarity of the determined behavior of the brain organoid to a predicted behavior of a PD-derived brain organoids and/or heathy organoids; and wherein the algorithm classifies the organoid according to ‘severity of PD’ or ‘healthy’.

In some embodiments, the treatment/stimulus provided to the brain organoid in the one or more session(s) are fixed/predetermined. Each possibility is a separate embodiment. (open loop)

In some embodiments, the treatment/stimulus provided to the brain organoid in a latter session is determined based on the brain organoid behavior determined in response to one or more former stimuli sessions. Each possibility is a separate embodiment. (closed loop)

In some embodiments, the treatment/stimulus provided to the brain organoid is positive or negative feedback stimuli; and wherein a change in the brain-organoids behavior between a former and the latter sessions is indicative of a learning behavior response of the brain organoid. Each possibility is a separate embodiment. (closed loop)

In some embodiments, the classifying of the brain organoid is based on a degree of similarity of the learning-behavior response to a predicted learning-behavior response of a PD-derived brain organoid and/or of a heathy organoid (closed loop). Each possibility is a separate embodiment.

According to some further embodiments, the processor is configured to generate a visual simulation representative of the determined organoid behavior.

In some embodiments, the visual simulation comprises a computer game.

In some embodiments, the computer game is configured to present the evaluation of functionality of neuronal network selected from one or more of: memory, cognitive rigidity, motivation, repetitive behavior, attention, social interaction and/or facial expression, or any combination thereof. Each possibility is a separate embodiment.

In some embodiments, the processor is further configured to repeatedly providing stimuli sessions to the organoid, obtaining data recorded in response to each session, and determining the brain-organoid behavior based on the recorded data, after treatment of the organoid with a PD medicament including neurological and/or neurodevelopmental medicaments. Each possibility is a separate embodiment.

In some embodiments, the processor is further configured to determine the efficacy of the treatment.

According to some embodiments, the obtained brain organoid comprises 3D brain organoid in culture.

According to some embodiments, the obtained brain organoid comprises tissue and/or cells thereof in 2D culture, and wherein the tissue and/or cells comprise sliced tissue and/or dissociated cells resulted from any of enzymatic, chemical, and/or mechanical processing of a 3D brain organoid. Each possibility is a separate embodiment.

In some embodiments, the brain organoid is an undetermined brain organoid having unknown severity of PD.

According to an additional aspect, the invention provides a prenatal or neonatal brain organoid for assessment of a psychiatric disorder (PD) wherein the organoid is generated from human prenatal or neonatal cells, and wherein the assessment comprises assessment of one or more signal(s) and/or attribute(s).

According to some embodiments, the PD comprises one or more of Autism Spectrum Disorders (ASD), Bipolar disorder, Attention Deficit Hyperactivity Disorder (ADHD/ADD), Schizophrenia, Depression, Obsessive-Compulsive Disorders (OCD), Epilepsy, Rett syndrome, Fragile X Syndrome, Intellectual Developmental Disorder, Down Syndrome, Williams Syndrome, Prader-Willi Syndrome, Angelman Syndrome, Smith-Magenis Syndrome, Parkinson's disease, and Alzheimer's disease, or any combination thereof. Each possibility is a separate embodiment.

In some embodiments, the PD comprises non-genetic psychiatric disorder (non-genetic PD).

In some embodiments, the assessing of one or more signal and/or attribute is with respect to healthy organoids.

In some embodiments, the one or more signal(s) and/or attribute(s) comprise a visual 3D structure and/or an electrophysiological activity of the organoid of the organoid.

In some embodiments, the one or more attribute(s) comprises one or more of organoid shape, organoid size, and spines density, or any combination thereof.

In some embodiments, the one or more signal(s) comprises electrical activity; and wherein the electrical activity is indicative of neuronal function recorded from the brain organoid in response to one or more treatment/stimuli.

In some embodiments, the treatment/stimuli comprise electrophysiological stimuli provided to the brain organoid.

In some embodiments, the one or more attribute(s) comprises organoid behavior determined based on electrical activity recorded from the brain organoid in response to one or more treatment/stimuli.

According to some embodiments, the invention provides a method for training an AI algorithm for determining organoids behavior, the method comprising:
obtaining a plurality of PD-derived brain organoid and a plurality of healthy brain organoid; (b) providing one or more stimuli sessions, each session comprising a stimuli provided to the brain organoid; (c) obtaining data recorded in response to the one or more treatment/stimuli session(s), the data is indicative of neuronal function/activity of the brain organoid; (d) labeling the data according to parameters of the one or more stimuli session(s) and associating the labeled data with the PD-derived brain organoid and/or with the plurality of healthy brain organoid; (e) applying an AI algorithm on the data to learn patterns and relationships and to adjust parameters of a model for organoid behavior prediction; thereby training the algorithm for determining a brain-organoids behavior based on the data recorded in response to the one or more treatment/stimuli session(s).

In some embodiments, the AI algorithm is further trained to classify the organoids plurality of PD-derived brain organoids and/or healthy organoids based on the determined organoids' behavior as having ‘PD-derived behavior’ or a ‘heathy behavior’; thereby classifying the brain organoids based on a degree of similarity of their determined behavior to a predicted behavior of a PD-derived brain organoid and/or a heathy organoid.

In some embodiments, the obtaining of PD-derived brain organoid comprises organoids having a range of PD severities, and wherein the association of the labeled data with the PD-derived brain organoid comprises associating the labeled data with the range of PD severities; thereby augmenting the prediction behavior model to include a range of severities.

In some embodiments, the data indicative of neuronal function/activity of the brain organoid is divided to a ‘training dataset’ and ‘validation set’, and wherein the ‘validation set’ comprises unlabeled data used to improve model performance.

In some embodiments, the AI algorithm includes supervised learning, unsupervised learning, semi-supervised learning, and reinforced learning, or any combination thereof. Each possibility is a separate embodiment.

In some embodiments, the AI algorithm includes a supervised machine learning algorithm capable of regression and/or classification, including for example, but not limited to one or more of: Support-vector machines, Linear regression, Logistic regression, Naive Bayes, Linear discriminant analysis, Decision trees, K-nearest neighbor algorithm, Neural networks (Multilayer perceptron), Similarity learning, or any combination thereof. Each possibility is a separate embodiment.

Other non-limiting examples of supervised machine learning algorithm capable of regression and/or classification, including for example, but not limited to: Analytical learning, Artificial neural network, Backpropagation, Boosting (meta-algorithm), Bayesian statistics, Case-based reasoning, Decision tree learning, Inductive logic programming, Gaussian process regression, Genetic, programming, Group method of data handling, Kernel estimators, Learning automata, Learning classifier systems, Learning vector quantization, Minimum message length (decision trees, decision graphs, etc.), Multilinear subspace learning, Naive Bayes classifier, Maximum entropy classifier, Conditional random field, Nearest neighbor algorithm, Probably approximately correct learning (PAC) learning, Ripple down rules, a knowledge acquisition methodology, Symbolic machine learning algorithms, Subsymbolic machine learning algorithms, Support vector machines, Minimum complexity machines (MCM), Random forests, Ensembles of classifiers, Ordinal classification, Data pre-processing, Handling imbalanced datasets, Statistical relational learning, Proaftn, a multicriteria classification algorithm.

In some embodiments, the training comprises open loop training mode, wherein the treatments/stimuli provided to the brain organoid in the one or more session(s) are predetermined.

In some embodiments, the training comprises closed loop training mode, wherein the treatments/stimuli provided to the brain organoid in the one or more session(s) is determined according to the determined brain-organoid behavior.

In some embodiments, at least the plurality of healthy brain organoids is generated from prenatal or neonatal cells.

In some embodiments, at least the plurality of PD-derived brain organoid is generated from prenatal or neonatal cells.

In some embodiments, the training of the AI algorithm for determining organoids behavior includes determining prenatal and/or neonatal organoids behavior.

Further advantageous is the herein-disclosed assessment of a biological or a physical attribute that is surprisingly associated with a psychiatric disorder (PD)-derived brain organoid. The biological or physical attribute is assessed by a change/difference in one or more signal(s) and/or attribute(s) of a prenatal or neonatal derived-brain organoid, and/or tissue and/or cells thereof.

According to some aspects, the method for prenatal and/or neonatal assessment of psychiatric disorder (PD) comprises (i) obtaining human prenatal or neonatal cells; (ii) generating a prenatal or neonatal derived-brain organoid from the human prenatal or neonatal cells; and (iii) assessing one or more signal(s) and/or attribute(s) of the brain organoid.

In some embodiments, PD comprises non-genetic psychiatric disorder (non-genetic PD).

In some embodiments, the method further comprises a step of obtaining PD cells and generating a PD-derived brain organoid.

In some embodiments, the PD comprises one or more of Autism Spectrum Disorders (ASD), Bipolar disorder, Attention Deficit Hyperactivity Disorder (ADHD/ADD), Schizophrenia, Depression, Obsessive-Compulsive Disorders (OCD), Epilepsy, or any combination thereof.

In some embodiments, the PD comprises an Autistic Spectrum Disorder (ASD).

In some embodiments, the ASD comprises non-syndromic idiopathic ASD.

In some embodiments, the human prenatal or neonatal cells are obtained from an embryo, a fetus, or a newborn at his early neonatal stage.

In some embodiments, the human prenatal or neonatal cells are obtained by Chorionic Villus Sampling (CVS), amniotic fluid test (Amniocentesis), in-vitro fertilization (IVF), cord blood collection, and/or biopsies.

In some embodiments, the obtained human prenatal or neonatal cells comprise tissue stem cells.

In some embodiments, the obtained human cells are transformed to pluripotent stem cells (hiPSC).

In some embodiments, the obtained human cells are transformed to iPSC-derived Neural Progenitor Cells (NPC).

In some embodiments, the NPC are further differentiated.

In some embodiments, the brain organoid comprises at least a cerebral and/or cortical tissue.

In some embodiments, the assessment of PD comprises assessing a change/difference in one or more signal(s) and/or attribute(s) of the prenatal or neonatal derived-brain organoid, compared with a healthy brain organoid.

In some embodiments, the assessment of the one or more signal(s) and/or attribute(s) of the brain organoid comprises assessing cells and/or tissue.

In some embodiments, the assessment of the one or more signal(s) and/or attribute(s) of the brain organoid comprises assessing physical and/or biological attribute of the brain organoid; In some embodiments, the physical and/or biological attribute are indicated by optic/light signal or electric signal.

In some embodiments, the optic/light signal or electric signal comprises information indicative of physical and/or biological attributes of the brain organoid, the cells and/or tissue thereof.

In some embodiments, the physical and/or biological attribute indicated by the optic/light signal comprises external phenotype, appearance/morphology, 2D/3D structure, shape, size, and texture, or any combination thereof.

In some embodiments, the physical and/or biological attribute indicated by the optic/light signal comprises the size of the brain organoid and/or the length of neural projection thereof.

In some embodiments, the physical and/or biological attribute indicated by the optic/light signal further comprises spontaneous or induced neuronal function/activity, gene expression, and genomic abnormalities/structure, or any combination thereof.

In some embodiments, the physical and/or biological attribute indicated by the electric signal comprises information indicative of spontaneous or induced neuronal function/activity.

In some embodiments, prior to the assessment of PD a pre-treatment is applied to the prenatal or neonatal derived-brain organoid, or to a cell obtained, transformed, or differentiated to generate the prenatal or neonatal derived-brain organoid.

In some embodiments, the pre-treatment comprises one or more of altered gene expression, expression of a genetic reporter, or exposure to electric pulse, optic/light signal, heat, a chemical agent/drug, starvation, or hypoxia, or any combination thereof.

In some embodiments, the assessment of PD further comprises computational bioinformatic data derived from gene expression or genetic examination of the prenatal or neonatal derived-brain organoid.

In some embodiments, the assessment of PD further comprises assessing a learning-behavior response of the prenatal or neonatal derived-brain organoid.

According to some aspects, the system for prenatal and/or neonatal assessment of psychiatric disorder (PD) comprises: (i) a prenatal or neonatal-derived brain organoid and a PD-derived brain organoid; (ii) a sensor coupled to a recorder capable of detecting and archiving one or more signal(s) and/or attribute(s) of the prenatal or neonatal-derived brain organoid; (iii) at least one micro-controller unit (MCU) configured to integrate and/or transmit information derived from the one or more signal(s) and/or attribute(s); (iv) a computer/processor configured to apply a machine learning algorithm to determine a probability or range of probability of the prenatal-derived brain organoid being derived from PD cells/subject.

In some embodiments, the computer/processor obtains input data from a plurality of prenatal or neonatal-derived brain organoids and from a plurality of PD-derived brain organoids; In some embodiments, the input data comprises a plurality of information derived from the one or more signal(s) and/or attribute(s).

In some embodiments, the system further comprises a brain organoid derived from cells of healthy subjects.

In some embodiments, the sensor is coupled to a recorder; In some embodiments, the sensor coupled to a recorder is capable of detecting and archiving.

In some embodiments, the sensor comprises an imaging device coupled to a camera;

In some embodiments, the at least one signal and/or attribute detected and archived is optic/light signal; In some embodiments, the optic/light signal comprises information indicative of physical and/or biological attributes of the prenatal or neonatal-derived brain organoid.

In some embodiments, the physical and/or biological attribute indicated by the optic/light signal comprises external phenotype, appearance/morphology, 2D/3D structure, shape, size, and texture, or any combination thereof.

In some embodiments, the physical and/or biological attribute indicated by the optic/light signal further comprises, gene expression, and genomic abnormalities/structure, or any combination thereof;

In some embodiments, the information indicative of physical and/or biological attributes of the prenatal or neonatal-derived brain organoid is transferred from the sensor coupled to a recorder to the MCU.

In some embodiments, the MCU is connected to a wireless radio transmitter (RF) or a micro transmitter (MT) connecting it to at least one remote MCU.

In some embodiments, the MCU is connected to the processor/computer

In some embodiments, the system further comprises a source of stimuli/manipulation capable of delivering treatment to the prenatal or neonatal-derived brain organoid.

In some embodiments, the treatment delivered by the source of stimuli/manipulation to the prenatal or neonatal-derived brain organoid is one or more of electric pulse, optic/light stimulus, heat, a chemical agent/drug, or any combination thereof.

In some embodiments, information indicative of the treatment delivered by the source of stimuli/manipulation to the prenatal brain organoid in culture comprises information about concentration, temperature, duration, intensity, frequency and/or amplitude of the stimuli, or any combination thereof.

In some embodiments, the at least one signal and/or attribute detected and archived is optic/light signal or electric signal; In some embodiments, the optic/light signal or electric signal comprises information indicative of physical and/or biological attributes of the prenatal or neonatal-derived brain organoid.

In some embodiments, the sensor coupled to a recorder capable of detecting and archiving the one or more signals and/or attributes comprises a multi-electrode array (MEA) coupled to one or more recording head stage (RHS);

In some embodiments, the at least one signal and/or attribute detected and archived by the multi-electrode array (MEA) is an electric.

In some embodiments, the electric signal or optic/light signal comprises information indicative of spontaneous or induced neuronal function/activity of the prenatal-derived brain organoid.

In some embodiments, the optic/light signal comprises information indicative of neuronal function/activity is emitted from a genetic reporter.

In some embodiments, the information indicative of the neuronal function/activity comprises duration, intensity, frequency and/or amplitude of the detected signal.

In some embodiments, the information indicative of the neuronal function/activity further comprises spatial information.

In some embodiments, the information indicative of the neuronal function/activity is transferred from the sensor coupled to a recorder to one or more of same or different MCU.

In some embodiments, the MCU is connected to the processor/computer

In some embodiments, the system is used for drug screening and/or evaluation of treatment with a drug applied directly in culture of the prenatal or neonatal-derived brain organoid and/or of the PD-derived brain organoid. Each possibility is a separate embodiment.

In some embodiments, the drug is one or more of a psychiatric/neurologic drug in medical use for treating PD, In some embodiments, the psychiatric/neurologic drug is one or more of selective serotonin reuptake inhibitors (SSRIs), selective serotonin and norepinephrine inhibitors (SNRIs), beta-blockers, stimulants, serotonergic drugs, tricyclic antidepressants, atypical antipsychotic agents, lithium, alpha-2 agonists. Each possibility is a separate embodiment.

In some embodiments, the brain organoid comprising prenatal or neonatal-derived cells for assessment of a psychiatric disorder (PD) in human prenatal and/or neonatal cells, wherein the brain organoid is generated from the human prenatal or neonatal cells.

In some embodiments, the PD comprises one or more of Autism Spectrum Disorders (ASD), Bipolar disorder, Attention Deficit Hyperactivity Disorder (ADHD/ADD), Schizophrenia, Depression, Obsessive-Compulsive Disorders (OCD), Epilepsy, or any combination thereof.

In some embodiments, the PD comprises non-genetic psychiatric disorder (non-genetic PD).

In some embodiments, the brain organoid comprising at least a cerebral and/or cortical tissue.

In some embodiments, the brain organoid comprises a change in at least one biological and/or physical attribute, wherein said attribute comprises a size of the brain organoid and/or length of neural projection thereof, in comparison to a healthy brain organoid.

In some embodiments, the prenatal or neonatal-derived brain organoid comprised of cells treated to alter gene expression, to express a genetic reporter or comprised of cells exposed to one or more stimuli/manipulation comprising electric pulse, optic/light signal, heat, a chemical agent/drug, or any combination thereof.

Certain embodiments of the present disclosure may include some, all, or none of the above advantages. One or more technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The invention will now be described in relation to certain examples and embodiments with reference to the following illustrative figures.

FIG. 1 schematically illustrates an overview of the method for assessment of PD in neonatal and prenatal derived brain organoids.

FIG. 2A shows representative bright field micrograph images presenting an overview of the main stages of generating cortical brain organoids from primary cells, and comparing the morphology and development of a representative Autistic Spectrum Disorder (ASD)-derived cortical brain organoid and a healthy brain organoid. Briefly, brain organoids were generated from primary cells that were reprogrammed into stem cells by inducing their pluripotency (iPSCs) and growing them to form 3D structures according to the protocol for the preparation of cortical organoids from hiPSCs. The primary cells were obtained from a urine excretion of an ASD patient, and a healthy subject/donor. Shown are morphological/structural differences, including size and shape differences (day 6) between the ASD-derived and the healthy brain organoids, and between neural projection thereof (day 17).

FIG. 2B: shows representative bright field micrograph images presenting a population of about 30 3D cortical brain organoids, derived from the primary epithelial cells described in FIG. 2A, visible to the naked eye growing in a petri dish (I.), and an enlarged bright field image thereof acquired with a microscope (II.).

FIGS. 2C-2D show bar graphs presenting quantification of the mean organoids' size (FIG. 2C; (diameter (μm)=A) at day 6, and the mean ratio between organoids' size and the length of its neural projections (FIG. 2D; length (μm)=B) calculated as (=(A+B)/A) (μm) at day 17, for ASD-derived and healthy brain organoids corresponding to those demonstrated in FIG. 2A. (n=10-15 representative organoids in each group, three repeats, *=p<0.05, **=p<0.01, ***=p<0.001, and ****=p<0.0001).

FIG. 2E shows representative bright field micrograph images comparing the mean organoids' size 6 days after the beginning of the protocol for preparation of cortical organoids from hiPSCs, for ASD-derived and healthy brain organoids under normal conditions, and in the presence of an inhibitor or an activator. Size differences are apparent from about day 4 and at least up to about 5 months after.

FIG. 2F; shows bar graph presenting quantification of the mean organoids' size corresponding to those demonstrated in FIG. 2E (diameter (μm)=A). (n=10-15 representative organoids in each group, *=p<0.05, **=p<0.01, ***=p<0.001, and ****=p<0.0001).

FIG. 2G shows representative bright field micrograph images comparing the mean ratio between organoids' size and the length of its neural projections 17 days after the beginning of the protocol preparation of cortical organoids from hiPSCs, for ASD-derived and healthy brain organoids under normal conditions, and in the presence of an inhibitor or an activator. Differences in the ratio between organoids' size are apparent from about day 4 and at least up to about 5 months after.

FIG. 2H shows bar graph presenting quantification of the mean ratio between organoids' size and the length of its neural projections corresponding to those demonstrated in FIG. 2G calculated as (=(A+B)/A) (μm). (n=10-15 representative organoids in each group, *=p<0.05, **=p<0.01, ***=p<0.001, and ****=p<0.0001).

FIG. 2I shows representative bright field micrograph images bar graphs presenting the mean organoids size, and quantification of the measured diameter (A) (μm), of Shank3-derived organoids (a sub-population of ASD) and healthy brain organoids, at day 10 (n=13-18 representative organoids in each group, *=p<0.05, **=p<0.01, ***=p<0.001, and ****=p<0.0001)).

FIG. 2J shows representative bright field micrograph images presenting differences in the amount/expression of neural projections (sprouting) for ASD-derived and healthy brain organoids, at day 18. ASD-derived brain organoids exhibit either under or over expression of neuronal projection compared to healthy-derived brain organoids.

FIGS. 3A-3C show two representative fluorescence microscope images of dendritic projections including dendritic Spines from 6-month-old healthy (FIG. 3A) and ASD-derived (FIG. 3B) brains organoids dissociated to 2D neuronal culture after formation of a 3D organoid. Dendritic spines of ASD-derived organoid are clearly visible, at higher density (increased number of spines) and volume (FIG. 3C). The 3D organoids were dissociated with Papain using a commercial papain dissociation system, maintained 20 days in culture and infected at day 13 with AAV1-GAC-GreenLantern virus (1/10 dilution). Confocal imaging was taken post 4% PFA fixation using the ZEISS Lsm980 microscope.

FIG. 4A: shows representative bright field micrograph images presenting ‘an overview’ of the main stages of generating cortical brain organoids from primary cells. Apparent morphological differences make it clear to distinguish between primary human amniotic epithelial cells (HAEpiCs) (I.), induced pluripotent stem cells (iPSCs) of an established line derived from the HAEpiCs by cellular reprogramming (HAEpiC-iPSC line) (II.), and 6-day old cortical organoid generated from cells of the HAEpiC-iPSC line (III.).

FIG. 4B: shows representative bright field micrograph images presenting a population of about 15 3D cortical brain organoids at day 130 generated from the HAEpiC-iPSC line, visible to the naked eye in a 6-well culture plate.

FIG. 4C shows representative fluorescence images presenting immunostainings of undifferentiated cells of the HAEpiC-iPSC line for expression pluripotency markers SOX2, NANOG, OCT3/4, as well as Hoechst nuclear staining. Scale bar: 100 μm.

FIG. 4D shows representative fluorescence images presenting immunostainings of a 42 days old HAEpiC-Cortical Organoid for expression of nuclear staining (Hoechst), neuronal marker (TUJ1), neural stem cell markers (SOX2), as well a merged image showing the overlap in their expression pattern presented at a scale of 350 μm (Top; I.), and zoomed in images of Enlarged Neural Vesicle/rosette presented at a scale of 50 μm (Bottom; II.). The enlarged image of a neural vesicle/rosette display neural stem cells (SOX2) around the ventricle and neurons (TUJ1) surrounding the neural stem cells.

FIG. 4E shows representative fluorescence images presenting immunostainings of two 130 days old HAEpiC-Cortical Organoids (Top; I. and Bottom; II.)), for expression of nuclear staining (Hoechst), neuronal marker (TUJ1), neural stem cell markers (SOX2), as well a merged image showing the overlap in their expression pattern presented at a scale of 500 μm.

FIGS. 5A-5D presents electrical activity measurements from brain organoids, utilizing genetic reporter imaging (FIGS. 5A-5B) and electrophysiological recording (FIGS. 5C-5D).

FIG. 5A show a microscope image presenting green fluorescence indicative of calcium influx, detected from the 2D neuronal culture derived from the HAEpiC-derived 3D cortical organoids after infection thereof with AAV encoding a genetic calcium indicator (GCaMP). Representative cells were marked as ROIs (yellow circle) for further quantification of the signal. Images were taken at 10 fps, 20× NA 0.9 dry objective using Nikon (Yokogawa) spinning disk confocal microscope equipped with incubator chamber.

FIG. 5B, shows examples traces from 6 arbitrary ROI marked in FIG. 5C, indicating change in fluorescence (ΔF/F) over time (normalized to 1). The changes in fluorescence intensity observed/recorded from the individual neuron is indicative of calcium oscillations in the representative cells.

FIG. 5C, illustrates detection of spontaneous electrical activity signal from 2D neuronal culture prepared from 60 days old HAEpiC-derived 3D cortical organoids. Dissociated neurons were cultured on a 64 electrode Multi Electrode Analysis (MEA) plate (I.) and 2 min of spontaneous signal/activity were recorded from a single electrode of a MEA (II.). Also shown is a 20-sec segment from the 2 min recording (expanded from white dashed box; III.), as well as an overlay of all detected spikes (IV.).

FIG. 5D, shows a picture presenting a non-limiting setup of a system for assessment of PD. The system components includes: (I) a customized organ-on-a chip device, including at least: a place holder for a sensor (multi-array electrode (MAE)) to be used as an organ-on-a chip interface for 2D/3D culturing of brain organoids, and a recorder (recording head stage (RHS)), wired to: (II) a stimuli/manipulation system capable of providing an electrophysiological stimulus to the brain organoid in culture, recording signals from the brain organoid, and transmit information/data indicative of neuronal function/activity derived from the signals directly to the computer; (III) a computer/processor capable of instructing the stimuli/manipulation system to provide stimuli sessions, and further determining organoid behavior activity/function recorded in response to the stimuli provided. The system is further illustrated in detail in FIGS. 7A-7B.

FIG. 5E shows an enlarged picture of the custom device of FIG. 5D (I). The device is an ‘organ-on-a chip’ designed to include (1) place holder for MEA to be used as an organ-on-a chip interface for culturing brain organoid (2) a printed circuit board (PCB) (3) conductive contact pin, RHS units and connectors (4) Connectors for Ref and grounding. The device was customized according to the design principles of the system for assessment of PD presented in FIGS. 7A-7B. The device is presented in detail in FIGS. 7C-7D.

FIGS. 5F-5G shows a microscope picture presenting the interface between a brain organoid and the multi electrode array (MEA) belonging to the customized device of FIGS. 5D-5E. The brain organoid is a PD-derived cortical brain organoid (day 90) generated from cells of an ASD patient's urine sample. The MEA has 59 ‘channels’ and is capable of sending electrophysiological stimuli in a spatiotemporal controlled manner by activating a sub-set of electrodes according to a desired pattern and parameters.

FIG. 5F shows an image presenting the interface between a 3D brain organoid and the MEA at its on/off states, wherein in the ‘off’ state no electrophysiological stimuli is provided to the 3D brain organoid (Right) and in the ‘on’ state 3 electrodes are activated (Left; red dots). The activation provides an electrophysiological stimulus to the PD-derived 3D brain organoid in a spatiotemporal controlled manner including specific patterns and parameters.

FIG. 5G shows an image presenting the interface between a dissociated brain organoid in 2D culture and the MEA. The PD-derived cells in the culture were dissociated from the brain organoid of FIG. 5F. After its formation (126 days) the 3D brain organoid was processed/enzymatically digested to dissociated cells, which were plated in 2D culture.

FIGS. 6A-6C shows an illustration of computational simulations of brain organoid behavior. The simulations are illustrated as computer games representing functional cognition assays being performed by the brain organoid as a ‘player’. The simulation performs assessment of PD severity and/or provides a likelihood of being classified as healthy or PD.

FIG. 6A presents a snapshot of a computer game simulation representing functional cognitive assessment of healthy and PD-like organoid, as an example for the type of game that can be used to simulate behavior associated with an open loop mode. A predetermined stimuli to the left or right side of the organoid (a golden coin) is ‘answered’ with a behavior response of the ‘player’ that moves (left or right) in a more stimuli-dependent manner as expected from a healthy organoid or in a more random manner as would be expected from a PD-derived organoid.

FIG. 6B presents a snapshot of a computer game simulation of a dot moving in 2D space representing functional cognitive assessment of repetitive behavior of healthy and ASD-derived organoid, as an example for the type of game that can be used to simulate behavior associated with an open loop mode. The movement represents an organoid behavior in response to a predetermined stimuli, whereas a dot that moves in a periodically manner through space (i.e., in the same pattern) is classified as ASD-derived, while a dot that moves more randomly through space is classified as healthy.

FIG. 6C presents a snapshot of a computer game simulation of two dots moving in 2D space representing functional cognitive assessment of social interaction of healthy and PD-derived brain organoids, as an example of the type of game that can be used to simulate learning-behavior associated with a closed loop mode. The simulation includes a random movement of two dots in 2D space, whereas when the dots come closer to each other interaction occurs and a positive feedback treatment is determined/executed, but when the dots move apart from each other and there is no interaction a negative feedback treatment is determined/executed.

FIG. 6D shows an illustration exemplifying the process determining of a brain-organoids behavior, based on electrical activity data recorded in response to one or more treatment/stimuli session(s). Once electrophysiological raw data (or reporter activity data) is recorded in response to a stimuli session, a response pattern and pattern analysis are performed to learn about the neural network response. In each cycle, stimuli are provided through specific electrodes and the collected activity data from each electrode is filtered and reduced to record only the time in which spikes were detected (Left). Then, in following sessions, multiple repetitions of this stimulation-recording cycle are performed. The mean response pattern is computed taking into account the spike channel (which electrodes detected the signal, i.e., distribution) and the time since the stimulation was plotted (i.e., time after stimuli) (Right). This response pattern is then taken to pattern analysis that compares it to patterns/predicted patterns from healthy and PD samples. Hence determining a behavior includes at least a spatiotemporal analysis of spike propagation.

FIGS. 7A-7B present the system for assessment of a psychiatric disorder (PD).

FIG. 7A schematic non-limiting illustration of system components (dashed boxes), their structural and functional relations and workflow of the process of determining severity of PD and classifying a brain organoid based on the organoid response to a predetermined treatment (Open loop), or to positive or negative feedback treatment (Closed loop). The system includes: (1) a brain organoid in 2D/3D culture; (2) a sensor (multi-array electrode (MAE)) coupled to a recorder (recording head stage (RHS)) capable of detecting and recording signals from the brain organoid; (3) a micro-controller unit (MCU) configured to receive, integrate and/or transmit information/data indicative of neuronal function/activity derived from the signals; (4) a computer/processor capable of determine response to stimuli/treatment sessions, and give instructions to provide a predetermined treatment (Open loop), or a feedback treatment (Closed loop), and computing an output at the end of the sessions; (5) Optionally, a visualization component such as a screen/monitor/robot, or the like, connected to the processor/computer; and capable of presenting the computational simulation (i.e., the stimuli-response sessions/simulation) in a visual manner (6) a stimuli/manipulation system connected to the processor/computer and capable of delivering treatment to the brain organoid in culture. (7) The system computes an output including computation of the overall responses (Open loop), or a learning responses (Closed loop), for PD, healthy or undetermined brain organoids derived from any developmental stage (e.g., prenatal, mature, mature baby, or adult); and (8) assesses probability of severity of PD based on similarity between the organoids response and classify accordingly (9) thereby providing a platform that can be utilized for drug screening and/or as a mean for personalized medicine aiming at evaluation/prediction of clinical success of treatment with a psychiatric drug, including a medicament for neurologic, neurodevelopmental, and neurodegenerative disease.

FIG. 7B schematic illustration of the same system components of FIG. 7A, and relation between them, in basic and scalable formats. The upscaled structure includes for some of the components a plurality of units, including a plurality of cultures brain organoids, plurality of RHS, plurality of MEA, and plurality of monitors.

FIGS. 7C-7D shows a picture presenting a device customized according to the design principles of the system for assessment of PD presented in FIGS. 7A-7B. The device is an ‘organ-on-a chip’ designed to facilitate electrophysiological recordings from a biological sample. Shown are MEA positioning area, electrode connector pins, reference and ground pins, amplifying head stage, and 16 pin omnetics connector (FIG. 1C). Also shown are gold-plated pogo pins, removable connection bridges, amplifying head stage, and 16 pin omnetics connector, a grounding cable, and a copper foil cover for sealing of a plate with a 3D brain organoid or 2D neuronal culture thereof (FIG. 1D).

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

According to an aspect of the disclosure, there is provided a method for prenatal and/or neonatal assessment of psychiatric disorder (PD), the method comprising (i) obtaining human prenatal or neonatal cells; (ii) generating a prenatal or neonatal derived-brain organoid from the human prenatal or neonatal cells; and (iii) assessing one or more signal(s) and/or attribute(s) of the brain organoid.

Advantageously, the herein-disclosed method provides a prenatal or neonatal assessment of PD based on an assessment of a change/difference in one or more signal(s) and/or attribute(s) of the brain organoid, tissue and/or cells thereof.

As used herein, the singular forms “a”, “an”, and “the” are intended to include the plural forms as well, unless the context clearly indicates otherwise. For example, referring to a psychiatric disorder (PD) may include more than a single PD, and when a reference is made to the brain organoid it may include a reference to multiple brain organoids.

As used herein, the term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

As used herein, the term “comprising” is synonymous with the terms “including,” “containing,” or “characterized by,” and is inclusive or open-ended i.e. does not exclude additional, unrecited elements. According to some embodiments, the term comprising may be replaced with the term “consisting of” which excludes any element, step, or ingredient not specified.

As used herein, the term “plurality” may refer to brain organoids, and include according to some embodiments, a quantity of more than 5, more than 25, more than 50, more than 100, more than 250, more than 500, or more than 1000 brains. Each possibility is a separate embodiment.

As used herein, the term “prenatal and/or neonatal assessment” indicates that the assessment relies on the period and stage of embryonic development, fetal development, or the neonatal period and stage, and further indicates that the assessment utilizes cells derived from an embryo, a fetus, or a neonate, and obtained during the corresponding period and stage.

As used herein, the term “prenatal” refers to the period and stages of human prenatal development that starts with fertilization and ends with birth. Prenatal development begins with embryonic development and continues in fetal development until birth. In accordance, the term prenatal may refer to an embryo or a fetus. In accordance, in some embodiments, “prenatal cells” are derived from an embryo or a fetus.

The term “embryo” refers to the initial stage of human development that begins just after fertilization of the female egg cell by the male sperm (i.e., gametes). An embryo may result from sexual intercourse, intrauterine insemination (IUI), or in vitro fertilization (IVF), including any process/type of assisted reproductive technology (ART) involved in fertility treatment, including but not limited to, for example, fertility medication, embryo transfer, intracytoplasmic sperm injection (ICSI), cryopreservation, assisted zona hatching (AZH), transvaginal ovum retrieval (OVR), and others.

The term “fetus” refers to the stage of human development that begins from about the ninth week after fertilization and continues until birth.

As used herein, the term “neonatal” refers to the period and stages that follow human pregnancy from the moment of birth of a newborn/neonate and up to about 2 months. A neonate includes a premature newborn/neonate (i.e., preterm born alive before 37 weeks of pregnancy are completed), and during the first 60 days after his birth.

As used herein, the term “early neonatal stage” refers to the period during the first 7 days after birth.

In some embodiments, the brain organoid is derived from cells obtained from a neonate between 0 and about 60 days old, 0 and about 45 days old, 0 and about 30 days old, or between 0 and 7 days old. Each possibility is a separate embodiment.

In some embodiments, human neonatal cells are obtained from a newborn, including a premature newborn, up to 60 days from birth, up to 45 days from birth, up to 30 days from birth, or up to 7 days from birth. Each possibility is a separate embodiment.

In accordance, “neonatal cells” are cells derived from a neonate (i.e., up to about 30 days after birth).

The terms, “neonate” and “newborn” may be interchangeably used.

According to some embodiments, the human prenatal cells are obtained from an embryo. According to some embodiments, the human prenatal cells are obtained from a fetus. According to some embodiments, the human prenatal cells are obtained from a placenta.

According to some embodiments, the human prenatal cells are obtained by Chorionic Villus Sampling (CVS). According to some embodiments, the human prenatal cells are obtained by amniotic fluid test (Amniocentesis). According to some embodiments, the human prenatal cells are obtained by in-vitro fertilization (IVF). According to some embodiments, the human prenatal cells are obtained by post-mortem autopsy of an embryo or a fetus.

According to some embodiments, the human neonatal cells are obtained from a newborn. According to some embodiments, the human neonatal cells are obtained from a newborn at its early neonatal stage. According to some embodiments, the human neonatal cells are obtained from a placenta.

According to some embodiments, the human neonatal cells are obtained by a biopsy (e.g. puncture, scraping, swiping) of various tissues. According to some embodiments, the human neonatal cells are obtained from cord blood collection or blood withdrawal. According to some embodiments, the human neonatal cells are obtained from excretions or collected body fluids, such as urine, stool, sputum, vomitus, or saliva, or obtained from hair samples. Each possibility is a separate embodiment.

According to some embodiments, the human prenatal or neonatal cells are obtained from Chorionic Villus Sampling (CVS), amniotic fluid test (Amniocentesis), in-vitro fertilization (IVF), post-mortem autopsy, or biopsies of tissue stem cells such as, but not limited to, embryonic stem cells (hESCs), mesenchymal stem cells, nucleated blood stem cells, fibroblasts, epithelial cells, or keratinocytes. Each possibility is a separate embodiment.

As used herein, the term “PD cells” refers to cells obtained from a subject suffering from one or more psychiatric disorder (PD).

In some embodiments, the PD-cells comprise cells derived from an ASD patient.

In some embodiments, primary cells for generation of a brain organoid include, for example, but are not limited to, epithelial cells, fibroblasts, tissue-specific stem cells, nucleated blood cells, embryonic stem cells (hESCs), mesenchymal stem cells or hair keratinocytes. Each possibility is a separate embodiment.

In some embodiments, primary cells may be obtained from, for example, but not limited to, excretions or collected body fluids, such as urine, stool, sputum, vomitus or saliva, or obtained from hair samples. Each possibility is a separate embodiment.

In some embodiments, primary cells may be obtained from, for example, but not limited to, a blood withdrawal/blood test, or a biopsy (e.g. puncture, scraping, swiping) of various tissues. Each possibility is a separate embodiment.

According to some embodiments, PD-derived organoid is generated from PD cells. According to some embodiments, healthy organoid is generated from healthy cells.

According to some embodiments, PD cells or healthy cells may include for example, but are not limited to epithelial cells, fibroblasts, tissue-specific stem cells, nucleated blood cells, embryonic stem cells (hESCs), mesenchymal stem cells, or hair keratinocytes. Each possibility is a separate embodiment.

According to some embodiments, PD cells or healthy cells may be obtained from for example, but not limited to excretions or collected body fluids, such as urine, stool, sputum, vomitus, or saliva, or obtained from hair samples, a blood withdrawal/blood test, or a biopsy of a subject suffering from a psychiatric disorder (PD). Each possibility is a separate embodiment.

According to some embodiments, the PD cells or healthy may be obtained from a baby (i.e., about 2 month-2 years). Each possibility is a separate embodiment. According to some embodiments, the PD cells or the healthy cells are obtained from a toddler (i.e., about 2-4 years). Each possibility is a separate embodiment. According to some embodiments, the PD cells or healthy are obtained from a child (i.e., about 5-12 years). Each possibility is a separate embodiment. According to some embodiments, the PD cells or the healthy cells are obtained from a teen (i.e., about 13-19 years). Each possibility is a separate embodiment. According to some embodiments, the PD cells or healthy are obtained from an adult (i.e., older than >about 20 years). Each possibility is a separate embodiment.

As used herein, the term “adult” may collectively refer to an adult-PD including a toddler, a child, a teen, or an adult. According to some embodiments, adult includes a toddler, a child, a teen, or an adult.

As used herein, the term “healthy” may refer to a subject, a brain organoid and/or a cell, and is used to distinguish between a healthy state and a state of having at least one psychiatric disorder (PD), as well as to distinguish between a healthy state and a state of uncertainty as to whether a psychiatric disorder PD exists or not.

As used herein, the term “subject”, “patient” or “individual” may be used interchangeably and generally refer to a human, at any stage of human development including an embryo, a fetus, a neonate, a baby, a toddler, a child, a teen, or an adult.

The subject assessed/diagnosed for PD by the method of the present invention is preferably an embryo, a fetus, or a neonate up to 60 days old.

A subject may be a healthy subject, a subject suffering from a psychiatric disorder (PD) (i.e., a suffering subject), or a subject with respect to whom it is unknown whether he is a healthy subject or a suffering subject (i.e., subject that suffers from PD). For example, a subject may be an in vitro embryo or fetus that is a result of IVF.

The term “genetic psychiatric disorder” (“genetic PD”) refers to a psychiatric disorder in which a single mutation or a collection of mutations is known to lead to the development of pathology at a high probability (i.e., high-risk genetic markers are involved) with very minor or even completely without an involvement of environmental risk factors.

As used herein genetic psychiatric disorder (genetic PD) is distinguished from non-genetic psychiatric disorder (non-genetic PD).

As used herein, the term “non-genetic psychiatric disorder” (“non-genetic PD”) refers to a psychiatric disorder in which non-genetic factors fundamentally influence the risk and contribute to the etiology of the disorder along with low-risk genetic markers, therefore, especially in these disorders, a complex and multifactorial contributions of genes and environment co-exist, assumingly as early as prenatal development begins.

Therefore, the disclosed prenatal or neonatal assessment of the present invention is especially advantageous for non-genetic PD, as it discloses one or more signal(s) and/or attributes(s) that characterize PD-derived organoids, making them distinguishable from healthy organoids, just by visualizing structural phenotypes related to the organoid and its projections.

According to some embodiments, the psychiatric disorder (PD) comprises genetic psychiatric disorder (genetic PD). According to some embodiments, the psychiatric disorder (PD) comprises non-genetic psychiatric disorder (non-genetic PD). According to some embodiments, the psychiatric disorder (PD) comprises at least one genetic psychiatric disorder (genetic PD) and/or at least one non-genetic psychiatric disorder (non-genetic PD). Each possibility is a separate embodiment.

PD in relation to the herein-disclosed invention preferably include complications of cognitive impairment (e.g., adaptive learning), social problems (e.g., social communication, social interaction) and/or repetitive and restricted patterns of behavior. Without being bound to the theory some non-limiting examples of PD include, but are not limited to, Autism Spectrum Disorders (ASD), Bipolar disorder, Attention Deficit Hyperactivity Disorder (ADHD/ADD), Schizophrenia, Depression, Obsessive-Compulsive Disorders (OCD) and Epilepsy.

According to some embodiments, the PD includes one or more of Autism Spectrum Disorders (ASD), Bipolar disorder, Attention Deficit Hyperactivity Disorder (ADHD/ADD), Schizophrenia, Depression, Obsessive-Compulsive Disorders (OCD), and Epilepsy, or any combination thereof. Each possibility is a different embodiment.

According to some embodiments, the PD is selected from one or more of Autism Spectrum Disorders (ASD), Bipolar disorder, Attention Deficit Hyperactivity Disorder (ADHD/ADD), Schizophrenia, Depression, Obsessive-Compulsive Disorders (OCD), and Epilepsy, or any combination thereof. Each possibility is a different embodiment.

According to some embodiments, the PD comprises Autism Spectrum Disorders (ASD).

According to some embodiments, the PD comprises complications of cognitive impairment (e.g., adaptive learning). According to some embodiments, the PD comprises complications of social problems (e.g., social communication, social interaction); According to some embodiments, the PD comprises complications of repetitive behavior.

As used herein, the term “psychiatric disorder (PD)” refers to a range of disorders that affect mental, emotional and/or behavioral aspects of a subject and may have a neurodevelopmental, neurodegenerative or neurological bases, in particular neurological and neurodevelopmental base, and encompass conditions associated with reduced cognitive function (i.e., cognitive functions associated with psychiatric disorder (PD)), characterized by, cognitive impairment/rigidity (e.g., adaptive learning, attention, memory), executive function (e.g. problem-solving, decision making, planning and organization), motivational aspects, social problems (e.g., social communication, social interaction) and/or repetitive and restricted patterns of behavior.

As used herein, the term “psychiatric disorder (PD)” may refer to the term “cognitive function associated psychiatric disorder (PD)”

In accordance, according to some embodiments, assessment of PD using the systems and methods of the invention encompass assessment of cognitive functions associated with psychiatric disorder (PD).

In some embodiments, the systems and methods provided herein include assessment of cognitive functions associated with psychiatric disorder (PD).

In some embodiments, the PD encompasses PD. In some embodiments, the PD encompasses cognitive functions associated with PD.

In some embodiments, the PD includes cognitive functions associated with PD.

In some embodiments, the PD includes conditions having neurodevelopmental, neurodegenerative, and/or neurological bases. each possibility is a separate embodiment.

In some embodiments, the neurodevelopmental, neurodegenerative, and/or neurological conditions include cognitive functions associated with psychiatric disorder (PD). Each possibility is a separate embodiment.

In some embodiments, the PD includes conditions having mental, emotional and/or behavioral aspects. each possibility is a separate embodiment.

In some embodiments, conditions having mental, emotional and/or behavioral aspects include conditions having cognitive functions associated with PD includes. each possibility is a separate embodiment.

In some embodiments, the PD or the cognitive functions associated with PD includes cognitive impairment/rigidity (e.g., adaptive learning, attention, memory), executive function (e.g. problem-solving, decision making, planning and organization), motivational aspects, social problems (e.g., social communication, social interaction) and/or repetitive and restricted patterns of behavior, or any combination thereof. Each possibility is a separate embodiment.

Without being bound to the theory non-limiting examples of PD include, Autism Spectrum Disorders (ASD), Bipolar disorder, Attention Deficit Hyperactivity Disorder (ADHD/ADD), Schizophrenia, Major Depression, Obsessive-Compulsive Disorders (OCD), and Epilepsy.

According to some embodiments, PD or condition having cognitive functions associated with PD comprises one or more of Autism Spectrum Disorders (ASD), Bipolar disorder, Attention Deficit Hyperactivity Disorder (ADHD/ADD), Schizophrenia, Major Depression, Obsessive-Compulsive Disorders (OCD), Rett syndrome, Fragile X Syndrome, Intellectual Developmental Disorder, Down Syndrome, Williams Syndrome, Prader-Willi Syndrome, Angelman Syndrome, Smith-Magenis Syndrome, Epilepsy, Parkinson's disease, and Alzheimer's disease, or any combination thereof. Each possibility is a different embodiment.

According to some embodiments, the PD or conditions having cognitive functions associated with PD are selected from one or more of Autism Spectrum Disorders (ASD), Bipolar disorder, Attention Deficit Hyperactivity Disorder (ADHD/ADD), Schizophrenia, Major Depression, Obsessive-Compulsive Disorders (OCD), Rett syndrome, Fragile X Syndrome, Intellectual Developmental Disorder, Down Syndrome, Williams Syndrome, Prader-Willi Syndrome, Angelman Syndrome, Smith-Magenis Syndrome, Epilepsy, Parkinson's disease, and Alzheimer's disease, or any combination thereof. Each possibility is a different embodiment.

According to some embodiments, PD comprises Autism Spectrum Disorders (ASD).

As used herein, the term “Autism Spectrum Disorders” (“ASD”) refers to a range of neurodevelopmental disorders that mainly affect social and communication skills, but also include other symptoms related, for example, to learning and to repetitive behavior, and may further be associated with co-morbidities. The spectrum refers to the range of appearance of the disorder that can manifest very differently from person to person.

As used herein, the term “non-syndromic idiopathic ASD” (also known as idiopathic autism) refers to ASD in which the cause is unknown, and risk involves contributions of co-existing genetic and environmental factors, i.e., multifactorial and complex non-genetic PD.

According to some embodiments, the Autism Spectrum Disorders (ASD) comprises non-syndromic idiopathic ASD. According to some embodiments, non-genetic PD comprises non-syndromic idiopathic ASD.

As used herein, the term “organoid” refers to an in vitro, human pluripotent stem cells (hiPSC)-derived, grown, and to some level self-organized 3D tissue that resembles, at least in part of its structural, cell type composition, and functional qualities, an in vivo organ. An organoid of the present invention may include a population of cells forming a brain organoid or a brain spheroid. The terms “brain organoid” and “brain spheroid” may be interchangeably used.

According to some embodiments, a brain organoid comprises 3D organoid and/or 2D cell culture derived therefrom. Each possibility is a separate embodiment.

According to some embodiments, a brain organoid comprises 3D organoid and/or 2D tissue derived therefrom. Each possibility is a separate embodiment.

According to some embodiments, a brain organoid comprises 3D organoid and/or 3D clamps, or spheroids derived therefrom. Each possibility is a separate embodiment.

According to some embodiments, the brain organoid comprises the tissue and/or cells thereof.

As used herein, the term “brain organoid” refers to a self-organized 3D at least partially structured tissue that is derived and generated from primary cells that are reprogramed and transformed into induced pluripotent stem cells (iPSC), and then differentiated into neural progenitor cells (NPC), and further differentiated into neurons. The brain organoids of the invention include cerebral or cortical organoids. The brain organoids of the invention include organoids derived from primary cells obtained from human individuals at specific developmental stages, i.e., an embryo, a fetus, or a newborn/neonate.

The generation of a “brain organoid” relies on hiPSC ability to aggregate into embryonic bodies (EBs) and further self-organize into 3D structures that upon differentiation may contain multiple areas recapitulating/modulating an individual and specific region of the human brain or multiple different regions of the human brain, including but not limited to, for example, the cerebral region, the cortex, the forebrain, the midbrain, the retina, the hippocampus, the hypothalamus, the cerebellum, other brain regions or a mixture of these regions. A brain organoid includes a great diversity of differentiated cell types, including but not limited to, for example, neural progenitor cells (NPC), neurons, astrocytes, oligodendrocytes, and more, the differentiation of which can be unguided or unguided.

For example, “unguided differentiation” may result in a “cerebral brain organoid” that includes multiple areas of self-organize 3D structures that recapitulate and modulate the whole human brain including the cerebral region of the human brain and may include additional areas recapitulating other human brain regions, including a cortical area, the forebrain, and others. Alternatively, using a “guided differentiation” procedure, several types of “brain region-specific organoids” can be generated to recapitulate an individual region of interest of the human brain. A brain region-specific organoid includes uniform and reproducible tissue, for example, a “cortical neuroepithelium organoid” recapitulates only the cerebral cortex region of the human brain, a “forebrain organoid” and a “cortical spheroid organoid” also modulates only the cerebral cortex region of the human brain, a “midbrain organoid” modulates only the midbrain region of the human brain, and the like.

According to some embodiments, the brain organoid comprises multiple areas of self-organize 3D structures that recapitulate and modulate the whole human brain (i.e., cerebral brain organoid). According to some embodiments, the brain organoid comprises at least a cerebral-cortical area (i.e., cerebral brain organoid or brain region-specific organoids such as cortical neuroepithelium organoid or cortical spheroid organoid). According to some embodiments, the brain organoid includes at least a cerebral area and a cortical area (i.e., cerebral brain organoid or brain region-specific organoids). According to some embodiments, the brain organoid includes a cortical area only (“cortical brain organoid”). According to some embodiments, the brain organoid comprises at least a striatum area, at least an hippocampal area, at least a midbrain area, at least a cerebellum area, at least a spinal cord area, at least a hypo-thalamus area, at least a thalamus area, at least a basal ganglia area, at least a forebrain area, at least a midbrain area. According to some embodiments, the brain organoid comprises EB-like aggregates, cortical spheroids, cortical neuroepithelium or oligocortical spheroids. Each possibility is a separate embodiment.

In some embodiments, the prenatal and/or neonatal brain organoid includes one or more of midbrain organoid, hippocampal organoid, striatal organoid, neocortical organoid, cerebral organoid and/or cortical organoid or any combination thereof. Midbrain, hippocampal, striatal, neocortical. Each possibility is a separate embodiment.

According to some embodiments, the brain organoid comprises EB-like aggregates and/or spheroids. Each possibility is a separate embodiment.

According to some embodiments, the brain organoid comprises at least a striatum area, at least a hippocampal area, at least a midbrain area, at least a cerebellum area, at least a spinal cord area, at least a hypo-thalamus area, at least a thalamus area, at least a basal ganglia area, at least a forebrain area, at least a midbrain area, or any combination thereof. Each possibility is a separate embodiment.

According to some embodiments, brain organoids are generated using guided or unguided differentiation. Each possibility is a separate embodiment.

The term “tissue and/or cells thereof” is related to the brain organoid and may refer to the tissue and/or cells used to generate the brain organoid in 2D culture or 3D culture, prior to formation of the 3D brain organoid or to it may refer to the tissue and/or cells used to generate the brain organoid in 2D culture or 3D culture, after formation of the 3D brain organoid comprising the tissue and/or cells thereof. According to some embodiments, the tissue and/or cells thereof comprise population of cells prior to or after brain organoid formation; according to some embodiments, the tissue and/or cells thereof comprise 2D culture; according to some embodiments, the tissue and/or cells thereof comprise 3D culture and/or brain organoid. Non-limiting example of a 3D tissue/3D culture include Embryonic bodies, aggregates, brain organoids). Each possibility is a separate embodiment.

According to some embodiments, the brain organoid comprises 2D tissue and cells grown in culture; according to some embodiments, the brain organoid comprises a self-organized 3D structure grown in culture; according to some embodiments, the brain organoid comprises at least a cerebral and/or cortical tissue/area/region; according to some embodiments, the cerebral and/or cortical tissue comprises a defining 2D/3D structure, shape, and size and/or cell type composition, or combination thereof.

According to some embodiments, the method and system herein disclosed comprises 3D and/or 2D cultures comprising a brain organoid, and/or tissue and/or cells thereof.

As used herein, the term “obtaining” refers to the cells collected from a subject, in purpose of using them, in the following step, to generate a brain organoid. The cells obtained (hereinafter “the sample”) may be prenatal or neonatal, PD, or healthy cells derived and collected from the subject.

The cells obtained may be accepted, received, acquired, purchased from a third party and/or collected from a subject.

In some embodiments, the step of obtaining the cells is performed by a different party than the party that generates the brain organoid (i.e., a third party); in some embodiments, the step of obtaining the cells may be performed by a third party that collects the sample from the subject and may store it or transfer it for storage with yet another different third party, until further use is performed with the sample.

In some embodiments, the step of generating the brain organoid may be performed by a different party than the party who obtained the sample and/or transferred it to storage; in some embodiments, the step of generating the brain organoid may be performed by the same party who obtained the sample and/or transferred it to storage.

As used herein, the term “generating” refers to the in vitro procedure of producing in-culture a brain organoid comprising tissue and cells derived from prenatal, neonatal, PD, or healthy subjects. The procedure involves cell growth in culture and/or in a bioreactor, the transformation of cells, induction of pluripotency, cell expansion, cell aggregation, embryonic bodies formation, and differentiation induced using neural differentiation specific media, growth factors and/or inhibitors and/or activators and/or other physical and/or chemical cues.

According to some embodiments, generating a brain organoid comprises transforming the obtained human prenatal, neonatal, PD, or healthy cells to pluripotent stem cells (hiPSC). Each possibility is a different embodiment.

According to some embodiments, generating a brain organoid comprises transforming the obtained human prenatal, neonatal, PD, or healthy cells to hiPSC-derived Neural Progenitor Cells (NPC). Each possibility is a different embodiment.

According to some embodiments, NPC are further differentiated.

Advantageously, the herein-disclosed method provides an assessment of PD based on a change/difference in one or more signal(s) and/or attribute(s) of the brain organoid, and/or tissue and/or cells thereof.

As used herein, the term “assessment of psychiatric disorder (PD)” refers to an assessment of a change/difference in one or more signal(s) and/or attribute(s) of the brain organoid, and/or tissue and/or cells thereof. The assessment includes, primarily, visual examination of the organoid for attributes/phenotypes (e.g., external/extracellular or internal/intracellular phenotypes related to structure), but may also include examination of signals related to electrical activity/function).

In some embodiments, the attribute includes visual attribute. In some embodiments, the visual attribute includes a structural attribute.

In some embodiments, the visual attribute includes the organoid structure, including the structure of its neural projections.

As used herein, the term “visual”, “visual external” or “visual phenotype” or “visual attribute” may be used interchangeably and refer to 3D structures that can be visualized using an imaging device.

In some embodiments, the attribute includes a visual 3D structure. In some embodiments, the visual attribute includes 3D structure. In some embodiments, the visual attribute includes 3D structure.

In some embodiments, the visual 3D structure includes one or more of organoid shape, organoid size, dendritic projection shape including spines density, or any combination thereof. Each possibility is a separate embodiment.

In some embodiments, the 3D structure includes one or more of organoid shape, organoid size, dendritic projection shape, spines density, or any combination thereof. Each possibility is a separate embodiment.

In some embodiments, the 3D structure includes for example, but are not limited to, one or more of organoid shape, organoid size, dendritic projection shape, spines density, or any combination thereof. Each possibility is a separate embodiment.

In some embodiments, the 3D structure includes one or more of organoid shape, organoid size, spines density, or any combination thereof. Each possibility is a separate embodiment.

In some embodiments, the 3D structure includes external/extracellular structures or internal/intercellular structures, or any combination thereof. Each possibility is a separate embodiment.

In some embodiments, the external/extracellular structures include organoid shape/projection sprouting, organoid size, dendritic projection shape, and spines density, or any combination thereof. Each possibility is a separate embodiment.

The assessment of PD may take into consideration electrical signals indicative of behavior-like responses, including learning-behavior responses of the neural network of prenatal or neonatal derived-brain organoid or 2D cultures derived therefrom.

In some embodiments, the signal includes an electrical signal. In some embodiments, the electrical signal includes a determined brain organoid behavior.

In some embodiments, the signal includes a determined brain organoid behavior. In some embodiments, the signal includes a brain organoid behavior.

In some embodiments, the attribute includes electric signal. In some embodiments, the attribute includes a determined brain organoid behavior.

According to embodiments, the one or more signal(s) and/or attribute(s) comprise a visual phenotype/attribute of the organoid and/or an electrical activity. Each possibility is a separate embodiment.

The assessment of PD may further take into consideration and/or include computational bioinformatic data derived from gene expression or genetic/genomic examination of the prenatal or neonatal derived-brain organoid or other functional assays.

Disclosed is an advantageous and surprising biological and/or a physical attribute associated with a psychiatric disorder (PD)-derived brain organoid, wherein the biological and/or a physical attribute associated with a psychiatric disorder (PD)-derived brain organoid comprises a change/difference in one or more signal(s) and/or attribute(s) thereby advantageously providing bases for assessment of PD in a prenatal or neonatal derived-brain organoid, and/or tissue and/or cells thereof.

According to some embodiments, the assessment of PD comprises assessment of a change/difference in one or more signal(s) and/or attribute(s) of the brain organoid, and/or tissue and/or cells thereof.

According to some embodiments, a biological and/or a physical attribute is associated with a psychiatric disorder (PD)-derived brain organoid; according to some embodiments, the biological and/or a physical attribute associated with a psychiatric disorder (PD)-derived brain organoid comprises a change/difference in one or more signal(s) and/or attribute(s); according to some embodiments, the change/difference in one or more signal(s) and/or attribute(s) is evaluated in the prenatal or neonatal derived-brain organoid, and/or tissue and/or cells thereof; according to some embodiments, the change/difference in one or more signal(s) and/or attribute(s) is evaluated in brain organoid, and/or tissue and/or cells thereof derived from an embryo, fetus or neonate. Each possibility is a separate embodiment.

According to some embodiments, the assessment of PD further comprises computational bioinformatic data derived from gene expression or genetic examination of the prenatal or neonatal derived-brain organoid; according to some embodiments, the assessment of PD further comprises assessing a learning-behavior response of the prenatal or neonatal derived-brain organoid. Each possibility is a different embodiment.

As used herein, the term a “change/difference” may refer to a signal and/or attribute derived from the prenatal or neonatal-derived brain organoid in comparison to the same type of signal and/or attribute derived from a PD-derived brain organoid and/or from a healthy brain organoid. The term may also refer to a signal and/or attribute derived from the prenatal or neonatal-derived brain organoid in comparison to before and after stimuli sessions including treatment/stimuli, including electrophysiological stimuli.

Stimuli sessions may include treatment/stimuli. As used herein, the terms “treatment” or “stimuli” may refer to one or more of electrical pulse (electrophysiological stimuli), optic/light stimulus, heat, a chemical agent/drug, or any combination thereof. The treatment/stimuli may be delivered to a brain organoid in culture by the stimuli/manipulation system. The terms “treatment” or “stimuli” may be interchangeably used.

The term “parameters” may refer to concentration, temperature, duration, intensity, spatial distribution/spatiotemporal propagation, frequency and/or amplitude of the stimuli/treatment (whether predetermined or determined positive or negative feedback.

According to some embodiments, the assessment of PD comprises assessing a change/difference in one or more signal(s) and/or attribute(s) of at least a prenatal or neonatal derived-brain organoid, compared with a healthy brain organoid; according to some embodiments, the assessment of PD comprises assessing a change/difference in one or more signal(s) and/or attribute(s) of at least a prenatal or neonatal derived-brain organoid, compared with a healthy brain organoid, or to the population mean or the overall distribution of values, representing the healthy and/or pathological samples. Each possibility is a separate embodiment. According to some embodiments, the assessment of PD comprises assessing a change/difference in one or more signal(s) and/or attribute(s) of at least a prenatal or neonatal derived-brain organoid, and/or a PD-derived brain organoid, compared with a healthy brain organoid. Each possibility is a separate embodiment.

According to some embodiments, the assessment of PD comprises assessing one or more signal(s) and/or attribute(s) of the brain organoid; According to some embodiments, the assessment of PD comprises assessing one or more signal(s) and/or attribute(s) of the brain organoid cells and/or tissue thereof; according to some embodiments, the assessment of PD comprises assessing one or more signal(s) and/or attribute(s) of the cells and/or tissue forming the 3D brain organoid; according to some embodiments, the assessment of PD comprises assessing one or more signal(s) and/or attribute(s) of the cells and/or tissue in 2D culture.

As used herein, the term “signal and/or attribute” refers to any biological and/or physical/biophysical signal or attribute, functional or structural (including, for example, but not limited to external visual phenotypes/attribute o behavior-like responses of a neuronal network), indicated by a visual optic/light signal or electric signal, wherein the optic/light signal or electric signal comprises information indicative of the biophysical and/or biological attributes of the brain organoid, the cells and/or tissue thereof.

In some embodiments, the attribute includes visual attribute. In some embodiments, the visual attribute includes a structural attribute.

In some embodiments, the visual attribute includes the organoid structure, including the structure of its neural projections.

As used herein, the term “visual”, “visual phenotype” or “visual attribute” may be used interchangeably and refer to 3D structures that can be visualized using an imaging device.

In some embodiments, the visual attribute includes 3D structural elements.

In some embodiments, the attribute includes visual 3D structural elements.

In some embodiments, the 3D structural elements include one or more of organoid shape, organoid size, dendritic projection shape, spines density, or any combination thereof.

In some embodiments, the 3D structural elements include one or more of organoid shape, organoid size, spines density, or any combination thereof.

In some embodiments, the 3D structure includes one or more of organoid shape, organoid size, spines density, or any combination thereof. Each possibility is a separate embodiment.

In some embodiments, the 3D structure includes external/extracellular structures or internal/intercellular structures, or any combination thereof. Each possibility is a separate embodiment.

In some embodiments, the external/extracellular structures include organoid shape/projection sprouting, organoid size, dendritic projection shape, and spines density, or any combination thereof. Each possibility is a separate embodiment.

In some embodiments, the signal includes an electrical signal. In some embodiments, the electrical signal includes a determined brain organoid behavior.

In some embodiments, the signal includes a determined brain organoid behavior. In some embodiments, the signal includes a brain organoid behavior.

In some embodiments, the attribute includes electric signal. In some embodiments, the attribute includes a determined brain organoid behavior.

According to some aspects, the invention provides a method for prenatal and/or neonatal assessment of psychiatric disorder (PD), the method comprising:
(i) obtaining human prenatal or neonatal cells; (ii) generating a prenatal or neonatal derived-brain organoid; and (iii) assessing one or more signal and/or attribute of the brain organoid. Each possibility is a separate embodiment.

According to some embodiments, the assessing includes determining a degree of similarity between the one or more signal(s) and/or attribute(s) of the prenatal or neonatal brain organoid and PD-derived organoids, or between the one or more signal(s) and/or attribute(s) of the prenatal or neonatal brain organoid and healthy organoids. Each possibility is a separate embodiment.

In some additional embodiments, the method includes classifying the prenatal or neonatal organoid as ‘PD’ or ‘healthy’ based on the determined degree of similarity. Each possibility is a separate embodiment.

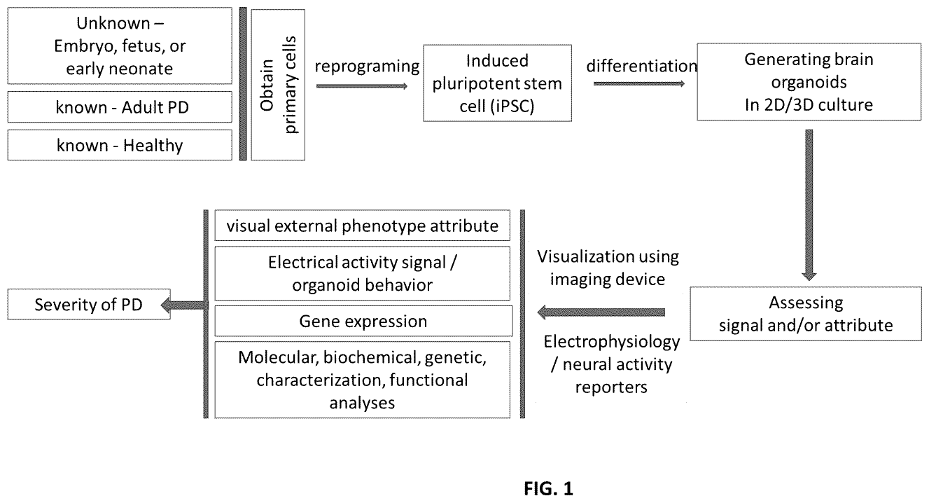

Reference is now made to FIG. 1 illustrating an overview of the method for assessment of PD in neonatal and prenatal derived brain organoids.

In some further additional embodiments, the method further includes scoring or classifying the prenatal or neonatal brain organoid according to ‘severity of PD’ based on the determined degree of similarity. Each possibility is a separate embodiment.

According to some embodiments, the PD includes non-genetic psychiatric disorder (non-genetic PD).

In some specific embodiments, the PD includes one or more disease selected from of Autism Spectrum Disorders (ASD), Bipolar disorder, Attention Deficit Hyperactivity Disorder (ADHD/ADD), Schizophrenia, Depression, Obsessive-Compulsive Disorders (OCD), and Epilepsy, or any combination thereof. Each possibility is a separate embodiment.

In some further specific embodiments, the PD includes an Autistic Spectrum Disorder (ASD).

In some further specific embodiments, the ASD includes non-syndromic idiopathic ASD.

According to some embodiments, the prenatal or neonatal cells are obtained from one or more of an embryo, a fetus, and a newborn, or any combination thereof. Each possibility is a separate embodiment.

In some embodiments, a newborn in less than 60 days old, less than 30 days old, or less than 7 days old.

According to some specific embodiments, the prenatal or neonatal cells are obtained by one or more of Chorionic Villus Sampling (CVS), amniotic fluid test (Amniocentesis), in-vitro fertilization (IVF), cord blood collection, and/or biopsies, or any combination thereof. Each possibility is a separate embodiment.

In some specific embodiments, the prenatal or neonatal cells include Human Amniotic Epithelial Cells (HAEpiC). Each possibility is a separate embodiment. According to some embodiments, generating a prenatal or neonatal derived-brain organoid includes reprogramming the obtained prenatal or neonatal cells to induced pluripotent stem cells (hiPSC). Each possibility is a separate embodiment.

In some embodiments, generating a prenatal or neonatal derived-brain organoid includes generating Neural Progenitor Cells (NPC) from the iPSCs. Each possibility is a separate embodiment.

In some embodiments, generating a prenatal and/or neonatal derived-brain organoid includes further differentiation of NPC to neurons. Each possibility is a separate embodiment.

In some embodiments, generating a prenatal or neonatal derived-brain organoid includes generating neurons directly from the iPSCs. Each possibility is a separate embodiment. (i.e., without stopping at NPC)

In some related embodiments, the prenatal and/or neonatal brain organoid includes cerebral and/or cortical tissue. Each possibility is a separate embodiment.

In some related embodiments, the prenatal and/or neonatal brain organoid includes neural vesicle/rosette structures including neural stem cells (SOX2) around ventricles, and neurons (TUJ1) surrounding the neural stem cells. Each possibility is a separate embodiment.

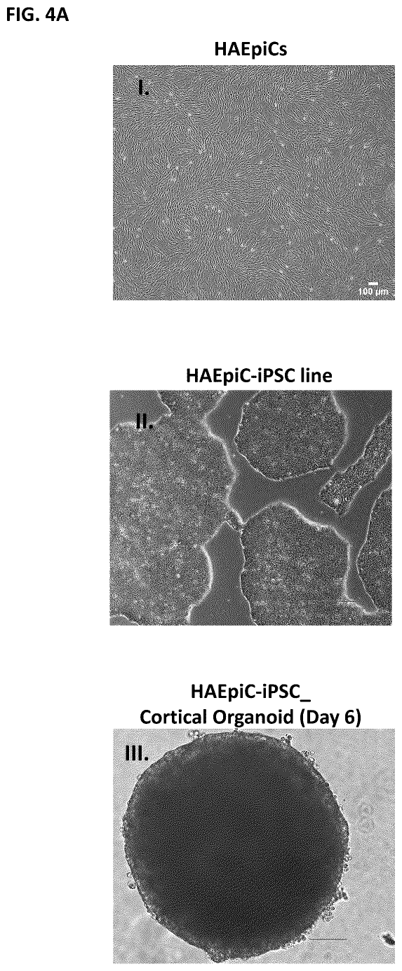

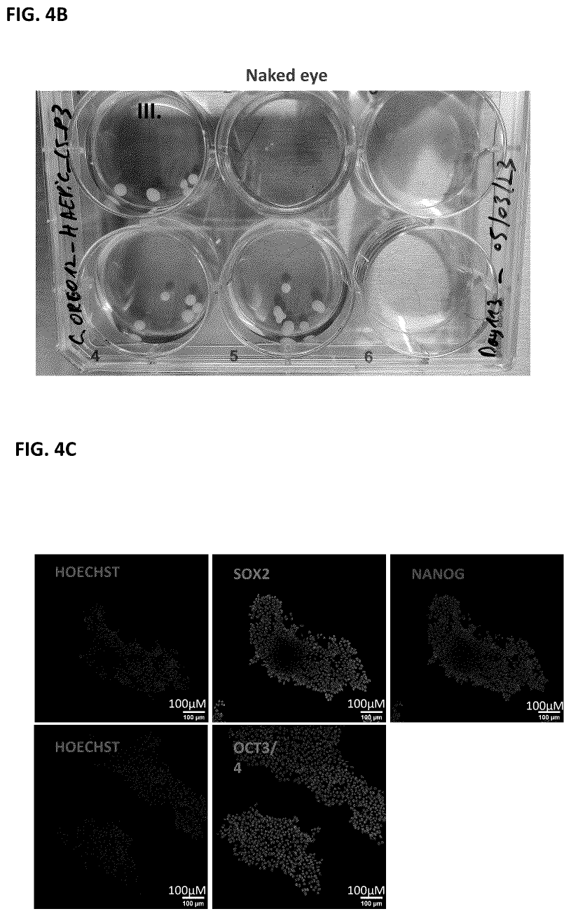

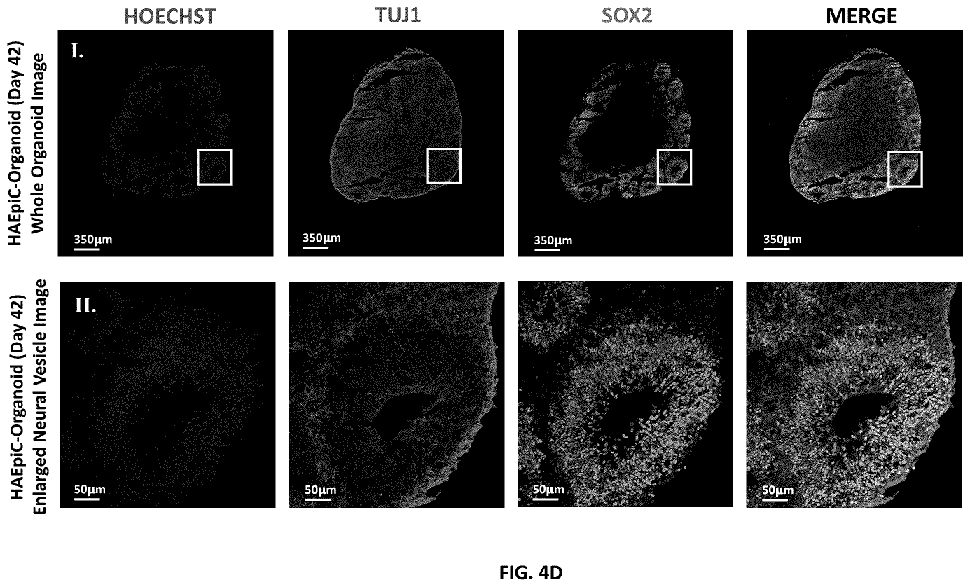

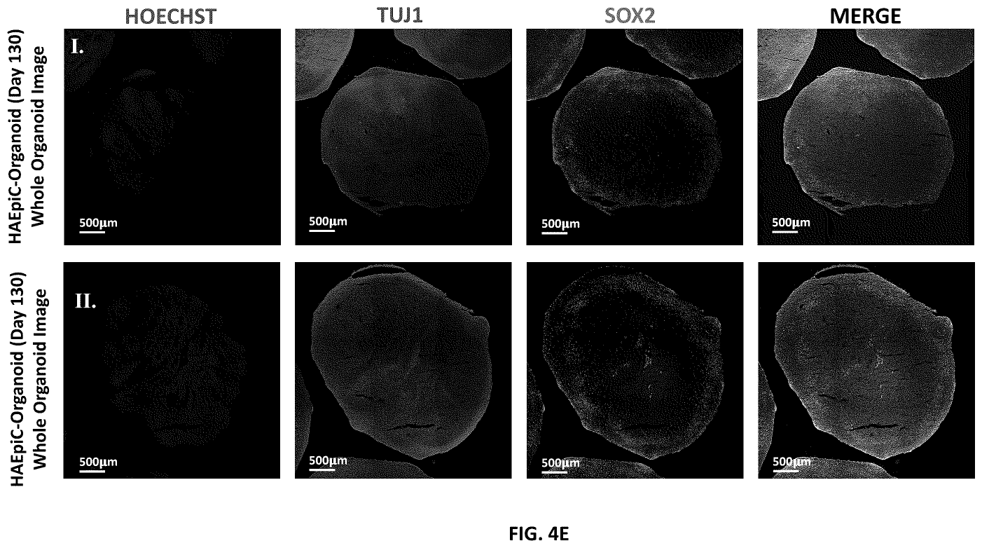

Reference is made to Example 3, FIGS. 4A-4E presenting the main stages of generating prenatal or neonatal organoids.

According to some embodiments, the one or more attributes(s) include visual external phenotypes of the organoid and/or neural projections thereof selected from one or more of: cell/tissue morphology, 3D structural elements (such as projection and spines), shape and/or size, or any combination thereof. Each possibility is a separate embodiment.

In some embodiments, visualizing external phenotypes includes imaging of the organoid and/or neural projections thereof. Each possibility is a separate embodiment.

In some embodiments, the imaging includes, for example, but is not limited to illumination and contrast methods, and fluorescence methods.

According to some embodiments, the one or more signal(s) and/or attribute(s) are selected from visual external phenotype and/or electrophysiological response/signal. Each possibility is a separate embodiment.

In some embodiments, the visual external phenotype includes the organoid and/or neural projections thereof. Each possibility is a separate embodiment.

According to some embodiments, the one or more signal(s) and/or attribute(s) are selected from one or more of: organoid shape, organoid size, dendritic projection shape, spine density, and electrophysiological response/signal, or any combination thereof. Each possibility is a separate embodiment.

According to some embodiments, the one or more attribute(s) include a visual external phenotype of the organoid and/or neural projections thereof; and wherein the visual phenotype includes one or more of: organoid shape, organoid size, dendritic projection shape, spine density, or any combination thereof. Each possibility is a separate embodiment.

According to some embodiments, the one or more attribute(s) is a visual attribute.

In some specific embodiments, the one or more attribute(s) include a deviation in the size of an ASD-derived organoid from a healthy organoid.

In some embodiments, deviation in the size of an organoid includes increment or reduction in the size. Each possibility is a separate embodiment.

In some specific embodiments, organoid size includes organoid diameter (A) (μm). In some specific embodiments, organoid size includes a ratio between the organoid diameter (A) (μm) and its outwards neural projection (B) (μm).

In some specific embodiments, the one or more attribute(s) include a deviation in the shape of ASD-derived organoid relative to healthy organoid.

In some specific embodiments, differences in the shape of an organoid include increment or reduction in the number of projection (over or under sprouting). Each possibility is a separate embodiment.

According to embodiments, the one or more attribute(s) include organoid shape and/or size. Each possibility is a separate embodiment.

In some specific embodiments, the assessing of the attribute of organoid size includes determining organoid diameter (A) (μm).

In some specific embodiments, the mean organoid diameter (A) of PD-derived organoids is at least about 25 μm greater or smaller than mean organoid diameter of healthy organoids. Each possibility is a separate embodiment.

wherein assessing the attribute of organoid shape and/or size comprises incubating the organoid with an activator or an inhibitor before determining organoid diameter (A) (μm).

In some embodiments, the assessing of the attribute of organoid size includes incubating the organoid with an activator before determining organoid diameter (A) (μm).

In some embodiments, the activator or the inhibitor are WNT signaling pathway regulators.

In some embodiments, the activator includes activator of the WNT signaling pathway. In some specific embodiments, the activator of the WNT signaling pathway is CHIR99021.

In some embodiments, the assessing of the attribute of organoid shape and/or size includes determining a ratio between the organoid diameter (A) (μm) and its outwards neural projection (B) (μm). Each possibility is a separate embodiment.

In some specific embodiments, the ratio is calculated as ((A+B)/A) (μm).

In some specific embodiments, the mean ratio between the organoid diameter (A) (μm) and its outwards neural projection (B) (μm) of PD-derived organoids is at least about 20% greater than the mean ratio of healthy organoids; and wherein the ratio is calculated as ((A+B)/A) (μm).

In some embodiments, an PD-derived brain organoid may have a size/diameter (A) at least about 20 μm greater, at least about 30 μm greater, at least about 40 μm greater, at least about 50 μm greater, at least about 60 μm greater, at least about 70 μm greater, at least about 80 μm greater, at least about 90 μm greater, at least about 100 μm greater, at least about 120 μm greater, at least about 140 μm greater, at least about 160 μm greater, at least about 180 μm greater, or at least about 200 μm greater than a size/diameter (A) of a healthy organoid. Each possibility is a separate embodiment.

In some embodiments, an PD-derived brain organoid may have a size/diameter (A) at least about 20 μm smaller, at least about 30 μm smaller, at least about 40 μm smaller, at least about 50 μm smaller, at least about 60 μm smaller, at least about 70 μm smaller, at least about 80 μm smaller, at least about 90 μm smaller, at least about 100 μm smaller, at least about 120 μm smaller, at least about 140 μm smaller, at least about 160 μm smaller, at least about 180 μm smaller, or at least about 200 μm smaller than a size/diameter (A) of a healthy organoid. Each possibility is a separate embodiment.

In some embodiments, an PD-derived brain organoid may have a size/diameter (A) that deviates from the size of a healthy organoid by at least about 20 μm, by at least by about 30 μm, by at least about 40 μm, by at least about 50 μm, by at least about 60 μm, by at least about 70 μm, by at least about 80 μm, by at least about 90 μm, by at least about 100 μm, by at least about 120 μm, by at least about 140 μm, by at least about 160 μm, by at least about 180 μm, or by at least about 200 μmeter (A). Each possibility is a separate embodiment.

In some embodiments, an PD-derived brain organoid may have a size/diameter (A) between about 20 μm and about 200 μm, between about 50 μm and about 160 μm greater than the size/diameter (A) of a healthy organoid. Each possibility is a separate embodiment.

In some embodiments, a PD-derived brain organoid may have a size/diameter (A) between about 20 μm and about 200 μm, between about 20 μm and about 160 μm smaller than the size/diameter (A) of a healthy organoid. Each possibility is a separate embodiment.

In some embodiments, the incubation of the organoids with the activator increase size/diameter (A) differences between PD-derived organoids and healthy organoids by more than about 5 μm, more than about 10 μm, more than about 15 m, more than about 20 μm, more than about 25 μm, more than about 30 μm, more than about 35 μm, more than about 40 μm, more than about 45 μm, more than about 50 μm, or more than about 55 μm, or more than about 60 μm, or more.

In some embodiments, PD-derived brain organoid has a size/diameter greater or smaller than a size/diameter (A) of a healthy from about day 4 and at least up to 5 months after; wherein day=0 is the beginning of the protocol for preparation of cortical organoids from hiPSCs.

In some embodiments, PD-derived brain organoid has a size/diameter greater or smaller than a size/diameter (A) of a healthy from about day 4 and at least up to day 6; wherein day=0 is the beginning of the protocol for preparation of cortical organoids from hiPSCs.

In some embodiments, the organoid's size/diameter is determined at about day 6 or any time from about day 6, and at least up to about 5 months; wherein day=0 is the beginning of the protocol for preparation of cortical organoids from hiPSCs. Each possibility is a separate embodiment.

In some embodiments, an PD-derived brain organoid may have a ratio between the organoid size and its outwards neural projection at least about 20% greater, at least about 25% greater, at least about 30% greater, at least about 40% greater, at least about 45% greater, at least about 50% greater, or at least about 60% greater, than a ratio between a healthy organoid size and its outwards neural projection. Each possibility is a separate embodiment. In some embodiments, the ration is calculated as ((A+B)/A).

In some embodiments, an PD-derived brain organoid may have a ratio between the organoid size and its outwards neural projection at least about 20% greater or smaller, at least about 25% greater or smaller, at least about 30% greater or smaller, at least about 40% greater or smaller, at least about 45% greater or smaller, at least about 50% greater or smaller, or at least about 60% greater or smaller, than a ratio between a healthy organoid size and its outwards neural projection. Each possibility is a separate embodiment. In some embodiments, the ration is calculated as ((A+B)/A).

In some embodiments, an PD-derived brain organoid may have a ratio between the organoid size/diameter (A) and its outwards neural projection of between about 15% and about 75%, between about 25% and about 70%, or between about 40% and about 60%, greater or smaller than a ratio between a healthy organoid size and its outwards.

In some embodiments, PD-derived brain organoid has a ratio between the organoid size/diameter (A) and its outwards neural projection greater or smaller than a ratio of a healthy organoid from about day 10 and at least up to 5 months; wherein day=0 is the beginning of the protocol for preparation of cortical organoids from hiPSCs. Each possibility is a separate embodiment.

In some embodiments, PD-derived brain organoid has a ratio between the organoid size/diameter (A) and its outwards neural projection greater or smaller than a ratio of a healthy organoid from about day 10 and at least up to day 17 day; wherein day=0 is the beginning of the protocol for preparation of cortical organoids from hiPSCs. Each possibility is a separate embodiment.

In some embodiments the ration is calculated as ((A+B)/A).

In some embodiments, the organoid's ratio between the organoid size and its outwards neural projections is determined at about day 17, or from about day 10 and at least up to about 2 months; wherein day=0 is the beginning of the protocol for preparation of cortical organoids from hiPSCs. Each possibility is a separate embodiment.

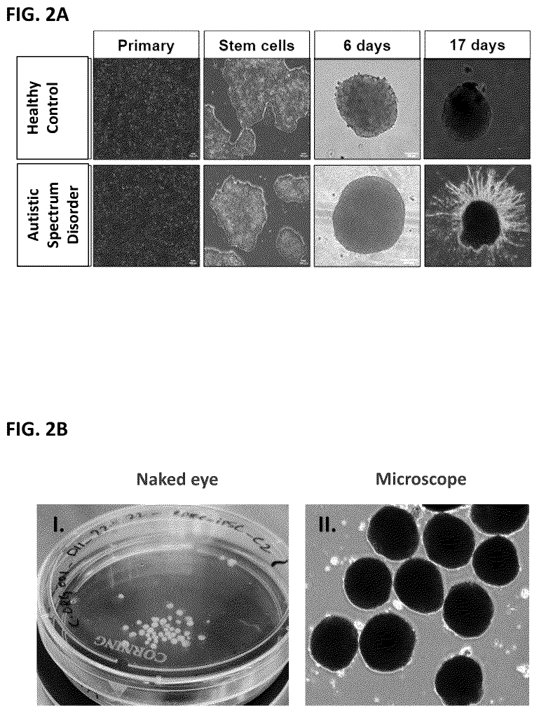

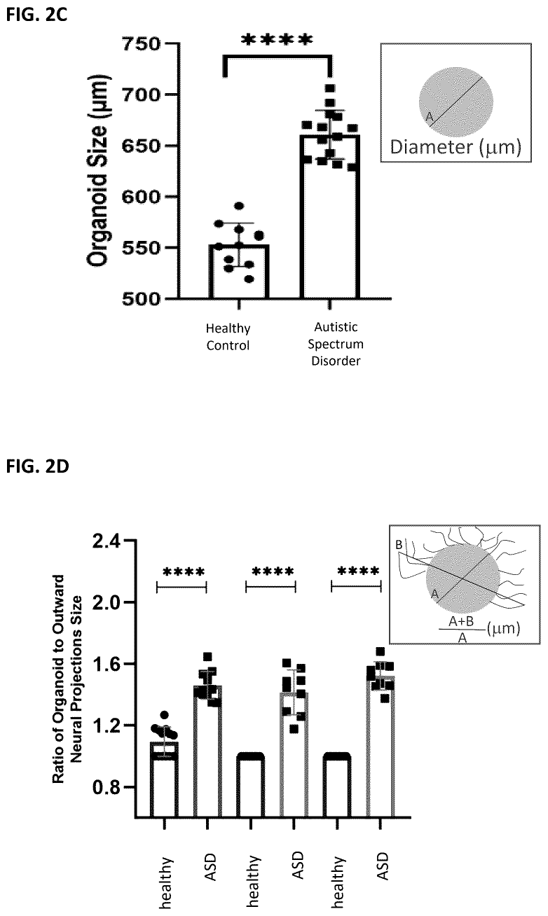

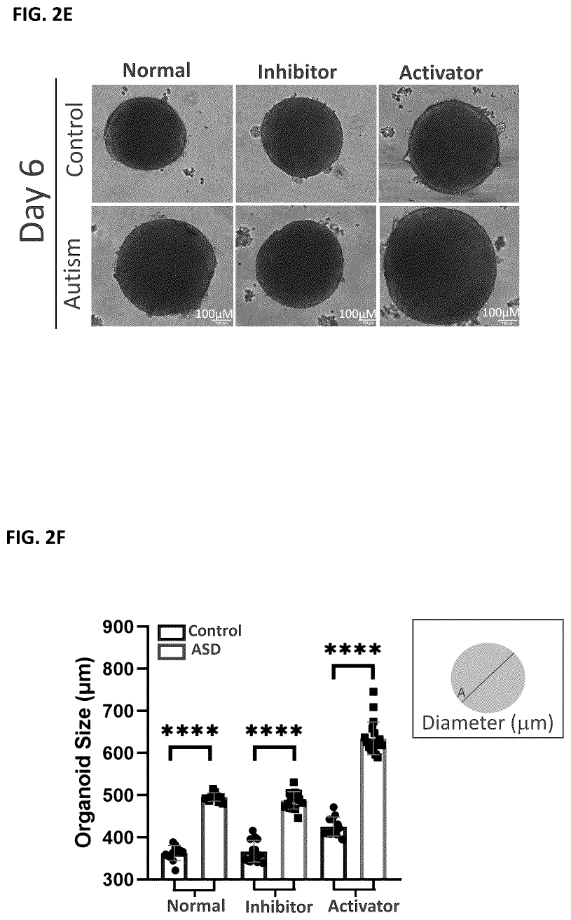

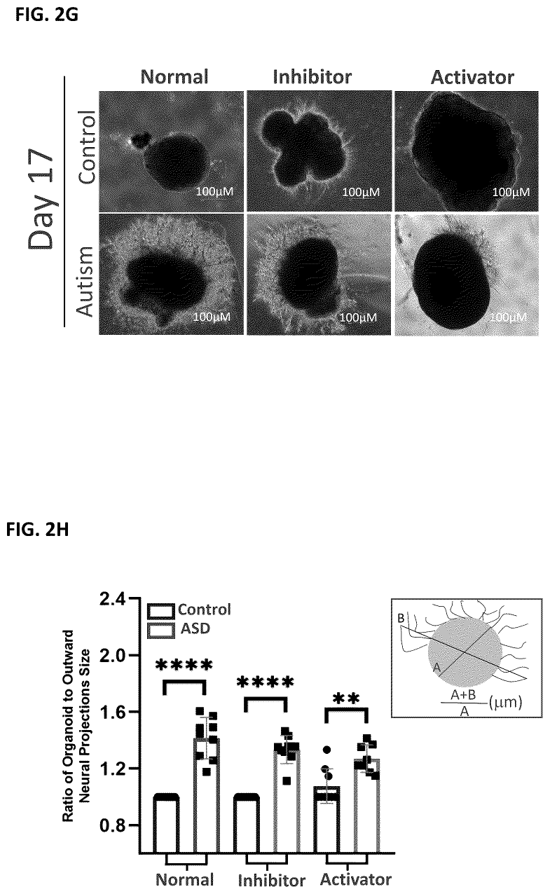

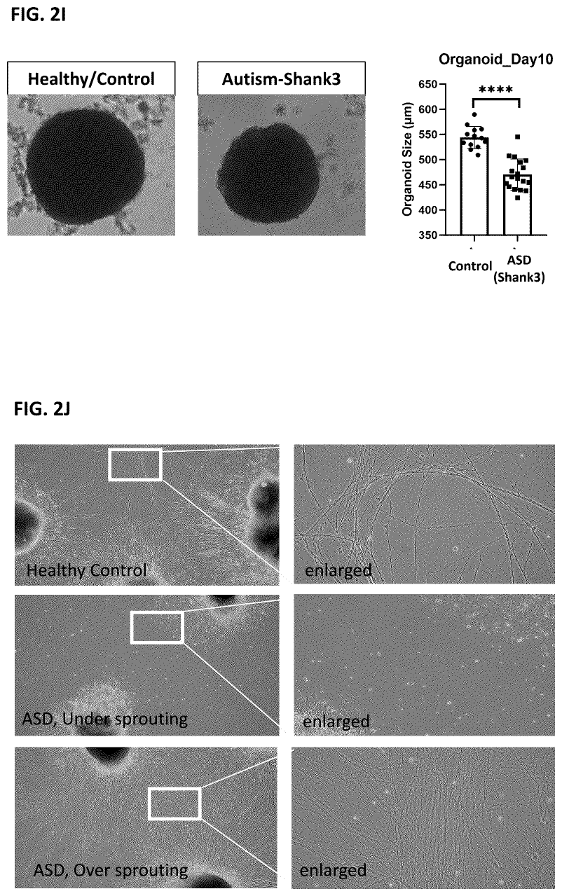

Reference is now made to Example 1, FIGS. 2A-2J, presenting visual attributes related to dendritic structure.

According to some embodiments, the one or more attribute(s) is a visual attribute.

According to some specific embodiments, the one or more attribute(s) include a deviation in the shape of dendritic projections of ASD-derived organoid relative to healthy organoid.

In some specific embodiments, differences in the shape of dendritic projections include increment or reduction in a grainy appearance of the projection. Each possibility is a separate embodiment.

According to some specific embodiments, the one or more attribute(s) include a deviation in spine density along dendritic projections of ASD-derived organoid relative to healthy organoid.

According to some specific embodiments, the one or more attribute(s) include a deviation in the number of spines along dendritic projections of ASD-derived organoid relative to healthy organoid.

In some specific embodiments, differences in the density of spines along dendritic projections include increment or reduction in the number of spines along the dendritic projections. Each possibility is a separate embodiment.

In some specific embodiments, differences in the density of spines along dendritic projections include increment in the number of spines along the dendritic projections. Each possibility is a separate embodiment.

According to related embodiments, the one or more visual attribute(s) includes a structure or shape of dendritic projections including dendritic spines. Each possibility is a separate embodiment.

In some embodiments, the assessing of the attribute of dendritic projections structure/shape includes determining one or more of spine density along the dendritic projection, spine shape, and spine size/volume, or any combination thereof. Each possibility is a separate possibility.

In some specific embodiments, the assessing of the attribute of dendritic projections structure/shape includes determining spine density along the projection.

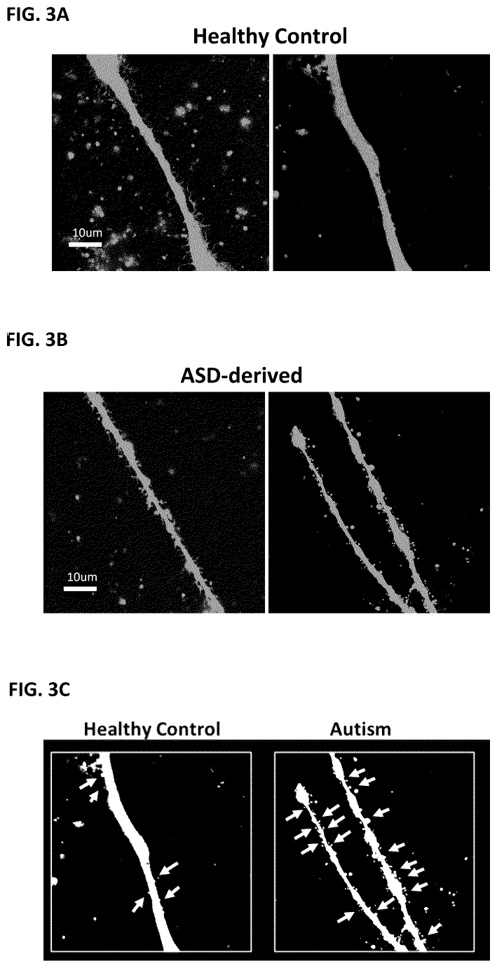

Reference is now made to Example 2, FIGS. 3A-3C, presenting visual attributes related to brain organoid structure.

According to other embodiments, the one or more signal(s) and/or attribute(s) includes electrical activity; and wherein the electrical activity is indicative of neuronal function. Each possibility is a separate possibility.

In some embodiments, the electrical activity signal includes electrophysiological measurements and/or activity reporter imaging. Each possibility is a separate possibility.

In some embodiments, the electrical activity indicative of neuronal function includes electrical activity signal recorded from the brain organoid in response to one or more treatment/stimuli sessions, each session includes electrophysiological stimuli provided to the brain organoid. Each possibility is a separate embodiment.

In some embodiments, the electrical activity indicative of neuronal function includes electrical activity recorded in response to pre-treatment with electrophysiological stimuli.

According to some embodiments, the assessing of the one or more signal(s) of electrical activity includes determining a behavior of the brain organoid based on the electrical activity recorded in response to the one or more treatment/stimuli session(s). Each possibility is a separate embodiment.

In some embodiments, the assessing includes determining a degree of similarity between the determined behavior of the prenatal or neonatal brain organoid and behavior of PD-derived organoids, or between the determined behavior of the prenatal or neonatal brain organoid and behavior of a healthy organoid;

In some embodiments, the behavior of the PD-derived organoids and the healthy organoids is predetermined/predicted with respect to the same electrophysiological stimuli provided to the brain organoids in the one or more treatment/stimuli session(s). Each possibility is a separate embodiment.

In some specific embodiments, the treatment/stimulus provided to the brain organoid in the one or more session(s) are fixed/predetermined (open loop). Each possibility is a separate embodiment.

In some embodiments, the AI algorithm is trained on brain-organoids behaviors of a plurality of healthy and/or PD derived brain organoids in response to the predetermined treatment/stimulus, wherein the training data is labeled according to one or more predetermined parameters of the treatment/stimulus (open loop). Each possibility is a separate embodiment.

According to some embodiments, the AI algorithm is continuously reinforced, based on the determined brain-organoid behavior, to thereby improve the predicted behavior (open and closed loop).

In some specific embodiments, the stimulus provided to the brain organoid is determined according to the determined brain-organoid behavior. (closed loop)

In some specific embodiments, the electrophysiological stimulus provided to the brain organoid in a latter session is determined based on the brain organoids' behavior determined in response to one or more former stimuli sessions (closed loop). Each possibility is a separate embodiment.

In a further specific embodiment, the electrophysiological stimulus provided to the brain organoid is positive or negative feedback stimuli. Each possibility is a separate embodiment.

In some embodiments, a change in the brain (closed loop) organoids behavior between a former and the latter sessions is indicative of a learning-behavior response of the brain organoid (closed loop).

According to some embodiments, the AI algorithm is a reinforced learning algorithm trained on brain organoids behaviors of a plurality of healthy and/or PD derived brain organoids, wherein the training data is labeled according to one or more parameters of the positive of negative feedback treatment/stimulus (closed loop).

According to some embodiments, the obtained brain organoid includes 3D brain organoid in culture.

According to some related embodiments, the obtained brain organoid includes tissue and/or cells thereof in 2D culture, and wherein the tissue and/or cells include sliced tissue and/or dissociated cells resulted from any of enzymatic, chemical, and/or mechanical processing of a 3D brain organoid. Each possibility is a separate embodiment.

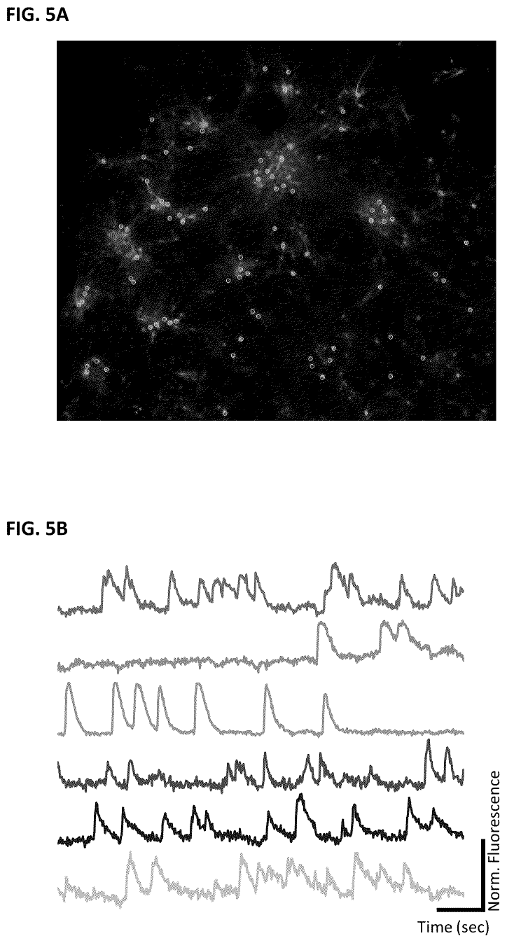

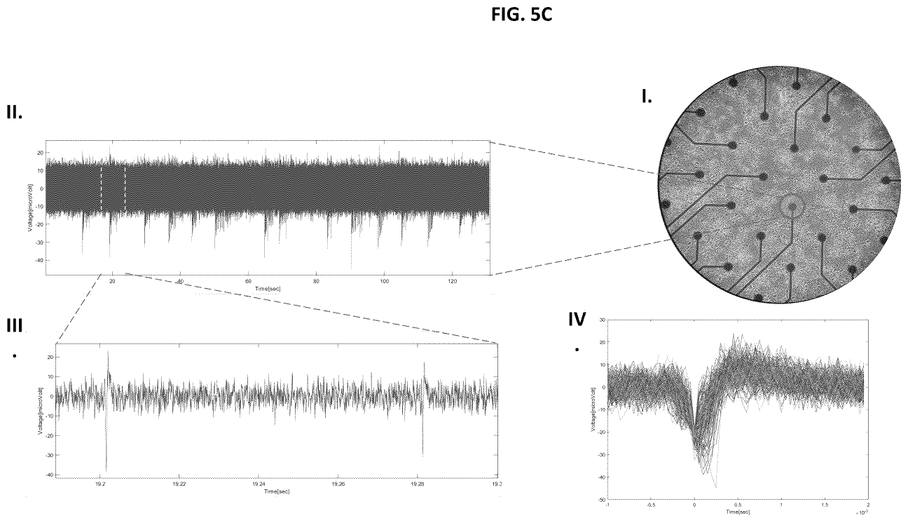

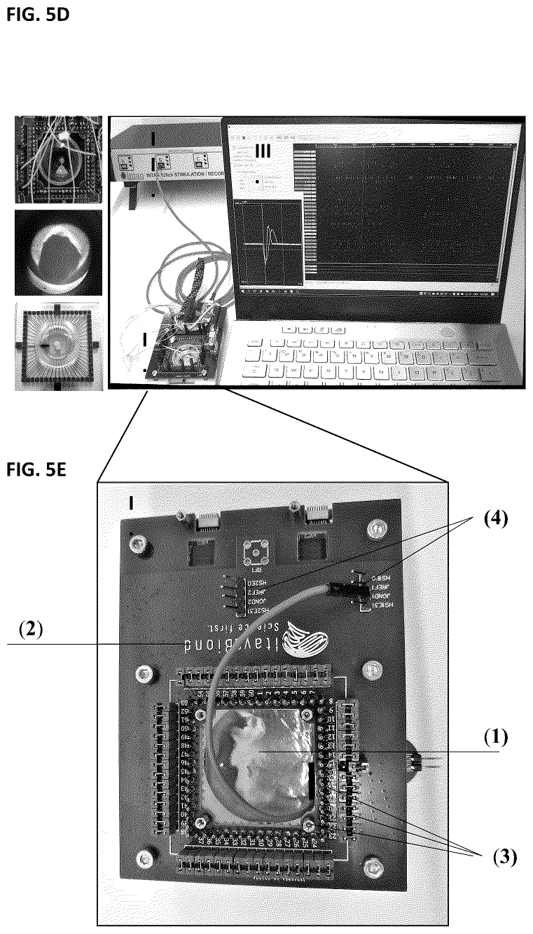

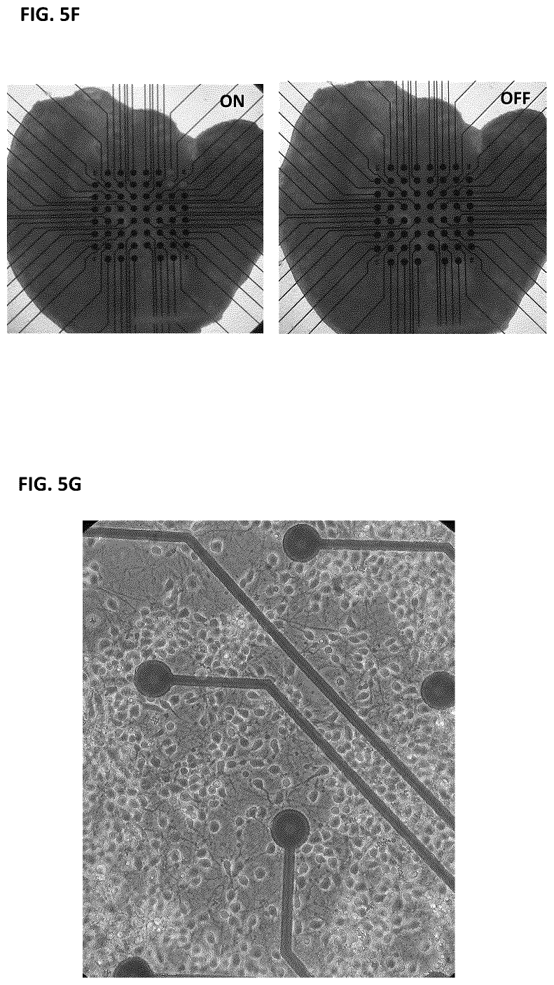

Reference is now made to Examples 4-6, FIGS. 5A-5G, FIGS. presenting electrophysiological recordings.

In some embodiments, the method further includes repeatedly providing stimuli sessions to the organoid, obtaining data recorded in response to each session, and determining the brain-organoid behavior based on the recorded data, after treatment of the organoid with a PD medicament including neurological, and/or neurodevelopmental medicaments.

In some embodiments, the PD medicament includes neurological, neurodegenerative, and/or neurodevelopmental medicaments.

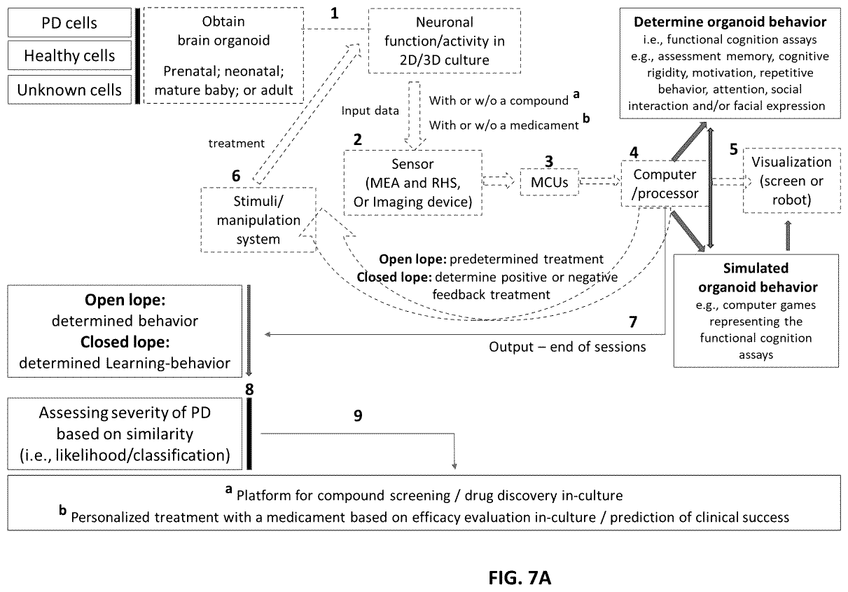

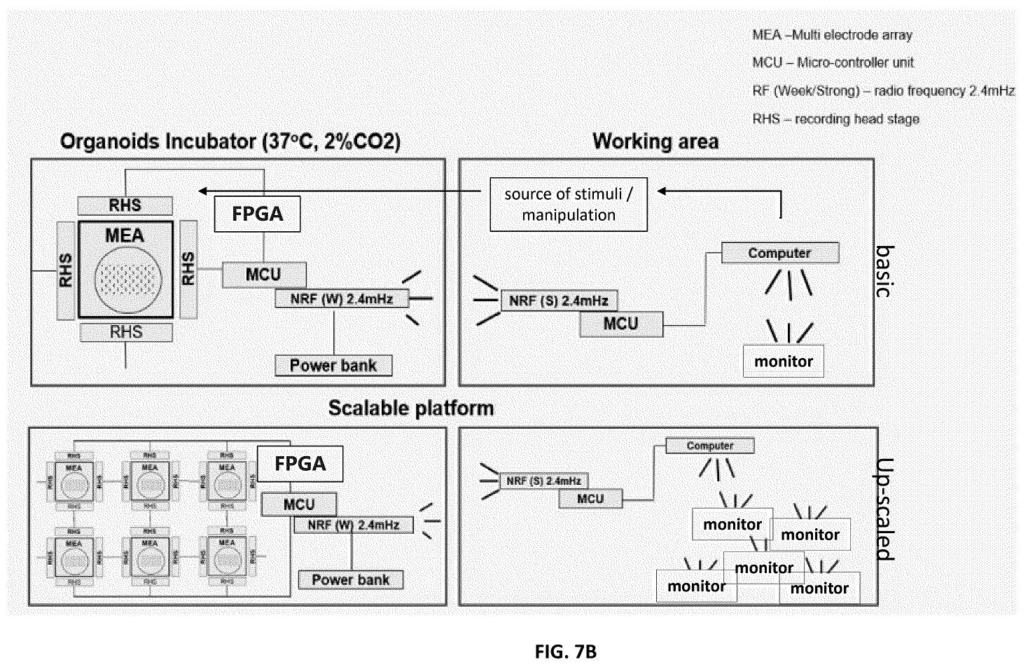

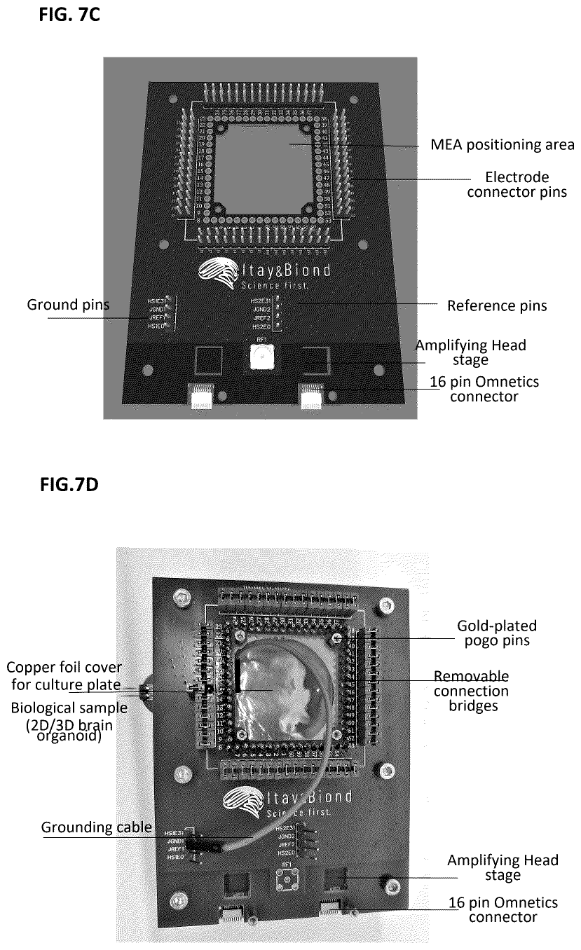

Reference is made to Examples 7 and FIG. 7A describing the use of the system for personalized evaluation of treatment efficacy with a medicament.

In some embodiments, the brain organoid is an undetermined brain organoid having unknown severity of PD.

According to some embodiments, the assessment of one or more signal(s) and/or attribute(s) further includes assessing gene expression and genomic abnormalities. Each possibility is a separate embodiment.

According to another aspect, the invention provides a system for prenatal and/or neonatal assessment of psychiatric disorder (PD), the system comprising:
(i) a prenatal or neonatal-derived brain organoid; (ii) one or more sensor(s) coupled to a recorder capable of detecting and archiving/recording one or more signal(s) and/or attribute(s) of the prenatal or neonatal-derived brain organoid; (iii) a micro-controller unit (MCU) configured to receive, integrate and/or transmit information/data of the one or more signal(s) and/or attribute(s); (iv) processor configured to: (a) obtain from the MCU the recorded data of the one or more signal(s) and/or attribute(s), and (b) assessing the one or more signal(s) and/or attribute(s) based on the data.

In some embodiments, the assessing includes determining a degree of similarity between the one or more signal(s) and/or attribute(s) of the prenatal or neonatal brain organoid and PD-derived organoids, or between the one or more signal(s) and/or attribute(s) of the prenatal or neonatal brain organoid and healthy organoids. Each possibility is a separate embodiment.

In some embodiments, the processor is configured to classify the prenatal or neonatal organoid as ‘PD’ or ‘healthy’ based on the degree of similarity.

In some related embodiments, the computer/processor is further configured to: apply an AI algorithm on the data of the one or more signal(s) and/or attribute(s) to thereby classifying the brain organoid based on a degree of similarity of the one or more signal(s) and/or attribute(s) of the organoid to PD-derived organoids or healthy organoids; and wherein the classification includes classifying the organoid according to ‘severity of PD’ or ‘healthy’.

In some related embodiments, the AI algorithm is trained on data of one or more signal and/or attribute of a plurality of healthy and/or PD derived brain organoids. Each possibility is a separate embodiment.

According to some embodiments, the one or more attribute(s) include a visual external phenotype of the organoid and/or neural projections thereof selected from one or more of: tissue morphology or 3D structure, organoid shape, organoid size, dendritic projection shape, spine density, or any combination thereof. Each possibility is a separate embodiment.

In some embodiments, the sensor includes an imaging device capable of visualizing the external phenotypes of the organoid and/or neural projections thereof. Each possibility is a separate embodiment.

In some embodiments, an imaging device includes, for example, but is not limited to illumination and contrast methods, and fluorescence methods.

According to some specific embodiments, the one or more attribute(s) include organoid shape and/or size. Each possibility is a separate embodiment.

According to some embodiments, the one or more attribute(s) include a visual external phenotype of the organoid and/or neural projections thereof; and wherein the visual phenotype is selected from one or more of: organoid shape, organoid size, dendritic projection shape, spine density, or any combination thereof. Each possibility is a separate embodiment.

According to some embodiments, the one or more attribute(s) include a visual external phenotype of the organoid and/or neural projections thereof; and wherein the visual phenotype includes one or more of: organoid shape, organoid size, dendritic projection shape, spine density, or any combination thereof. Each possibility is a separate embodiment.

According to some embodiments, the one or more attribute(s) include a visual external phenotype of the organoid and/or neural projections thereof; and wherein the visual phenotype includes one or more of: organoid shape, organoid size, and spines density, or any combination thereof. Each possibility is a separate embodiment.

According to some embodiments, the one or more attribute(s) is a visual attribute.

According to some embodiments, the one or more signal(s) and/or attribute(s) from visual external phenotype and/or electrophysiological response/signal. Each possibility is a separate embodiment.

In some embodiments, the visual external phenotype includes the organoid and/or neural projections thereof. Each possibility is a separate embodiment.

In some embodiments, the electrophysiological response/signal includes determining an organoid behavior.

According to some embodiments, the one or more signal(s) and/or attribute(s) is selected from one or more of: organoid shape, organoid size, dendritic projection shape, spine density, and electrophysiological response/signal, or any combination thereof. Each possibility is a separate embodiment.

In some specific embodiments, the one or more visual attribute(s) include a deviation in the size of an ASD-derived organoid from a healthy organoid.

In some embodiments, deviation in the size of an organoid includes increment or reduction in the size. Each possibility is a separate embodiment.

In some specific embodiments, organoid size includes organoid diameter (A) (μm). In some specific embodiments, organoid size includes a ratio between the organoid diameter (A) (μm) and its outwards neural projection (B) (μm).

In some embodiment, the attribute of organoid size comprises a deviation in a mean organoid diameter (A) of a PD-derived organoid from a mean organoid diameter of a healthy organoid; wherein a deviation includes increment or decrease in the diameter. Each possibility is a separate embodiment.

In some embodiments, the deviation in a mean organoid diameter (A) of a PD-derived organoid from a mean organoid diameter (A) of a healthy organoid includes increment in the size. Each possibility is a separate embodiment.

In some embodiments, the deviation in a mean organoid diameter (A) of a PD-derived organoid from a mean organoid diameter (A) of a healthy organoid includes decrease in the size. Each possibility is a separate embodiment.

In some embodiment, the attribute of organoid size includes a deviation in a mean ratio between the organoid diameter (A) (μm) and its outwards neural projection (B) (μm) of a PD-derived organoid from a mean ratio of a healthy organoid; wherein the deviation includes increment or decrease in the ratio. Each possibility is a separate embodiment.

In some embodiments, the deviation in a mean ratio between the organoid diameter (A) (μm) and its outwards neural projection (B) (μm) of a PD-derived organoid from a mean ratio of a healthy organoid includes increment in the ratio. Each possibility is a separate embodiment.

In some embodiments, the deviation in a mean ratio between the organoid diameter (A) (μm) and its outwards neural projection (B) (μm) of a PD-derived organoid from a mean ratio of a healthy organoid includes decrease in the ratio. Each possibility is a separate embodiment.

In some specific embodiments, the one or more visual attribute(s) include a deviation in the shape of ASD-derived organoid relative to healthy organoid.

In some specific embodiments, differences in the shape of an organoid include increment or reduction in the amount/expression of projection (over or under sprouting). Each possibility is a separate embodiment.

In some specific embodiments, differences in the shape of an organoid include increment or reduction in the amount/expression of projection (over or under sprouting). Each possibility is a separate embodiment.

In some embodiments, the deviation in the amount/expression of neural projections of a PD-derived organoid from the amount/expression of neural projections of a healthy organoid; includes increment or decrease in the amount of sprouting. Each possibility is a separate embodiment.

In some embodiments, the deviation in the amount/expression of neural projections of a PD-derived organoid from the amount/expression of neural projections of a healthy organoid; includes increment in the amount of sprouting. Each possibility is a separate embodiment.

In some embodiments, the deviation in the amount/expression of neural projections of a PD-derived organoid from the amount/expression of neural projections of a healthy organoid; includes decrease in the amount of sprouting. Each possibility is a separate embodiment.

As used herein the term “neural projections” and “sprouting” may be interchangeably used.

In some embodiments, assessing the attribute of organoid shape includes assessing over-expression or under-expression of neural projections. Each possibility is a separate embodiment.

According to some specific embodiments, the one or more visual attribute(s) include a deviation in the shape of dendritic projections of ASD-derived organoid relative to healthy organoid.

In some specific embodiments, differences in the shape of dendritic projections include increment or reduction in a grainy appearance of the projection. Each possibility is a separate embodiment.

According to some specific embodiments, the one or more visual attribute(s) include a deviation in spine density along dendritic projections of ASD-derived organoid relative to healthy organoid.

According to some specific embodiments, the one or more visual attribute(s) include a deviation in the number of spines along dendritic projections of ASD-derived organoid relative to healthy organoid.

In some specific embodiments, differences in the density of spines along dendritic projections include increment or reduction in the number of spines along the dendritic projections. Each possibility is a separate embodiment.

In some embodiments, the attribute of spines density comprises a deviation in an amount/number of spines along a dendritic projection of a PD-derived organoid from an amount/number of spines along a dendritic projection of healthy organoid, wherein the deviation includes increment or decrease in the density.

In some embodiments, a deviation includes an increment in the density of spines.

In some embodiments, the attribute of spines density comprises a deviation in an amount/number of spines along a dendritic projection of a PD-derived organoid from an amount/number of spines along a dendritic projection of healthy organoid, wherein the deviation includes increment in the density.

In some specific embodiments, the assessing of the attribute of organoid size includes determining organoid diameter (A) (μm).

In some related specific embodiments, the assessing of the attribute of organoid shape and/or size includes determining a ratio between the organoid diameter (A) (μm) and its outwards neural projection (B) (μm). Each possibility is a separate embodiment.

In some further specific embodiments, the ratio is calculated as ((A+B)/A) (μm).

According to some embodiments, the one or more attribute(s) includes a structure or shape of dendritic projections including dendritic spines. Each possibility is a separate embodiment.

In some embodiments, the assessing of the attribute of dendritic projections structure/shape includes determining one or more of spine density along the dendritic projection (number per area of the projection), spine shape, and spine size/volume, or any combination thereof. Each possibility is a separate embodiment.

In some embodiments, the one or more attribute(s) includes increased spines density. In some embodiments, the one or more attribute(s) includes increased number of spines per projection.

According to some embodiments, the one or more signal(s) includes electrical activity; and wherein the electrical activity is indicative of neuronal function.

In some specific embodiments, the electrical activity includes electrophysiological measurements and/or activity reporter imaging. Each possibility is a separate embodiment.

Reference is made to FIGS. 5A-5E and FIG. 7B

According to some embodiments, the system further includes a stimuli system capable of delivering stimuli to the prenatal or neonatal brain organoid; and wherein the processor is further configured to send instructions to the stimuli system to provide one or more treatment/stimuli sessions, each session includes electrophysiological stimuli provided to the brain organoid.

In some embodiments, each session includes a treatment.

In some embodiments, the sensor includes a multi-electrode array (MEA) coupled to one or more recording head stage (RHS) capable of recording electrical activity from the brain organoid.

In some related embodiments, the system includes two different types of sensors, including for example an imaging device and a multi-electrode array (MEA). In some related embodiments, the system includes an imaging device and at least an additional sensor. In some related embodiments, the system includes a multi-electrode array (MEA) and at least an additional sensor.

In some related embodiments, the system includes two sensors, a first sensor including an imaging device capable of visualizing the external phenotypes of the organoid and/or neural projections thereof, and a second sensor including a multi-electrode array (MEA) coupled to one or more recording head stage (RHS) capable of recording electrical activity.

In some embodiments, the electrical activity indicative of neuronal function includes electrical activity signal recorded from the brain organoid in response to the one or more treatment/stimuli session(s) including the electrophysiological stimuli provided to the brain organoid.

In some embodiments, the computer/processor is further configured to determine a behavior of the brain organoid based on the electrical activity signal recorded in response to the one or more treatment/stimuli session(s). In some embodiments, the one or more attribute(s) includes a brain organoid behavior.

In some embodiments, the computer/processor is further configured to apply an AI algorithm on the brain organoid behavior to thereby classify the brain organoid based on a degree of similarity of the determined behavior of the brain organoid to a predicted behavior of a PD-derived brain organoids and/or healthy organoids; and wherein the classification includes classifying the organoid according to ‘severity of PD’ or ‘healthy’.

In some embodiments, the treatment/stimulus provided to the brain organoid in the one or more session(s) are fixed/predetermined.

In some embodiments, the AI algorithm is a algorithm trained on brain-organoids behaviors of a plurality of healthy and/or PD derived brain organoids in response to the predetermined stimulus, wherein the training data is labeled according to one or more parameters of the treatment/stimulus. Each possibility is a separate embodiment. (open loop)

In some embodiments, the treatment/stimulus provided to the brain organoid in a latter session is determined based on the brain organoid's behavior determined in response to one or more former stimuli sessions.

In some embodiments, the AI algorithm is a reinforced learning algorithm trained on brain-organoids behaviors of a plurality of healthy and/or PD derived brain organoids, wherein the training data is labeled according to one or more parameters of the treatment/stimulus. Each possibility is a separate embodiment. (closed loop)

In some embodiments, the treatment/stimulus provided to the brain organoid is positive or negative feedback stimuli; and wherein a change in the brain-organoids behavior between a former and the latter sessions is indicative of a learning behavior response of the brain organoid. Each possibility is a separate embodiment. (closed loop)

In some embodiments, the classifying of the brain organoid is based on a degree of similarity of the learning-behavior response to a predicted learning-behavior response of a PD-derived brain organoid and/or of a healthy organoid (closed loop). Each possibility is a separate embodiment.

Reference is made to Example 8, FIG. 7A-7B illustrating the system.

According to some further embodiments, the processor is configured to generate a visual simulation representative of the determined organoid behavior.

In some embodiments, the visual simulation includes a computer game representing the evaluation of cognitive abilities or functionality of neuronal network. Each possibility is a separate embodiment.

In some embodiments, the computer game is configured to evaluate cognitive abilities selected from one or more of: memory, cognitive rigidity, motivation, repetitive behavior, attention, social interaction, processing speed, executive function, numerical abilities, and/or facial expression, or any combination thereof. Each possibility is a separate embodiment.

In some embodiments, the visualization component is configured to present evaluation of cognitive abilities selected from one or more of: memory, cognitive rigidity, motivation, repetitive behavior, attention, social interaction, processing speed, executive function, numerical abilities, and/or facial expression, or any combination thereof. Each possibility is a separate embodiment.

According to some embodiments, the obtained brain organoid includes3D brain organoid in culture.

According to some embodiments, the obtained brain organoid includes tissue and/or cells thereof in 2D culture, and wherein the tissue and/or cells include sliced tissue and/or dissociated cells resulted from any of enzymatic, chemical, and/or mechanical processing of a 3D brain organoid. Each possibility is a separate embodiment.

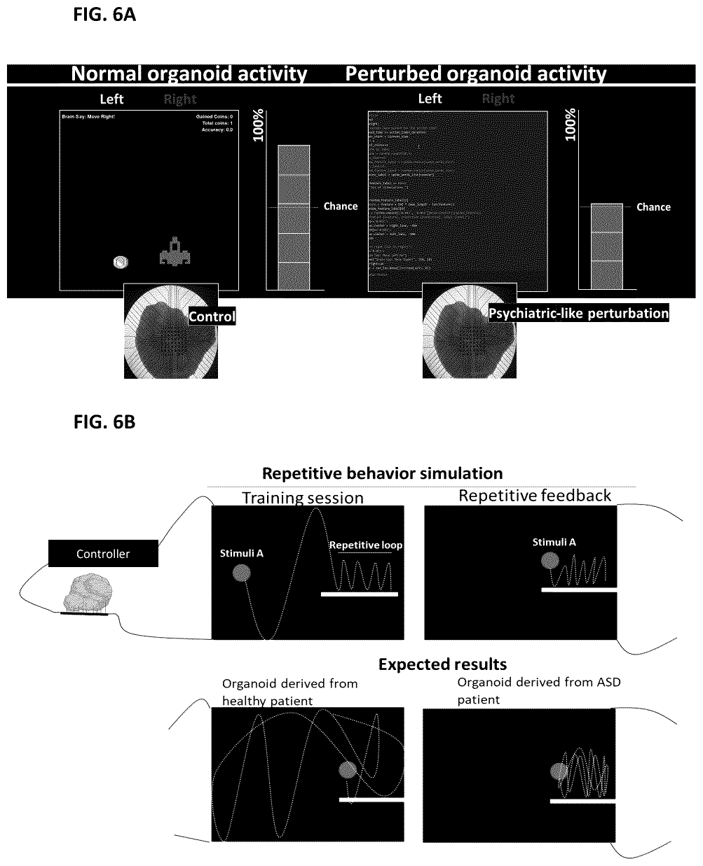

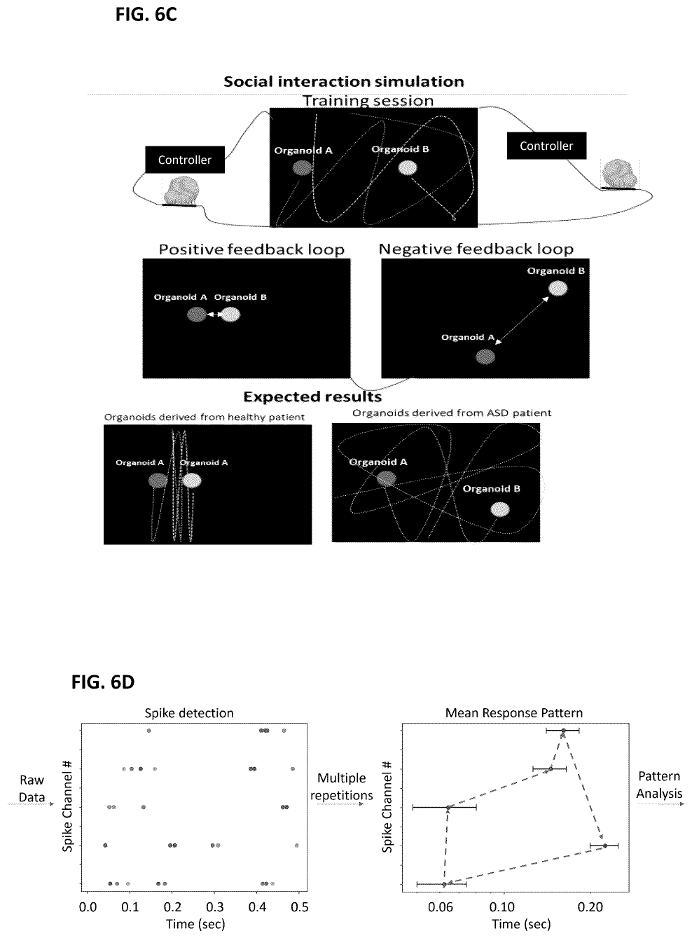

Reference is made to Examples 4-6, FIGS. 5A-5C, FIGS. 6A-6D presenting electrophysiological recordings and visual simulations.

In some embodiments, the processor is further configured to repeatedly providing stimuli sessions to the organoid, obtaining data recorded in response to each session, and determining the brain-organoid behavior based on the recorded data, after treatment of the organoid with a PD medicament including neurological and/or neurodevelopmental medicaments. Each possibility is a separate embodiment.

In some embodiments, the processor is further configured to determine the efficacy of the treatment.

Reference is made to Example 7 and FIG. 7A describing the use of the system for personalized evaluation of treatment efficacy with a medicament.

In some embodiments, the brain organoid is an undetermined brain organoid having unknown severity of PD.

According to an additional aspect, the invention provides a prenatal or neonatal brain organoid for assessment of a psychiatric disorder (PD) wherein the organoid is generated from human prenatal or neonatal cells, and wherein the assessment includes assessment of one or more signal(s) and/or attribute(s).

According to some embodiments, the PD includes one or more of Autism Spectrum Disorders (ASD), Bipolar disorder, Attention Deficit Hyperactivity Disorder (ADHD/ADD), Schizophrenia, Depression, Obsessive-Compulsive Disorders (OCD), Epilepsy, or any combination thereof. Each possibility is a separate embodiment.

In some embodiments, the PD includes non-genetic psychiatric disorder (non-genetic PD).

In some embodiments, the assessing of one or more signal and/or attribute is with respect to healthy organoids.

In some embodiments, the one or more attribute(s) includes visual external phenotypes of the organoid and/or neural projections thereof selected from one or more of: cell/tissue morphology, 3D structural elements, shape and/or size, or any combination thereof. Each possibility is a separate embodiment.

In some embodiments, the one or more attribute(s) includes the organoid's size or shape. In some embodiments, the one or more attribute(s) includes the organoid's size.

In some embodiments, the size attribute includes the organoid's diameter (A) (μm).

In some embodiments, the one or more attribute(s) includes a ratio between the organoid diameter (A) (μm) and its outwards neural projection (B) (μm).

In some embodiments, the ratio is calculated as ((A+B)/A) (μm).

In some embodiments, the one or more attribute(s) includes the structure or shape of the dendritic projections including dendritic spines.

In some embodiments, the one or more signal(s) includes electrical activity; and wherein the electrical activity is indicative of neuronal function recorded from the brain organoid in response to one or more treatment/stimuli.

In some embodiments, the treatment/stimuli include electrophysiological stimuli provided to the brain organoid.

In some embodiments, the one or more attribute(s) includes organoid behavior determined based on electrical activity recorded from the brain organoid in response to one or more treatment/stimuli.

According to some embodiments, the invention provides a method for training an AI algorithm for determining organoids behavior, the method comprising:
(a) obtaining a plurality of PD-derived brain organoid and a plurality of healthy brain organoid; (b) providing one or more stimuli sessions, each session including a stimuli provided to the brain organoid; (c) obtaining data recorded in response to the one or more treatment/stimuli session(s), the data is indicative of neuronal function/activity of the brain organoid; (d) labeling the data according to parameters of the one or more stimuli session(s) and associating the labeled data with the PD-derived brain organoid and/or with the plurality of healthy brain organoid; (e) applying an AI algorithm on the data to learn patterns and relationships and to adjust parameters of a model for organoid behavior prediction; thereby training the algorithm for determining a brain-organoids behavior based on the data recorded in response to the one or more treatment/stimuli session(s).

In some embodiments, the AI algorithm is further trained to classify the organoids plurality of PD-derived brain organoids and/or healthy organoids based on the determined organoids' behavior as having ‘PD-derived behavior’ or a ‘healthy behavior’; thereby classifying the brain organoids based on a degree of similarity of their determined behavior to a predicted behavior of a PD-derived brain organoid and/or a healthy organoid.

In some embodiments, the obtaining of PD-derived brain organoid includes organoids having a range of PD severities, and wherein the association of the labeled data with the PD-derived brain organoid includes associating the labeled data with the range of PD severities; thereby augmenting the prediction behavior model to include a range of severities.

In some embodiments, the data indicative of neuronal function/activity of the brain organoid is divided to a ‘training dataset’ and ‘validation set’, and wherein the ‘validation set’ includes unlabeled data used to improve model performance.

In some embodiments, the AI algorithm includes supervised learning, unsupervised learning, semi-supervised learning, and reinforced learning, or any combination thereof. Each possibility is a separate embodiment.

In some embodiments, the AI algorithm includes a supervised machine learning algorithm capable of regression and/or classification, including for example, but not limited to one or more of: Support-vector machines, Linear regression, Logistic regression, Naive Bayes, Linear discriminant analysis, Decision trees, K-nearest neighbor algorithm, Neural networks (Multilayer perceptron), Similarity learning, or any combination thereof. Each possibility is a separate embodiment.

Other non-limiting examples of supervised machine learning algorithm capable of regression and/or classification, including for example, but not limited to: Analytical learning, Artificial neural network, Backpropagation, Boosting (meta-algorithm), Bayesian statistics, Case-based reasoning, Decision tree learning, Inductive logic programming, Gaussian process regression, Genetic, programming, Group method of data handling, Kernel estimators, Learning automata, Learning classifier systems, Learning vector quantization, Minimum message length (decision trees, decision graphs, etc.), Multilinear subspace learning, Naive Bayes classifier, Maximum entropy classifier, Conditional random field, Nearest neighbor algorithm, Probably approximately correct learning (PAC) learning, Ripple down rules, a knowledge acquisition methodology, Symbolic machine learning algorithms, Subsymbolic machine learning algorithms, Support vector machines, Minimum complexity machines (MCM), Random forests, Ensembles of classifiers, Ordinal classification, Data pre-processing, Handling imbalanced datasets, Statistical relational learning, Proaftn, a multicriteria classification algorithm.

In some embodiments, the training includes open loop training mode, wherein the treatments/stimuli provided to the brain organoid in the one or more session(s) are predetermined.

In some embodiments, the training includes closed loop training mode, wherein the treatments/stimuli provided to the brain organoid in the one or more session(s) is determined according to the determined brain-organoid behavior.

In some embodiments, at least the plurality of healthy brain organoids is generated from prenatal or neonatal cells.

In some embodiments, at least the plurality of PD-derived brain organoid is generated from prenatal or neonatal cells.

In some embodiments, the training of the AI algorithm for determining organoids behavior includes determining prenatal and/or neonatal organoids behavior.

According to some embodiments, the invention provides a method for training an AI algorithm for determining one or mor signal(s) and/or attribute(s), the method comprising:
(a) obtaining a plurality of PD-derived brain organoid and a plurality of healthy brain organoid; (b) obtaining data recorded of one or mor signal(s) and/or attribute(s) labeling the data according to parameters of the one or mor signal(s) and/or attribute(s) and associating the labeled data with the PD-derived brain organoid and/or with the plurality of healthy brain organoid; (c) applying an AI algorithm on the data to learn patterns and relationships and to adjust parameters of a model; thereby training the algorithm for determining one or mor signal(s) and/or attribute(s). Each possibility is a separate embodiment.

The term “assessing/assessment”, refers to diagnosis PD, i.e., classification of a brain organoid as PD or healthy, and further evaluation of the severity of PD. This is achieved by comparing the similarity of the determined brain organoids behavior to predicted behaviors of plurality of healthy or PD-derived organoids. The assessment includes the organoids neural network response, or determining a behavior based on the neural network response to treatment/stimuli provided to the organoid or 2D cultures derived therefrom, during one or more stimuli sessions.

The systems and methods provide a platform for evaluation of PD based on an interactive process between a brain organoid and components of the system in a closed loop or open loop modes, and including a source of stimuli, a sensor, and a processor that is configured to execute neuro-computational simulations that include providing stimuli and determining responses. in an iterative process of reinforcement learning in neurons of the brain organoid that ultimately encompass a measure for the computational and high-order functions of the brain organoid.

For example, in the closed loop mode, this may be achieved by repeatedly determining positive or negative feedback (i.e., neural behavior/function) based on computer-simulated information indicative of neuronal function/activity in response to preceding feedback, computing the change in response (i.e., the process of reinforcement of learning), and scoring it according to predicted response determined by AI algorithm trained in a similar process of determining learning-behavior response performed on a PD-derived brain organoid and/or a healthy brain organoid, thereby evaluating the severity of PD.

Therefore, the assessment of PD using the closed loop is performed by determining a learning-behavior response driven/mediated by the interplay between a brain organoid and components of the system that repeatedly stimulate and simulate it.

As used herein the term, “behavior”, is related to the term “neuronal function/activity” and may refer to the response of the neural network or to patterns or relationships that underly the neural network response to stimuli. The behavior response is characteristic of the function/activity of the brain organoid, and 2D neuronal cultures thereof, with respect to those specific stimuli they were exposed to. The behavior/response refers to the data/information that was recorded in response to the stimuli provided during one or mor stimuli sessions.

The behavior refers to a behavior/response determined in response to a process driven/mediated by the open or the closed loop mode.

In some embodiments, the behavior includes a learning-behavior (closed loop).

In some embodiments, brain organoid behavior includes data/information indicative of neuronal function/activity of the brain organoid.

The terms “behavior”, “behavior-like”, “response” and “behavior response”, “neuronal function/activity” and “simulated behavior” may be used interchangeably.

As used herein the term, “determined behavior”, may refer to the determined response of the neural network or to patterns or relationships that underly the neural network response to stimuli. Reference is made to the method of determining brain organoid behavior. The determined behavior response is characteristic of the function/activity of the brain organoid, and 2D neuronal cultures thereof, with respect to those specific stimuli they were exposed to, but it may reflect, a reduction in the data/information that was recorded, into a form where it can be classified as ‘PD-derived behavior’ or ‘healthy behavior’, or according to “PD-severity”.

In some embodiments, the learning of the patterns and relationships includes one or more of spatiotemporal propagation including duration or distribution of the signal, intensity, frequency, and amplitude, or any combination thereof.

In some embodiments, the learning of the patterns and relationships includes spatiotemporal propagation.

In some embodiments, determining the network response to stimuli includes the learning of the patterns and relationships of spatiotemporal propagation.

In some embodiments, determining a brain organoid behavior includes the learning of the patterns and relationships of spatiotemporal propagation.

Advantageously, based on the determined behavior, it may be, but not necessarily, easier to identify and predict a state of healthy or PD, than from the collective response/behavior of the network.

Also, a determined behavior is more prone to visualization through a visual simulation than the actual response of the network.

In some embodiments, the determined behavior comprises the response of the network to stimuli.

The term “simulated behavior” or “simulation” may be interchangeably used with the term “determined behavior” but may be more directed towards the whole process of recording data indicative of the network behavior/response, determining a behavior/response, and instructing to execute another stimuli session. The simulated behavior is the core process driven by the processor of the system/method that assesses the response of the network to one or more stimuli sessions.

In addition, the simulated behavior (or the determined behavior), may be visualized in a visual simulation using a simulation component such as a computer/screen/robot or the like.

The terms “determined behavior”, “determined response” and “simulated behavior” are interchangeable.

In some embodiments, simulating a behavior includes determining a behavior.

The terms “assessment of learning-behavior response associated with a psychiatric disorder (PD)”, “assessing PD”, and “assessment of the severity of a PD” may be interchangeably used.

According to some embodiments, assessment of a learning-behavior response associated with a PD-derived brain comprises assessing the severity of PD.

As used herein, the term “learning-behavior” refers to a learning process driven/mediated by the closed loop mode, involving stimuli and stimuli parameters, that are determined based on the organoid behavior determined in a former session(s) The learning process results from, and is reinforced by, repeatedly pairing a feedback stimulus with a preceding neural behavior and is herein computed as a change in the determined behavior/simulated behavior of a brain organoid (i.e., change in computational simulation of information indicative of the neuronal function/activity) in response to a positive or negative feedback treatment (i.e., feedback stimulus, elastic stimulus). The behavior response, including learning-behavior response is a computational functional analysis.

As used herein, the term “severity of PD” refers to assessment of a certain degree/level of PD. This is achieved by comparing the similarity of the determined brain organoids behavior to predicted behaviors of plurality of PD-derived brain organoids comprising organoids having a range of PD severities, thereby augmenting the determined behavior to include a range of severity, wherein the obtaining of PD-derived brain organoid comprises organoids having a range of PD severities, and wherein the association of the labeled data with the PD-derived brain organoid comprises associating the labeled data with the range of PD severities; thereby augmenting the prediction behavior model to include a range of severities.

The term “predicted behavior” refers to models of brain organoids behavior generated by the trained AI algorithm while learning a plurality of brain organoid responses to stimuli, provided thereto during one or more stimuli sessions. A predicted behavior may model a healthy-brain organoid behavior or PD-derived organoid behavior having a range of PD severities. (referring to the method of training).

As used herein, the term “data” is related to the term “signal” and may refer to any information indicative of neuronal function/activity of the brain organoid that is related to the signal detected or can be derived from it. In some embodiments, the information may include for example, but is not limited to: duration, intensity, frequency, amplitude, and/or spatial distribution/spatiotemporal propagation of the detected signal.

As used herein, the term “open loop” refers to a mode of the system for assessing PD wherein the algorithm learns and classifies the behavior of the brain in response to predetermined stimuli.

The term “predetermined stimuli” is related to the term open loop, and refers to a predetermined, fixed treatment/stimuli that does not depend on the response of the brain organoid to previous stimuli. The stimuli and its parameters may or may not repeat themselves between sessions, but they are predetermined/fixed in that sense that when the parameters are set it is done without considering the response of the brain organoid to previous stimuli. Hence, predetermined stimuli stand in contrast to elastic stimuli.

As used herein, the term “closed loop” refers to a mode of the system for assessing PD wherein the algorithm learns and classifies the learning-behavior of the brain in response to elastic stimuli.

As used herein, the term “elastic stimuli” is related to the term closed loop and refers to stimuli determined based on the organoid's behavior in one or more former sessions (e.g., positive or negative feedback stimuli).

In some embodiments, a behavior comprises a learning behavior.

In some embodiments, the determined brain-organoid behavior comprises a learning behavior.

As used herein, the term “behavior” or “brain-organoid behavior” refers to neural function/activity in response to stimuli.

As used herein, the term “labeled” or “labeling” may relate to the AI-training process used in a closed and open system/modes and may refer to one or more characteristics/parameters of the treatment/stimulus, including but not limited to stimulus type, stimuli pattern/distribution and other treatment parameters such as intensity, duration, amplitude, frequency, and the like.

In the open system/mode, the labeling includes association of one or more characteristics/parameters of the predetermined stimuli with an organoid behavior that correspond to a healthy or PD-derived organoid (including a spectrum of PD-derived organoid representing different levels of PD severities).

In the closed system/mode, the labeling includes association of one or more characteristics/parameters of the elastic stimuli with an organoid behavior that correspond to a healthy or PD-derived organoid (including a spectrum of PD-derived organoid representing different levels of PD severities).

As used herein, the term “similarity” refers to a comparison between a predicted behavior and a determined behavior.

As used herein, the term “long-term measurement” refers to an electrophysiological measurement performed by the system after a stimuli was delivered to the brain organoid, and persists for longer than 5 min. Non limiting example of a stimuli for long term measurements includes a drug or heat.

In some embodiments, a stimuli/treatment for long-term measurement one or more of electrophysiological stimuli, a drug, heat, light, or any combination thereof. Each possibility is a separate embodiment.

According to some embodiments, a long-term measurement persists for at least 5 min, at least 10 min, at least, 30 min, at least 60 min, at least 2 hours, at least 12 hours, at least 24 hours, at least several days, at least a week, at least several weeks, at least 1 month, at least 2 months, at least 6 months, or at least a year. Each possibility is a separate embodiment.

According to some embodiments, an electrophysiological measurement comprises at least a single ‘short term’ measurement of up to 5 min, at least two ‘short term’ measurements of up to 5 min each, at least three ‘short term’ measurements of up to 5 min each, at least four ‘short term’ measurements of up to 5 min each, at least five ‘short term’ measurements of up to 5 min each, at least ten ‘short term’ measurements of up to 5 min each. Each possibility is a separate embodiment.

According to some embodiments, an electrophysiological measurement comprises at least a single short-term measurement followed by a long-term measurement.

According to some embodiments, an electrophysiological measurement comprises at least a single long short term measurement, at least two consecutive cycles of long short term measurement, at least three consecutive cycles of long short term measurement, at least four consecutive cycles of long short term measurement, at least five consecutive cycles of long short term measurement, at least ten consecutive cycles of long short term measurement. Each possibility is a separate embodiment.

Referring to the principal components of the herein provided non-limiting illustration of which is presented in FIG. 7A-7B—The neural recording and stimulation setup described involves the following components:
Multi-Electrode Arrays (MEA): these arrays are used for recording and stimulating neural activity.
Headstage: This element has two non-limiting functions: 1. Converting analog neural signals to digital format for processing and vice versa. 2. Signal Processing function: including filtering, amplification, and optional basic Digital Signal Processing (DSP) capabilities for preprocessing the recorded neural data.
FPGA (Field-Programmable Gate Array) or Equivalent Element: This component is responsible for high-speed parallel signal processing tasks, such as spike detection and classification, as well as the synchronization of stimulation triggers.
Microcontroller Unit (MCU): The MCU executes various programs and tests. It is connected to the FPGA and can execute higher-level programming languages like Python and C. When dealing with a limited number of electrodes, the FPGA functions can be implemented in the MCU, and the FPGA can be removed, as extreme parallelism is not required for simple experiments.
Data Output and Control: The programs executed on the MCU can be used to control the setup and broadcast the output data over RF (Radio Frequency), wired connections, or store it on local disk storage.

In summary, this neural recording and stimulation setup comprises MEA arrays for neural signal acquisition, Headstage (signal processing and amplification), an FPGA (Field-Programmable Gate Array) or Equivalent Element for high-speed parallel processing, an MCU for program execution and control, and various options for data output and storage, Stimuli source and optionally additional computer/processor for additional analysis and visualization system. Depending on the complexity of the experiment and the number of electrodes used, the FPGA may be optional, with the MCU capable of handling simpler tasks.

According to one aspect, there is provided a system for assessment of a psychiatric disorder (PD), the system comprising:
(i) a brain organoid; (ii) a stimuli system capable of delivering stimuli to the brain organoid; (iii) a sensor coupled to a recorder capable of detecting and recording one or more signals indicative of neuronal function/activity of the brain organoid; (iv) a micro-controller unit (MCU) configured to receive, integrate and/or transmit data of the one or more signals; and (v) a computer/processor configured to:
(a) send instructions to the stimuli system to provide one or more stimuli sessions, each session comprising a stimuli provided to the brain organoid; (b) obtain from the MCU data recorded in response to the one or more stimuli sessions, the data indicative of neuronal function/activity of the brain organoid; (c) determine a brain-organoid behavior based on the recorded data; and (d) apply an AI algorithm on the brain-organoids behavior to thereby classify the brain organoid based on a degree of similarity of the determined brain-organoid behavior to a predicted behavior of a PD-derived brain organoid and/or a heathy organoid.

According to some embodiments, the system comprises an open loop, in which the stimulus provided to the brain organoid in the one or more sessions are predetermined.

In some embodiments, the AI algorithm is trained on brain-organoids behaviors of a plurality of healthy and/or PD derived brain organoids in response to the predetermined stimulus, wherein the training data is labeled according to one or more parameters of the stimulus. Each possibility is a separate embodiment. (open loop)

In some related embodiments, the AI algorithm is continuously reinforced, based on the determined brain-organoid behavior, to thereby improve the predicted behavior. (open loop)

According to some embodiments, the system comprises a closed loop, in which the stimulus provided to the brain organoid is determined according to the determined brain-organoid behavior. (closed loop)

In some embodiments, the AI algorithm is trained on brain-organoids behaviors of a plurality of healthy and/or PD derived brain organoids, wherein the training data is labeled according to one or more parameters of the treatment/stimulus. Each possibility is a separate embodiment. (closed loop)

In some specific embodiments, the processor is configured to instruct to the stimuli system to provide at least two sessions, wherein the stimuli provided in a latter session is determined based on the brain-organoids behavior determined in response to one or more former stimuli sessions. (closed loop)

In some further specific embodiments, the stimuli provided in a latter session comprises a positive or negative feedback; and wherein a change in the brain-organoids behavior between a former and the latter sessions is indicative of a learning behavior response of the brain organoid. Each possibility is a separate embodiment. (closed loop)

In some related embodiments, classifying the brain organoid is based on a degree of similarity of the learning-behavior response to a predicted learning-behavior response of a PD-derived brain organoid and/or of a heathy organoid. Each possibility is a separate embodiment. (closed loop)

In some embodiments, the system further comprises a visualization component presenting a visual simulation representative of the determined organoid behavior.

In some specific embodiments, the visual simulation comprises a computer game evaluating cognitive abilities selected from one or more of: memory, cognitive rigidity, motivation, repetitive behavior, attention, social interaction, processing speed, executive function, numerical abilities, and/or facial expression, or any combination thereof. Each possibility is a separate embodiment.

According to some embodiments, the processor is further configured to assess the severity of PD based on the similarity.

In another embodiment, the processor is further configured to repeat steps a-c on the brain organoid after treatment thereof with a neurological, neurodevelopmental and/or neurodegenerative medicament, or any combination thereof. Each possibility is a separate embodiment.

In yet another embodiment, the processor is further configured to repeat steps a-c on a brain organoid obtained from a same subject after neurological neurodevelopmental and/or neurodegenerative treatment of said subject, or any combination thereof. Each possibility is a separate embodiment.

In some specific embodiments, the neurological, neurodevelopmental and/or neurodegenerative treatment comprises a medicament. Each possibility is a separate embodiment.

In some embodiments, the processor is further configured to determine an efficacy of the treatment.

According to some embodiments, the brain organoid is derived from one or more of prenatal cells, neonatal cells, cells of a mature baby, cells of a toddler, cells of a child, cells of a teen, and cells of an adult, or any combination thereof. Each possibility is a separate embodiment.

In some embodiments, the brain organoid is an undetermined brain organoid having unknown severity of PD.

In some embodiment, the obtained brain organoid comprises 3D brain organoid in culture.

In related embodiments, the obtained brain organoid comprises tissue and/or cells thereof in 2D culture, and wherein the tissue and/or cells comprise sliced tissue and/or dissociated cells resulted from any of enzymatic, chemical, and/or mechanical processing of a 3D brain organoid, or any combination thereof. Each possibility is a separate embodiment.

In some embodiments, the sensor comprises one or more multi-array electrodes (MAE) coupled to one or more recording head stage (RHS).

In some embodiments, the stimuli system and the multi-array electrode (MAE) are same or different. Each possibility is a separate embodiment.

In some embodiments, the MCU is connected to a wireless radio transmitter (RF) or a micro transmitter (MT) connecting it to at least one remote MCU.

In some embodiments, the MCU is connected to a processor/computer or is an integral part thereof. Each possibility is a separate embodiment.

In related embodiments, at least the MAE, RHS and a plate holder for culturing the brain organoid are integrated in an all-in-one device.

In further related embodiments, the all-in-one device further comprises one or more of a stimuli system, an MCU and/or a processor, or any combination thereof. Each possibility is a separate embodiment.

According to some embodiments, the one or more signal indicative of the neuronal function/activity of the brain organoid comprises an electrophysiological signal; and wherein the sensor comprises MAE.

According to some embodiments, the one or more signal indicative of the neuronal function/activity of the brain organoid comprises a light signal of an activity reporter; and wherein the sensor comprises an imaging device.

In some embodiments, the data/information indicative of neuronal function/activity of the brain organoid comprises information of long-term measurements.

In some embodiments, the stimuli/treatment provided by stimuli system comprises one or more of electrophysiological stimuli, optic/light stimulus, heat, a chemical agent/drug, or any combination thereof. Each possibility is a separate embodiment.

In some embodiments, the stimuli/treatment provided by the stimuli system comprises electrophysiological stimuli.

According to some embodiments, at least some of the processing is done with a field-programmable gate array (FPGA).

In some embodiments, the data indicative of the neuronal function/activity comprises spatiotemporal propagation including spatial distribution and/or time after stimulation, intensity, frequency, and amplitude of the detected signal, or any combination thereof. Each possibility is a separate embodiment.

In some specific embodiments, the data indicative of the neuronal function/activity comprises spatiotemporal propagation including spatial distribution and/or time after stimulation. Each possibility is a separate embodiment.

In some embodiments, the PD comprises non-genetic PD.

According to some embodiments, the PD is selected from one or more of Autism Spectrum Disorders (ASD), Bipolar disorder, Attention Deficit Hyperactivity Disorder (ADHD/ADD), Schizophrenia, Major Depression, Obsessive-Compulsive Disorders (OCD), Rett syndrome, Fragile X Syndrome, Intellectual Developmental Disorder, Down Syndrome, Williams Syndrome, Prader-Willi Syndrome, Angelman Syndrome, Smith-Magenis Syndrome, Epilepsy, Parkinson's disease, and Alzheimer's disease, or any combination thereof. Each possibility is a separate embodiment.

According to a specific embodiment, the PD is Autistic Spectrum Disorder (ASD). According to further specific embodiment, the ASD is non-syndromic idiopathic ASD.

According to another aspect, there is provided method for assessment of a psychiatric disorder (PD), the method comprising:
(a) obtaining a brain organoid; (b) providing one or more stimuli sessions, each session comprising a stimuli provided to the brain organoid; (c) obtaining data recorded in response to the one or more treatment/stimuli sessions, the data is indicative of neuronal function/activity of the brain organoid; (d) determining a brain-organoids behavior based on the recorded data; and (e) applying an AI algorithm on the brain-organoids behavior for classifying the brain organoid based on a degree of similarity of the determined brain-organoid behavior to a predicted behavior of a PD-derived brain organoid and/or a heathy organoid.

According to a specific embodiment, the method comprises an open loop, in which the treatment/stimulus provided to the brain organoid in the one or more sessions are predetermined.

In some embodiments, the AI algorithm is trained on brain-organoids behaviors of a plurality of healthy and/or PD derived brain organoids in response to the predetermined treatment/stimulus, wherein the training data is labeled according to one or more predetermined parameters of the treatment/stimulus. Each possibility is a separate embodiment. (open loop)

In a related embodiment, the AI algorithm is continuously reinforced, based on the determined brain-organoid behavior, to thereby improve the predicted behavior. (open loop)

According to a specific embodiment, the method comprises a closed loop, in which the stimulus provided to the brain organoid is determined according to the determined brain-organoid behavior.

In some embodiments, the AI algorithm is a trained on brain-organoids behaviors of a plurality of healthy and/or PD derived brain organoids, wherein the training data is labeled according to changes in one or more parameters of the treatment/stimulus. (closed loop)

In some embodiments, the method comprises at least two sessions, wherein the stimuli provided in a latter session is determined based on the brain-organoids behavior determined in response to one or more former stimuli sessions. (closed loop)

In some specific embodiments, the stimuli provided in a latter session comprises a positive or negative feedback; and wherein a change in the brain-organoids behavior between a former and the latter sessions is indicative of a learning behavior response of the brain organoid. Each possibility is a separate embodiment. (closed loop)

In some embodiments, classifying the brain organoid is based on a degree of similarity of the learning-behavior response to a predicted learning-behavior response of a PD-derived brain organoid and/or of a heathy organoid. Each possibility is a separate embodiment. (closed loop)

In some embodiments, the method further comprises generating a visual simulation representative of the determined organoid behavior.

In some related embodiments, the visual simulation comprises a computer game configured to evaluate one or more of: memory, cognitive rigidity, motivation, repetitive behavior, attention, social interaction, processing speed, executive function, numerical abilities, and/or facial expression, or any combination thereof. Each possibility is a separate embodiment.

In some embodiments, the method further comprises assessing the severity of PD based on the similarity.

In some embodiments, the method further comprising repeating steps b-d on the brain organoid after treatment thereof with a psychiatric, neurodevelopmental and/or neurological medicament, or any combination thereof. Each possibility is a separate embodiment.

In some embodiments, the method further comprising repeating steps b-d on a brain organoid obtained from a same subject after treatment of said subject with a neurological, neurodevelopmental and/or neurodegenerative medicament, or any combination thereof. Each possibility is a separate embodiment.

In some embodiments, the method further comprising determining an efficacy of the treatment.

In some embodiments, the PD comprises non-genetic PD.

According to some embodiments, the PD comprises one or more neurological, neurodevelopmental and/or neurodegenerative condition, or any combination thereof. Each possibility is a separate embodiment.

In some embodiments the PD is selected from one or more of Autism Spectrum Disorders (ASD), Bipolar disorder, Attention Deficit Hyperactivity Disorder (ADHD/ADD), Schizophrenia, Major Depression, Obsessive-Compulsive Disorders (OCD), Rett syndrome, Fragile X Syndrome, Intellectual Developmental Disorder, Down Syndrome, Williams Syndrome, Prader-Willi Syndrome, Angelman Syndrome, Smith-Magenis Syndrome, Epilepsy, Parkinson's disease, and Alzheimer's disease, or any combination thereof. Each possibility is a separate embodiment.

According to yet another aspect, there is provided a method for training an AI algorithm for determining organoids behavior, the method comprising:
(a) obtaining a plurality of PD-derived brain organoid and a plurality of healthy brain organoids; (b) providing one or more stimuli session(s), each session comprising stimuli provided to the brain organoid; (c) obtaining data recorded in response to the one or more treatment/stimuli session(s), the data is indicative of neuronal function/activity of the brain organoid; (d) labeling the data according to parameters of the one or more stimuli sessions and associating the labeled data with the PD-derived brain organoid and/or with the plurality of healthy brain organoid; (e) applying an AI algorithm on the data to learn patterns and relationships and to adjust parameters of a model for organoid behavior prediction; thereby training the algorithm for determining a brain-organoids behavior based on the data recorded in response to the one or more treatment/stimuli session(s).

According to some embodiments, the AI algorithm is further trained to classify the organoids plurality of PD-derived brain organoids and/or healthy organoids based on the determined organoids' behavior as having ‘PD-derived behavior’ or a ‘heathy behavior’; thereby classifying the brain organoids based on a degree of similarity of their determined behavior to a predicted behavior of a PD-derived brain organoid and/or a heathy organoid. Each possibility is a separate embodiment.

In some embodiments, the obtaining of PD-derived brain organoid comprises organoids having a range of PD severities, and wherein the association of the labeled data with the PD-derived brain organoid comprises associating the labeled data with the range of PD severities; thereby augmenting the prediction behavior model to include a range of severities.

In another embodiment, the data indicative of neuronal function/activity of the brain organoid is divided to a ‘training dataset’ and ‘validation set’, and wherein the ‘validation set’ comprises unlabeled data used to improve model performance.

In some embodiments, the data indicative of the neuronal function/activity comprises spatiotemporal propagation including spatial distribution and time after stimulation, intensity, frequency, and amplitude of the detected signal, or any combination thereof. Each possibility is a separate embodiment.

In a specific embodiment, the data indicative of the neuronal function/activity comprises spatiotemporal propagation including spatial distribution and time after stimulation.

In some embodiments, the AI algorithm is selected from one or more of supervised learning, unsupervised learning, semi-supervised learning, reinforced learning, self-supervised learning, transfer learning, meta-learning, evolutionary algorithms, or any combination thereof. Each possibility is a separate embodiment.

In some embodiments, the AI algorithm is a supervised machine learning algorithm capable of regression and/or classification selected from one or more of Support-vector machines, Linear regression, Logistic regression, Random Forest, Naive Bayes, Linear discriminant analysis, Decision trees, K-nearest neighbor algorithm, Deep Neural networks, Neural networks (Multilayer perceptron), Gradient Boosting Algorithms, Linear Discriminant Analysis, Ridge Regression and Lasso Regression, Elastic Net, Bayesian Regression, Multiclass Classification Algorithms, Similarity learning, or any combination thereof. Each possibility is a separate embodiment.

According to an embodiment, the method of training comprises an open loop training, wherein the treatments/stimuli provided to the brain organoid in the one or more sessions are predetermined.

According to an embodiment, the method of training comprises closed loop training mode, wherein the treatments/stimuli provided to the brain organoid in the one or more sessions is determined according to the determined brain-organoid behavior.

According to some embodiments, the method of training comprises the stimuli provided to the brain organoid comprises one or more of an electrophysiological stimulus, a heat stimulus, a light stimulus, and a drug, or any combination thereof.

In a specific embodiment, the stimuli provided to the brain organoid comprises an electrophysiological stimulus.

According to some embodiments, the brain organoid is derived from one or more of prenatal cells, neonatal cells, cells of a mature baby, cells of a toddler, cells of a child, cells of a teen, and cells of an adult, or any combination thereof. Each possibility is a separate embodiment.

The biological and/or physical/biophysical attribute of the brain organoid, the cells and/or tissue thereof, may be sensed/detected using a microscope/imaging device or a multi-electrode array (MEA) without utilizing any molecular/biochemical or genetic techniques. For example, an external phenotype indicated by a visual optic/light signal may be sensed/detected and visualized directly using a microscope/imaging device, and for example neuronal function/activity provided by electrophysiology and indicated by an electric signal, may be sensed/detected directly using a multi-electrode array (MEA).

Alternatively, the biological and/or physical attribute of the brain organoid, the cells and/or tissue thereof, may be sensed/detected using a microscope/imaging device indirectly utilizing molecular biology/genetic techniques for detection and visualization. For example, the visual optic/light signal comprising the information indicative of the biophysical and/or biological attributes of the brain organoid, the cells and/or tissue thereof, for example, an external phenotype, neuronal function/activity, or genomic abnormalities/structure, may be indirectly detected utilizing molecular biology/genetic/biochemical techniques (e.g., by a genetic reporter for gene expression or Ca⁺² imaging, or Redox imaging, or immunostaining) prior to being sensed/detected and visualized using the microscope/imaging device.

According to some embodiments, the assessment of the one or more signal(s) and/or attribute(s) of the brain organoid comprises assessing biophysical and/or biological attribute indicated by optic/light signal or electric signal; according to some embodiments, the signal and/or attribute comprises biological and/or physical attribute is indicated by a visual optic/light signal or electric signal; according to some embodiments, the optic/light signal or electric signal comprises information indicative of the physical and/or biological attributes of the brain organoid, the cells and/or tissue thereof; according to some embodiments, the electric signal comprises information indicative of spontaneous or induced neuronal function/activity. Each possibility is a different embodiment.

According to some embodiments, the biological and/or physical attribute of the brain organoid, the cells and/or tissue thereof, is sensed/detected using a microscope/imaging device or a multi electrode array (MEA); according to some embodiments, the biological and/or physical attribute of the brain organoid, the cells and/or tissue thereof, is sensed/detected using a microscope/imaging device or a multi electrode array (MEA) without utilizing any molecular/biochemical or genetic techniques; according to some embodiments, an external phenotype, appearance/morphology, 2D/3D structure, shape, size, and texture indicated by a visual optic/light signal is sensed/detected and visualized directly using a microscope/imaging device; according to some embodiments, neuronal function/activity provided by electrophysiology and indicated by an electric signal is sensed/detected directly using a multi-electrode array (MEA). Each possibility is a different embodiment.

According to some embodiments, the biological and/or physical attribute of the brain organoid, the cells and/or tissue thereof, is sensed/detected using a microscope/imaging device indirectly utilizing molecular biology/genetic techniques for detection and visualization; according to some embodiments, the visual optic/light signal comprising the information indicative of an external phenotype, appearance/morphology, 2D/3D structure, shape, size, and texture, neuronal function/activity, gene expression, or genomic abnormalities/structure, is indirectly detected utilizing molecular biology/genetic/biochemical techniques prior to being sensed/detected and visualized using the microscope/imaging device. Each possibility is a different embodiment.

As used herein, the term “biological and/or physical attribute” refers to a characteristic feature of the brain organoid, the cells and/or tissue thereof. The attribute may be indicated by a visual optic/light signal or electric signal. The term “attribute” or “characteristic feature” may be interchangeably used.

According to some embodiments, a biological and/or physical attribute comprises external phenotype, appearance/morphology, 2D/3D structure, shape, size, and texture, spontaneous or induced neuronal function/activity, gene expression, genomic abnormalities/structure, or any combination thereof. Each possibility is a different embodiment.

According to some embodiments, a biological and/or physical attribute may be visualized directly using a microscope/imaging device, the group consists of external phenotype, appearance/morphology, 2D/3D structure, shape, size, and texture, or any combination thereof; according to some embodiments, a biological and/or physical attribute consisting of spontaneous or induced neuronal function/activity provided by electrophysiology. Each possibility is a different embodiment.

According to some embodiments, a biological and/or physical attribute may be indirectly detected utilizing molecular biology/genetic/biochemical techniques prior to being sensed/detected and visualized using the microscope/imaging device, and comprises external phenotype, appearance/morphology, 2D/3D structure, shape, size, and texture, spontaneous or induced neuronal function/activity provided by a genetic reporter for Ca⁺² imaging, or Redox imaging, gene expression provided by immunostaining or genetic reporter, genomic abnormalities/structure provided by genomic/genetic examination, or any combination thereof. Each possibility is a different embodiment.

According to some embodiments, a biological and/or physical attribute may be detected utilizing molecular biology/genetic/biochemical techniques without utilizing any sensor such as an imaging device or electrodes. For example, a biological and/or physical attribute may be detected using analysis of gene expression or genetic analysis provided by next-generation sequencing (NGS) or any other computational data/bioinformatics approaches that relies on molecular biology/genetic/biochemical methods.

According to some embodiments, an assessment of a biological and/or physical attribute comprises utilizing brain organoids derived from primary PD-cells obtained from urine excretion collected from a subject suffering from PD.

According to some embodiments, a change in at least one biological and/or physical attribute of a PD-derived brain organoid, the cells and/or tissue thereof comprises a signal and/or attribute directly visible using a light microscope.

According to some embodiments, a change in at least one biological and/or physical attribute of a PD-derived brain organoid, the cells and/or tissue thereof comprises the brain organoid and 3D tissue thereof size.

According to some embodiments, a change in at least one biological and/or physical attribute of a PD-derived brain organoid, the cells and/or tissue thereof comprises neural projections length/size of the brain organoid and cells thereof, relative to the brain organoid size.

As used herein the terms “size” and “length” may refer to the neural projections of the brain organoid and may be interchangeably used.

According to some embodiments, a change in at least one biological and/or physical attribute comprising the brain organoid size, and/or length of neural projections thereof, clearly distinguishes a PD-derived brain organoid from a healthy brain organoid.

According to some exemplified embodiments, at the indicated timeframe of up to about 17 days in culture a PD-derived brain organoid may be at least about 20 μm greater in size, at least about 30 μm greater in size, at least about 40 μm greater in size, preferably at least about 50 μm greater in size, preferably at least about 60 μm greater in size, more preferably at least about 70 μm greater in size, more preferably at least about 80 μm greater in size, even more preferably at least about 90 μm greater in size, even more preferably at least about 100 μm greater in size, even more preferably at least about 110 μm greater in size, even more preferably at least about 120 μm greater in size, than a healthy brain organoid. Each possibility is a separate embodiment.

According to some embodiments, at a timeframe longer than 3 weeks in culture a PD-derived brain organoid may be at least about 100 μm greater in size, at least about 200 μm greater in size, at least about 300 μm greater in size, at least about 400 μm greater in size, at least about 500 μm greater in size, than a healthy brain organoid. Each possibility is a separate embodiment.

According to some exemplified embodiments, at the indicated timeframe of up to about 17 days in culture a PD-derived brain organoid may be between about 20 μm to 200 μm greater in size, preferably between about 40 μm to 180 μm greater in size, even more preferably between about 50 μm to 150 μm greater in size, even more preferably between about 50 μm to 120 μm greater in size. Each possibility is a separate embodiment.

According to some embodiments, at a timeframe longer than 3 weeks in culture a PD-derived brain organoid may be between about 20 μm to 500 μm greater in size, between about 20 μm to 400 μm greater in size, between about 20 μm to 300 μm greater in size. Each possibility is a separate embodiment.

According to some exemplified embodiments, at the indicated timeframe of up to about 17 days in culture a PD-derived brain organoid may have at least about 10% greater ratio of “organoid to outward neural projections size” (μm), at least about 15% greater ratio, preferably at least about 20% greater ratio, preferably at least about 25% greater ratio, preferably at least about 30% greater ratio, even more preferably at least about 35% greater ratio, even more preferably at least about 40% greater ratio, even more preferably at least about 45% greater ratio, even more preferably at least about 50% greater ratio, than that of a brain organoid derived from healthy cells. Each possibility is a separate embodiment.

According to some embodiments, at a timeframe longer than 3 weeks in culture a PD-derived brain organoid may have at least about 75% greater ratio, least about 100% greater ratio, least about 125% greater ratio, least about 150% greater ratio, least about 175% greater ratio, least about 200% greater ratio, least about 250% greater ratio, of “organoid to outward neural projections size” (μm). Each possibility is a separate embodiment.

According to some exemplified embodiments, at the indicated timeframe of up to about 17 days in culture a PD-derived brain organoid may have a ratio of “organoid to outward neural projections size” (μm), between about 10% to 100% greater, preferably between about 20% to 80% greater, even more preferably between about 25% to 60% greater, than that of a brain organoid derived from healthy cells. Each possibility is a separate embodiment.

According to some embodiments, at a timeframe longer than 3 weeks in culture a PD-derived brain organoid may have a ratio of “organoid to outward neural projections size” (μm), between about 10% to 250% greater, between about 10% to 200% greater, between about 10% to 100% greater, than that of a brain organoid derived from healthy cells. Each possibility is a separate embodiment.

As used herein, the term “pre-treatment” refers to a treatment applied to a brain organoid, or a cell obtained, transformed, or differentiated to generate the brain organoid, prior to assessment of a change/difference in one or more signal(s) and/or attribute(s) of the brain organoid, and/or tissue and/or cells thereof (i.e., assessment of PD).

A pre-treatment may comprise inhibition or activation of a specific gene expression, expression of a genetic reporter, or exposure to electric pulse, exposure to optic/light signal, exposure to heat, exposure to a chemical agent/drug, culturing conditions that include starvation/nutrient deprivation and/or hypoxia, or any combination thereof.

According to some embodiments, pre-treatment comprises one or more of inhibition or activation of a specific gene expression, expression of a genetic reporter, or exposure to electric pulse, exposure to optic/light signal, exposure to heat, exposure to a chemical agent/drug, culturing conditions that include starvation/nutrient deprivation and/or hypoxia, or any combination thereof. Each possibility is a different embodiment.

As used herein, the term “learning-behavior response” refers to a learning process that results from or reinforced by, repeatedly pairing a feedback stimulus with a preceding neural behavior and may be computed as a change in neuronal function/activity (i.e., behavior/simulated behavior) of a brain organoid (change in computational simulation of information indicative of the neuronal function/activity) in response to a positive or negative feedback treatment (i.e., feedback stimulus). The learning-behavior response is a computational functional analysis. The terms “learning-behavior response” and “reinforcement learning” may be interchangeably used.

According to some embodiments, the assessment of PD further comprises assessing a learning-behavior response of the prenatal or neonatal derived-brain organoid, compared to a pre-determined learning-behavior response of a PD-derived brain organoid and/or a healthy brain organoid.

According to some embodiments, the assessment of a learning-behavior response of the prenatal or neonatal derived-brain organoid comprises (a) obtaining input data comprising information indicative of neuronal function/activity of the prenatal or neonatal derived-brain organoid; (b) generating a computer simulation of the prenatal or neonatal derived-brain organoid behavior; (c) determining a positive or negative feedback treatment based on the simulated behavior of the prenatal or neonatal derived-brain organoid; (d) delivering the determined positive or negative feedback treatment to the prenatal or neonatal derived-brain organoid using a stimuli/manipulation system; (e) obtaining input data from the prenatal or neonatal derived-brain organoid comprising information indicative of neuronal function/activity in response to the positive or negative feedback treatment delivered; (f) simulating the behavior of the prenatal or neonatal-derived brain organoid in response to the positive or negative feedback treatment delivered; (g) repeating steps c to f X times; and (h) compute an output score indicative of the learning-behavior response based on a change in the behavior of the prenatal or neonatal derived-brain organoid compared to a pre-determined learning-behavior response of a PD-derived brain organoid.

In some embodiments, X is an integer between 1-10000, 1-1000, or 1-100. Each possibility is a separate embodiment.

According to an aspect of the disclosure, there is provided a system for prenatal and/or neonatal assessment of psychiatric disorder (PD), the system comprising (i)
a prenatal or neonatal-derived brain organoid and a PD-derived brain organoid; (ii) a sensor coupled to a recorder capable of detecting and archiving one or more signal(s) and/or attribute(s) of at least the prenatal or neonatal-derived brain organoid; and (iii) at least one micro-controller unit (MCU) configured to integrate and/or transmit information derived from the one or more signal(s) and/or attribute(s); (iv) a computer/processor configured to (a) obtain input data from a plurality of prenatal or neonatal-derived brain organoid and from a plurality of PD-derived brain organoid, wherein the input data comprises a plurality of information derived from the one or more signal(s) and/or attribute(s); and (b) apply an AI algorithm to determine a probability or range of probability of the prenatal-derived brain organoid being derived from PD cells/subject.

Advantageously, the herein-disclosed system provides a prenatal or neonatal assessment of PD based on an assessment of a change/difference in one or more signal(s) and/or attribute(s) of the brain organoid, and/or tissue and/or cells thereof.

As used herein, the term “imaging device” refers to any light-sensitive device capable of producing a visual image and may include or be coupled to a camera.

As used herein, the term “sensor” refers to a light-sensitive or a voltage-sensitive device that produces current when absorbs photons or electrons. For example, a sensor may be an imaging device, or a multi-electrode array (MEA).

According to some embodiments, the sensor coupled to a recorder capable of detecting and archiving comprises an imaging device coupled to a camera; according to some embodiments, the sensor coupled to a recorder capable of detecting and archiving comprises a multi-electrode array (MEA) coupled to one or more recording head stage (RHS); according to some embodiments, the at least one signal and/or attribute detected and archived by the imaging device coupled to a camera is an optic/light signal; according to some embodiments, the at least one signal and/or attribute detected and archived by the multi-electrode array (MEA) is an electric.

According to some embodiments, the sensor is capable of detecting optic/light signal or electric signal comprising information indicative of physical and/or biological attributes of the brain organoid, the cells and/or tissue thereof. Each possibility is a different embodiment.

According to some embodiments, at least one signal and/or attribute detected and archived is an optic/light signal, wherein the optic/light signal comprises information indicative of physical and/or biological attributes of the prenatal or neonatal-derived brain organoid; according to some embodiments, the physical and/or biological attribute indicated by the optic/light signal comprises external phenotype, appearance/morphology, 2D/3D structure, shape, size, and texture, or any combination thereof; according to some embodiments, the physical and/or biological attribute indicated by the optic/light signal further comprises gene expression provided by immunostaining or genetic reporter, and genomic abnormalities/structure provided by genetic examination, or any combination thereof; Each possibility is a different embodiment.

According to some embodiments, at least one signal and/or attribute detected and archived is an electric signal or an optic/light signal. According to some embodiments, the electric signal or optic/light signal comprises information indicative of spontaneous or induced neuronal function/activity of the prenatal-derived brain organoid. According to some embodiments, the optic/light signal comprises information indicative of neuronal function/activity is omitted from a genetic reporter.

As used herein, the term “micro-controller unit (MCU)” refers to a processor.

According to some embodiments, the information indicative of physical and/or biological attributes of the prenatal or neonatal-derived brain organoid is transferred from the sensor coupled to a recorder to the MCU; according to some embodiments, the information indicative of the neuronal function/activity is transferred from the sensor coupled to a recorder to one or more of same or different MCU; according to some embodiments, the MCU is connected to a wireless radio transmitter (RF) or a micro transmitter (MT) connecting it to at least one remote MCU; according to some embodiments, the MCU is connected to the processor/computer. Each possibility is a different embodiment.

According to some embodiments, the system further comprises a source of stimuli/manipulation capable of delivering treatment to the prenatal or neonatal-derived brain organoid.

According to some embodiments, the treatment delivered by the source of stimuli/manipulation to the prenatal or neonatal-derived brain organoid is one or more of electric pulse, optic/light stimulus, heat, a chemical agent/drug, or any combination thereof; according to some embodiments, information indicative of the treatment delivered by the source of stimuli/manipulation to the prenatal brain organoid in culture comprises information about concentration, temperature, duration, intensity, frequency and/or amplitude of the stimuli, or any combination thereof; according to some embodiments, information indicative of the neuronal function/activity comprises duration, intensity, frequency and/or amplitude of the detected signal or any combination thereof; according to some embodiments, information indicative of the neuronal function/activity further comprises spatial information. Each possibility is a different embodiment.

The system may be used for the purpose of drug screening and/or evaluation of treatment with a drug applied directly in culture. Non-limiting examples of drugs that may be applied directly in culture include psychiatric/neurologic drugs commonly used with PD patients in the clinic. According to some embodiments, the system is used for drug screening and/or evaluation of treatment with a drug applied directly in culture on PD-derived brain organoid; according to some embodiments, the system is used for drug screening and/or evaluation of treatment with a drug applied directly in culture on prenatal or neonatal-derived brain organoid or on healthy organoid. Each possibility is a separate embodiment; according to some embodiments, the drug for screening and/or evaluation is a known psychiatric/neurologic drug in medical use, or a potential drug for the treatment of PD.

Non-limiting examples of psychiatric/neurologic drugs comprise selective serotonin reuptake inhibitors (SSRIs), Selective serotonin and norepinephrine inhibitors (SNRIs), beta-blockers, stimulants, serotonergic drugs, tricyclic antidepressants, atypical antipsychotic agents, lithium, alpha-2 agonists.

According to another aspect of the disclosure, there is provided a brain organoid for prenatal and/or neonatal assessment of psychiatric disorder (PD).

Advantageously, the herein-disclosed brain organoid provides a prenatal or neonatal assessment of PD based on an assessment of a change/difference in one or more signal(s) and/or attribute(s) of the brain organoid, and/or tissue and/or cells thereof.

According to some embodiments, the brain organoid for prenatal assessment of psychiatric disorder (PD) in human prenatal and/or neonatal cells, is generated from the human prenatal or neonatal cells.

According to some embodiments, the PD comprises one or more of Autism Spectrum Disorders (ASD), Bipolar disorder, Attention Deficit Hyperactivity Disorder (ADHD/ADD), Schizophrenia, Depression, Obsessive-Compulsive Disorders (OCD), Epilepsy, or any combination thereof.

According to some embodiments, the prenatal or neonatal-derived brain organoid, comprises at least a cerebral and/or cortical tissue/area/region.

According to some embodiments, the prenatal or neonatal-derived brain organoid comprises cells treated to alter gene expression, to express a genetic reporter or comprises cells exposed to one or more stimuli/manipulation comprising electric pulse, optic/light signal, heat, a chemical agent/drug, or any combination thereof. Each possibility is a separate embodiment.

The following examples are presented in order to illustrate some embodiments of the invention more fully. They should in no way be construed, however, as limiting the broad scope of the invention. One skilled in the art can readily devise many variations and modifications of the principles disclosed herein without departing from the scope of the invention.

### EXAMPLES

### Materials and Method

Protocol for preparation of cortical organoids from hiPSCs-Cortical organoids were prepared based on Rosebrock N. et al., Nature Cell Biology 24, 981-995 (2022). Briefly, on day 0, hiPSC colonies were first incubated with 1 ml accutase up to 10 min until colonies detached. The colonies were then triturated until single cells were obtained. The accutase enzyme was neutralized by washing with hESC/KSR medium and centrifugation at 270 g for 5 min. Single cells were resuspended in 1 ml hESC/KSR medium containing FGF2 (4 ng/ml) and ROCK inhibitor (50 μM). The cells were enumerated and the volume of the hESC/KSR medium was adjusted along with FGF2 and ROCK inhibitor to a concentration of 9,000 cells per 150 μl. Suspended single cells were plated on a 96-well U-bottom low-attachment plate. The plate was inspected for cell aggregation and formation of embryoid bodies (EBs) on day 1. On day 2, half of the medium was aspirated without disturbing aggregates and 150 μl hESC/KSR medium was added to a total of 225 μl hESC medium along with the appropriate inhibitor molecule-SB-431542 (10 μM), LDN (200 nM) or XAV-939 (3.3 μM)—or a combination thereof. FGF2 and ROCK inhibitor were withdrawn once the EBs reached a size of approximately 350 μm. On day 4, 150 μl medium was removed and replaced with fresh 150 μl hESC/KSR medium along with the corresponding inhibitor molecules. On day 6, the organoids were transferred into a low-attachment 24-well plate along with N2 neural induction medium. Every alternate day, medium was aspirated and replaced by an equal volume of fresh N2 medium along with factors until day 11. On day 11, the organoids were embedded in 30 μl Matrigel droplets and incubated for 30 min in the incubator, after which they were transferred into a six-well low-attachment plate containing N2/NB medium along with 1% B27 without RA. On day 13, a medium change was made using the same medium from day 11. On day 15, the entire supernatant medium was removed and replaced with fresh medium containing N2/NB medium along with 1% B27 with RA; the organoid dishes were transferred onto an orbital shaker and the medium was changed daily. For long-term organoid culture, Matrigel (1%) was added directly to the culture medium and the medium was changed every 2 d.

### Example 1—Structural Characterization of Cortical Brain Organoids Generated from ASD Cells

Developmental and morphological differences in 3D structure between Autistic Spectrum Disorder (ASD)-derived cortical brain organoids and healthy-derived brain organoids were examined.

Cortical brain organoids were generated from primary epithelial cells of a teen subject (i.e., about 13-19 years) who suffers from non-genetic idiopathic non-syndromic Autistic Spectrum Disorder (ASD), or from primary epithelial cells of a teen donor considered as healthy with respect to psychiatric disease (PD).

Primary epithelial cells were isolated from urine excretion samples collected from the subjects. These cells were obtained from both healthy and ASD individuals are subsequently reprogrammed into their respective iPSCs, namely healthy iPSCs and ASD-iPSCs. These iPSCs were then expanded in culture and subjected to cortical organoids differentiation process, resulting in the generation of both healthy and ASD cortical organoids.

Reference is made to the protocol for preparation of cortical organoids from hiPSCs.

As can be seen in FIGS. 2A-2B a population of healthy and ASD-derived cortical brain organoids were generated and grow. The main developmental stages-primary cells, iPSCs, and organoids were compared between healthy, and ASD-derived cortical including at day 6 and day 17 (FIG. 2A). The generated 3D brain organoids were visible to the naked eye (FIG. 2B).

As can be seen in FIG. 2A, at least two surprising morphological/structural differences were found to exist between the ASD-derived cortical brain organoids and the healthy-derived brain organoids, just by visualizing the organoids using light microscope.

Further surprising was the second difference/attribute in the size of the neural projections of the organoids facing outwards of organoid.

Advantageously, the differences in organoids' size, including a difference in its neural projections size, were unambiguous and appear to distinguish an ASD-derived brain organoid from a healthy brain organoid just by visualizing them in a simple illumination contrast imaging device.

These Size differences were quantified, as shown in FIGS. 2C-2D, that also illustrate the way the measurement was performed (in a small square to the right of the chart). The quantification of those difference exhibited very high statistically significant differences between the healthy and ASD-derived organoids.

First, differences in organoids' size were evaluated by measuring diameter (A) (μm). ASD-derived organoids exhibited a mean diameter (A) (μm) of about 660+/−25 μm, whereas healthy control brains exhibited a mean diameter of 550+/−25 μm (FIG. 2C). Size differences were measured at day 6 and are apparent from about day 4 and at least up to about 5 months after, whereas the timeline is in accordance with the protocol preparation of cortical organoids from hiPSCs.

In addition, to describe the size difference in its neural projections, the ratio between the organoid size (A) and its outwards neural projection size (B) (μm), was measured, and was calculated as ((A+B)/A) (μm). The result is presented as three individual independent repeats in (FIG. 2D), indicative of about 1.5 fold change at the ratio values, wherein ASD-derived organoids having a ratio of about 1.45 (μm)+/−0.1 μm, and healthy control brains having a ratio of about 1.1 (μM)+/−0.1 μm. Differences in the ratio between the organoid size (A) and its outwards neural projection size (B) (μm) were measured at day 17 and are apparent from about day 10 and at least up to about 2 months after, whereas the timeline is in accordance with the protocol preparation of cortical organoids from hiPSCs.

To verify these results, the organoid size/diameter (A) at day 6, and the ratio between the organoid size/diameter (A), and its outwards neural projection at day 17 ((A+B)/A) (μm), were evaluated again in ASD-derived organoids and healthy control organoids cultured under normal conditions, as well as in the presence of an inhibitor or an activator of the WNT signaling pathway CHIR99021 (FIGS. 2E-2H).

As can be seen in FIG. 2E and quantified and illustrated in FIG. 2F the mean organoid diameter (A) of ASD-derived organoids at day 6 under normal culturing conditions, was about 500 μm while the mean diameter (A) of the control organoids was about 360 μm, indicating a clear, statistically significant difference of about 140 μm that affirms the previous observation presented in FIG. 2A and quantified in FIG. 2C.

As indicated in FIG. 2F the presence of inhibitor did not change the difference in the mean organoid diameter (A) of ASD-derived and healthy organoids (compare ‘inhibitor’ with ‘normal’), whereas the presence of the activator caused an elevation in the mean organoid diameter (A) of both the ASD-derived and the healthy organoids by about 20% (compare ‘activator’ with ‘normal’), overall increasing the observed difference between the two from about 140 μm to about 200 μm (compare for the activator: ‘ASD’ with ‘healthy’). Incubation of the organoid with the activator increased the size differences between the ASD-derived and the healthy organoids by about 60 μm, with respect to organoids grow under normal conditions i.e., w/o the activator.

As can be seen in FIG. 2G and quantified and illustrated in FIG. 2H, the mean ratio between the organoid size/diameter and its outwards neural projection ((A+B)/A) (μm) of ASD-derived organoids at day 17 under normal culturing conditions, was about 1.5 μm while the mean ratio of control organoids was about 1.0 μm, indicating a clear, statistically significant difference of about 50% that affirms the previous observation presented in FIG. 2A and quantified in FIG. 2D.

As indicated in FIG. 2H the presence of inhibitor did not change the difference in the mean ratio between the organoid size/diameter and its outwards neural projection ((A+B)/A) (μm) of ASD-derived and healthy organoids (compare ‘inhibitor’ with ‘normal’), whereas the presence of the activator slightly reduced the measured value of ASD-derived organoids and slightly elevated the value of healthy organoids (compare ‘activator’ with ‘normal’), overall decreasing the observed difference between the two (compare for the activator: ‘ASD’ with ‘healthy’).

In conclusion, ASD-derived brain organoid may exhibit a size difference of between about 25 μm to 140 μm greater size/diameter (A) relative to brain organoid derived from healthy cell; and may also have between about 15% and 60% greater ratio of organoid to outward neural projections size ((A+B)/A), compared with a brain organoid derived from healthy cells.

Further advantageous and surprising, is the disclosed use of an activator to further increase/widen the difference in mean size/diameter (A) of ASD-derived relative to healthy organoids (up to 200 μm), making the objective of distinguishing between the two utilizing simple means for visualization, such as any illumination contrast method, a light microscope or the like, even more straight forward than it is under normal conditions.

Notwithstanding, the above-described differences is size and shape of the organoid, it was also found, for example in Shank3 ASD-derived organoids, that ASD-derived organoids may deviate from the characteristic size and shape of healthy organoids, by exhibiting a significantly smaller size of organoid and less sprouting of its projections.

Cortical brain organoids were generated from primary cells of subjects (i.e., about 10-15 years) who suffers from genetic (Shank3) and non-genetic idiopathic non-syndromic Autistic Spectrum Disorder (ASD), or from cells of a teen donor considered as healthy with respect to psychiatric disease (PD).

As can be seen in FIGS. 2I-2J, organoids derived from shank3 autistic subjects (ASD-Shank3), and evaluated at day 10 according to the timeline described in the protocol for preparation of cortical organoids, deviate from healthy organoids by having smaller size of about 85 μm in diameter (A) (deviation is between about 25 μm and about 120 μm) (FIG. 2I). Furthermore, shank3-ASD organoids deviated from healthy organoids also by having reduced or enhanced amount/number of projections (over or under-expression), as evaluated here at day 18 (FIG. 2J).

In summary, advantageously and surprisingly, these results show that with respect to healthy organoid, an ASD-derived brain organoid exhibit differences in shape indicative by enhanced or reduced sprouting of its neural projection, and further that an ASD-derived brain organoid exhibit increased or decreased size.

Therefore, it is concluded that ASD-derived brain organoids deviate from healthy organoids by their size and shape, and accordingly, may be differentiated from them based on being larger or smaller, and having more or less sprouting, just by visualizing the organoid and its neural projection.

### Example 2—Characterization of Dendritic Structure of Cortical Brain Organoids Generated from ASD Cells—Dendritic Spines

Next, the inventors further explored morphological differences related to 3D structures of the organoid between Autistic Spectrum Disorder (ASD)-derived cortical brain organoids and healthy-derived brain organoids.

Dendritic spines serve as a storage site for synaptic strength and help transmit electrical signals to the neuron's cell body.

To investigate whether morphological and structural characteristic of dendrites, including their dendritic spines may be used to differentiate healthy from ASD-derived organoid, cortical organoids were generated, and their neuronal dendrites were directly visualized using Adeno Associate Virus (AAV) expressing green fluorescence marker.

Cortical organoids were generated from epithelial cells isolated from same urine samples of heathy and ASD teen subject described in Example 1, and generated according to same protocol. 6-month-old 3D organoids were then enzymatically digested using Papain, and dissociated into 2D neuronal culture where they were infected at day 13 with AAV1-GAC-GreenLantern virus (1/10 dilution) and maintained another 7 days before being visualized (a total of 20 days in 2D culture). Timeline is in accordance with the protocol preparation of cortical organoids from hiPSCs.

As can be seen in FIGS. 3A-3C, fluorescence images of dendrites of 6-month-old organoids were acquired using a fluorescence microscope, showing dendritic spines.

Comparative image analysis of dendritic projections of healthy (FIG. 3A) and ASD-derived (FIG. 3B) brains organoids immediately pointed towards an additional advantageous and surprising finding, suggesting that dendritic spines along the projections of ASD-derived organoids are readily identifiable, and distinguishable from healthy projections, partially due to increased density of spines along the projection (i.e., number of spines per projection), generally suggesting increased activity of the spines/synapse.

FIG. 3C present another representative image emphasizing this surprising finding of increased number of spines along the projections of ASD-derived organoid (arrows pointing on spines).

Advantageously, because the increased number of spines was clearly visible relative to projection of healthy organoid, the whole shape and structure of the dendritic projection appears different, i.e., looks more grainy just by visual appearance, and even without determining the absolute number of spines.

The images show ASD-derived organoid spines occupies the projections at higher numbers and having larger size/volume, relative to healthy projections. In accordance, these synaptic structures exhibited larger variability in spine shape and volume.

To conclude, it was found that dendritic projections of ASD-derived brains organoids are characterized by remarkable morphological/structural variations compared to healthy organoids' projections, attributed to their spine density, shape and/or volume, but especially due to their density along the projections, and that these may be used to differentiate healthy from ASD-derived organoid and possibly to assess severity of PD, through quantification assay.

### Example 3—Molecular Characterization of Cortical Brain Organoids Generated from Prenatal Cells

Advantageously, cortical brain organoids were generated from human primary prenatal cells, and characterized in-situ using immunostaining for spatial expression of specific developmental markers.

In addition, a prenatal derived iPSC line was established to provide convenient and consistent resource for prenatal-derived healthy organoids.

Primary neonatal cells—Human Amniotic Epithelial Cells (HAEpiC)—were isolated from an amniotic fluid sample collected from a fetus by amniotic fluid test (Amniocentesis), and were used to generate iPSC line followed by generation of 3D self-assembled structures.

Developmental differences, including in morphology/structure and in gene expression, between the primary neonatal cells, the iPSC, and the 3D organoids derived therefrom, were examined.

As seen in FIG. 4A the main stages of generating cortical brain organoids from prenatal cells, are identical to those used to generate ASD-derived organoids as presented in Example 1, FIG. 2A, performed essentially according to the same protocol for preparing organoids, and include generation of iPSC from the primary neonatal cells followed by development and growth of 3D at least partially self-assembled structures, i.e., cortical organoids.

Exemplified herein are human amniotic epithelial cells (HAEpiCs) isolated from amniotic fluid sample collected from a fetus by amniotic fluid test (Amniocentesis). The obtained cells were reprogrammed into induced pluripotent stem cells (iPSCs) and a line derived from the HAEpiCs (HAEpiC-iPSC line) was established, to provide a source based on which populations of 3D cortical organoids, visible to the naked eye, were generated and grown at least for 130 days (FIG. 4B). timeline is according to the same protocol for preparing organoids.

To characterize the organoid for spatial expression of specific developmental markers, whole mount immunostaining was performed to cells of the HAEpiC-iPSC line, as well as for the 3D generated organoids (FIGS. 4C-4E).

First, as seen in FIG. 4C, immunostaining of cells of the HAEpiC-iPSC line showed that the cells express pluripotent markers including SOX2, NANOG, and OCT3/4, suggesting these cells are undifferentiated and have the capacity for self-renewal.

However, during organogenesis, altered expression of these transcription factors influences the stem cells to lose their pluripotency and turn toward a lineage selection.

To assess lineage selection of HAEpiC-cortical organoids, the 3D brains were subjected to whole mount immunostaining for spatial expression analysis of neuronal marker (TUJ1) and neural stem cell markers (SOX2), at day 42, and at day 130.

As can be seen in FIG. 4D, neural vesicle/rosette structures were observed at 42 days old organoids (top; I.). The enlarged image of the neural vesicle/rosette display neural stem cells (SOX2) around the ventricle and neurons (TUJ1) surrounding the neural stem cells (bottom; II.).

In addition, as can be seen in FIG. 4E expression of TUJ1 and SOX2 was detected in 130 days old HAEpiC-Cortical Organoids, thereby demonstrating the presence of neurons.

In summary, cortical brain organoids were self-assembled into an organized 3D structure having neural vesicle/rosette motifs/structures including both differentiated neurons and neural stem cells arranged in a coordinated manner. The organoids were generated from primary amniotic prenatal cells, and a prenatal derived iPSC line was established therefrom.

Advantageously, it is herein exemplified that cortical neurons can be generated from prenatal or neonatal cells.

### Example 4—Electrical Activity Recording from Prenatal Derived Brain Organoid

Prenatal and/or neonatal assessment of psychiatric disorder (PD) includes assessing one or more signal(s) and/or attribute(s) of a prenatal and/or neonatal brain organoid. In addition to the hereinabove structural attributes disclosed in previous examples, and including organoid structure as exemplified in Example 1 and dendritic structure as exemplified in Example 2, it is disclosed that prenatal and/or neonatal assessment of PD may be performed by assessing one or more electrical activity signal including signal detected by electrophysiological recording or genetic reporter imaging.

The electrical activity is detected, and the data recorded is indicative of neuronal function/activity of the prenatal or neonatal brain organoid. The electrical activity is recorded in response to one or more stimuli sessions, each of the one or more stimuli sessions includes providing a stimuli/treatment to the brain organoid. Next, a brain-organoid behavior is determined based on the response of the neural network.

In other words, a brain-organoid behavior is determined based on electrical activity recordings from prenatal or neonatal brain organoid that was provided with stimuli (including a predetermined stimuli (open mode) or a feedback stimuli (closed mode) during each of one or more stimuli sessions provided to the organoid.

Next, the prenatal or neonatal brain organoid is classified based on a degree of similarity of the determined organoid behavior to a predicted behavior of PD-derived brain organoids and/or heathy organoids; wherein the classification is performed by an AI algorithm trained on a plurality of PD-derived brain and/or a heathy organoid; and wherein the PD-derived brain organoids includes a range of severity; thereby facilitating prenatal and/or neonatal assessment of the “severity of PD”.

In accordance with the above, and in order to show that signal, namely electrical activity, can be detected and recorded from prenatal and/or neonatal derived-cortical organoids, Human Amniotic Epithelial Cells (HAEpiC)-derived 3D cortical organoids were generated from the established iPSC line derived from the HAEpiCs (HAEpiC-iPSC line) and allow to grow in culture for 6 months, before subjecting them to enzymatic digestion and processing thereof to dissected neuron in 2D culture (or for example, processing thereof to 3D clamps, small pieces of organoid using mechanical means).

The resultant 2D neuronal culture was then subjected to electrical activity measurements utilizing genetic reporter imaging (FIGS. 5A-5B) and electrophysiological recording (FIGS. 5C-5D).

As can be seen in FIG. 5A, for detection of spontaneous electrical activity from the 2D neuronal culture derived from the HAEpiC-derived 3D cortical organoids, the neurons were transduced to express genetically encoded calcium indicator (GCaMP), and fluorescence imaging of calcium influx was performed.

As can be seen in FIG. 5B, changes in fluorescence intensity in individual neuron were observed/recorded and the cells were marked as ROIs (yellow circle) for further quantification of the signal indicative of calcium oscillations in the representative cells. Seen are examples traces from 6 arbitrary ROI indicating change in fluorescence (ΔF/F) over time (normalized to 1).

As can be seen in FIG. 5C, for detection of spontaneous electrical activity from the 2D neuronal culture derived from the HAEpiC-derived 3D cortical organoids, the dissected neurons were cultured on a 64 electrode Multi Electrode Analysis (MEA) plate (I.) and 2 min of spontaneous signal/activity were recorded from a single electrode of a MEA (II.). The figure also shows a 20-sec segment from the 2 min recording (expanded from white dashed box; III.), as well as an overlay of all detected spikes (IV.).

Nevertheless, signal of spontaneous activity is not a meaningful signal that is indicative of true neuronal functioning that codes information. Therefore, in order to be able to distinguish between a network response related to PD or to healthy, one or more stimuli sessions are provided to the 3D organoid or the 2D neuronal culture, so an organoid behavior can be determined (whether PD like behavior or healthy like behavior) based on the response to the provided stimuli.

The one or more stimuli session are provided using a stimuli system.

A non-limiting example for a setup/system that can be used for electrophysiological measurements from prenatal and/or prenatal organoids in order to assess severity of PD, includes an ‘organ-on-a chip’ ‘all-in-one’ custom device, designed to include at least: a brain organoid in 2D/3D culture and a sensor (multi-array electrode (MAE)) coupled to a recorder (recording head stage (RHS)) (FIG. 5D; I.). The device may further include a micro-controller unit (MCU) and a processor, or as exemplified herein it may be connected to MCU, and to a stimuli/manipulation system capable of providing the brain organoid with one or more stimuli session (may be the same unit as seen in FIG. 5D; II.). The computer configured to determining an organoid behavior based on the recorded data of neuronal activity in response to the provided stimuli (FIG. 5D; III.), and further classify it using AI algorithms.

FIG. 5E shows an enlarged picture of the custom device of FIG. 5D (I).

The device is presented in detail in FIGS. 7C-7D.

FIG. 5F further exemplifies the interface between the brain organoid and the multi electrode array (MEA) belonging to the customized device. Here the organoid is an ASD-derived cortical brain organoid (day 90) plated.

As can be seen in FIG. 5F (left; ON; red dots) a sub-set of electrodes including 3 out of 59 ‘channels’ were then activated to send electrophysiological stimuli in a spatiotemporal controlled manner including a desired pattern and parameters.

A similar procedure was performed with 2D neuronal culture of the ASD-derived organoid. The interface between the cells of the dissociated brain organoid and the multi electrode array (MEA) belonging to the customized device, can be seen in FIG. 5G.

The ASD-derived cells in the culture were dissociated from a brain organoid after its formation (126 days). The 3D brain organoid was processed/enzymatically digested to dissociated cells, which were plated in 2D culture and positioned in the plate holder of the customized made device.

The system architecture and basic design principles are further illustrated in detail in FIGS. 7A-7B, and exemplified in following Examples 5-6 including exemplifying an assessment of signals of electric activity/function in response to stimuli sessions (open and closed), and further determination of PD behavior or healthy behaviors represented by computer simulations assaying cognitive function (FIGS. 6A-6C). Finally, a use of the system for personalized assessment of treatment efficacy with a medicament is also described (Example 7).

To summarize the current Example, advantageously, it was herein demonstrated that electrical activity signal can be detected and recorded from prenatal and/or neonatal derived-cortical organoids in 2D neuronal culture and 3D organoids, the electrical activity signal includes electrophysiological recording and/or genetic reporter imaging.

### Example 5—Assessment of a Behavior Response Associated with a PD-Like Brain Organoid Using Open Loop Approach

A signal that is detected by the system provided herein, and results from spontaneous activity is not a meaningful signal that is indicative of true neuronal functioning that codes information. Therefore, in order to be able to distinguish between a network response related to PD or to healthy, one or more stimuli sessions are provided to the 3D organoid or the 2D neuronal culture, so an organoid behavior can be determined (whether PD like behavior or healthy like behavior) based on the response to the provided stimuli.

The following exemplifies the steps of the method for assessing PD related to the analyses of the recorded data, and determining the organoid behavior based on a simulation of the stimuli-response sessions that are visualized as a computer game, and further classifying the behavior (whether PD like behavior or healthy like behavior) or scoring the result as a likelihood for certain severity of PD.

The organoids of the example were generated and stimulated, and their network response was recorded.

The visual simulation presented in FIG. 6A exemplifies organoid behavior that was determined by the provided system based on the network response.

The visual simulation presented in FIG. 6B is a theoretical example of how the behavior of the 2D neuronal culture dissected from the organoid can be visualized after determining it based on the network response.

A non-limiting example of a type of analysis that underly determining the network/organoid behavior is exemplified in FIG. 6D.

In this regard, reference is also made to the method for training an AI algorithm for determining organoids behavior.

Brain organoids were generated from cells derived from a healthy donor, at least with respect to having PD, and were divided into two groups: a control group of organoids, and a group of organoids that were subjected to a perturbation by competitive antagonist at GABA type A receptors that models/mimics PD (PD-like perturbation).

The two groups (FIG. 6A; two ‘players’), healthy control organoids and PD-like perturbed organoids were cultured in the system for assessment of PD and were subjected to predetermined/‘fixed’ treatment/stimuli.

The processor of the system was further connected to a visualization component presenting a functional cognitive computer simulation/assay that examined the behavior/response of the brain organoid (the two ‘players’) to the predetermined/‘fixed’ treatment/stimuli.

For example, a predetermined treatment/stimuli (presented as coin in the computer game) may include one or more repetitive sessions having same pattern including for example 2 ‘short’ stimulus having high amplitude at the right region of the organoid, followed by a longer stimulus having low amplitude at left region of the organoid.

The processor runs an AI algorithm that was trained on brain-organoids behaviors of a plurality of healthy and/or PD derived brain organoids in response to the predetermined treatment/stimulus described above, and the training data was labeled according to one or more of the predetermined parameters of the treatment/stimulus (e.g., the spatial orientation, amplitude, frequency, duration etc.,).

The AI algorithm repeatedly learned the behavioral response of the healthy control organoids and the PD-like perturbed organoids in response to the functional cognitive assay, i.e., performance of the ‘players’, compared it with the training data, classified it according to its level of similarity, and scored the probability that the ‘player’ is healthy.

Advantageously and surprisingly, as can be seen in FIG. 6A by the end of the game the PD-like perturbed organoids (Right; 3 green squares) was evaluated as having only 60% chance of being healthy as compared to the control organoid which was scored as having 100% chance of being healthy (Left; 5 green squares).

FIG. 6B presents another example of computer simulation/assay that is performed to evaluate cognitive abilities, and can be used as part of both open and closed modes.

Here, a dot movement in 2D space represents an organoid behavior in response to a predetermined stimuli, whereas a dot that moves in a periodically manner through space (i.e., in the same pattern) is classified as ASD-derived, while a dot that moves more randomly through space is classified as healthy.

### Example 6—Assessment of a Learning-Behavior Response Associated with an ASD-Derived Brain Organoid Using a Closed Loop Approach for Assaying Social Interaction Simulation

The following exemplifies the steps of the method for assessing PD related to the analyses of the recorded data, and determining the organoid behavior based on a simulation of the stimuli-response sessions that are visualized as a computer game, and further classifying the behavior (whether PD like behavior or healthy like behavior) or scoring the result as a likelihood for certain severity of PD.

The visual simulation that is presented in FIG. 6C are a theoretical example of how the organoid behavior can be visualized after determining it based on the network response.

A non-limiting example of type of analyses that underly determining the network/organoid behavior is exemplified in FIG. 6D.

In this regard, reference is also made to the method for training an AI algorithm for determining organoids behavior.

The processor is further connected to a visualization component presenting a social interaction computer simulation/assay that examined the capacity of the brain organoid to learn social interaction abilities by determining the learning-behavior of the organoid in response to previous treatments/sessions where a positive or negative feedback treatment was provided, i.e., “elastic” parameters including stimulus spatial pattern, intensity, duration, amplitude, frequency).

The AI algorithm learns/determines the ability of the brain organoid to learn from session to session where a positive or negative feedback treatment was provided and classified it accordingly.

FIG. 6C provides an example of how such a ‘game’ is conducted. A healthy organoid (presented as dots in the computer game) gets closer and closer as they receive positive feedback, while ASD-derived organoids randomly drift in space as they receive negative feedback.

More specifically, during the session the behavior of the ASD-derived brain organoid is simulated using a random movement in a 2D space of two dots, each is simulating the behavior of a different ASD-derived brain organoid generated and obtained from the same subject. FIG. 6C shows that when the dots come closer to each other interaction occurs and a positive feedback treatment is determined/executed, but when the dots move apart from each other and there is no interaction a negative feedback treatment is determined/executed. A learning-behavior response is computed based on the change in the post-treatment behavior of the ASD-derived brain organoid.

The AI algorithm is trained on brain-organoids learning behaviors of a plurality of healthy and/or PD derived brain organoids in response to a positive or negative feedback treatment/stimulus, and the training data is labeled according to one or more changes in parameters of the treatment/stimulus.

Advantageously and surprisingly the simulation demonstrates that by the end of the session the dots of the ASD-derived brain organoid are positioned far apart from each other in comparison to dots representing a simulated behavior of a healthy-brain organoid.

### Example 7—Compound Screening In-Culture and Personalized Medicine

In some embodiments, PD-derived brain organoids are used for compound screening in-culture (e.g., drug discovery) and for personalized evaluation of treatment with a medicament, based on its efficacy in-culture or in-vivo.

Advantageously, for drug screening a plurality of potential compounds are added to a plurality of PD-derived brain organoids in culture, and the effect of the compounds is evaluated by the system for assessing PD, using an open or closed methodology, compared with non-treated PD-derived brain organoids.

Advantageously, for personalized evaluation of treatment efficacy with a psychiatric/neurologic/neurodevelopmental medicament, the medicament is added to PD-derived brain organoids generated from the suffering patient and the efficacy of the medicament is evaluated in culture by the system for assessing PD, using an open or closed methodology, compared with non-treated PD-derived brain organoids.

Furthermore, personalized evaluation of treatment efficacy may be performed after the suffering patient itself was diagnosed (during its prenatal or neonatal stage), and treated (as an adult). Here the treatment may include, for example, but is not necessarily limited to a psychiatric/neurologic/neurodevelopmental medicament. A suffering patient is treated/administered with a therapeutically effective amount of psychiatric/neurologic/neurodevelopmental medicament, PD-derived brain organoids are generated from the suffering patient before and after the treatment with the medicament, and the efficacy of the treatment is evaluated in culture by the system for assessing PD, using an open or closed methodology, by comparing the brain organoids generated from the suffering patient before and after the treatment.

### Example 8: Closed Loop and Open Loop System

In some embodiments, the method underlying the herein disclosed system for assessment of a psychiatric disorder (PD) is based on determining a ‘behavior’ of a brain organoid in response to treatment/stimuli. The ‘behavior’ of the organoid corresponds to its neuronal function/activity in response to the treatment that was provided. The brain organoid may be a PD-derived brain organoid that is subjected to a compound or a medicament in order to determine its ability to affect the severity of PD of the organoid (i.e., evaluation of efficacy of a drug), or it can be an unknown/undetermined brain organoid that is being evaluated for having PD (i.e., diagnosis).

Determination of the brain organoid's behavior includes a comparison thereof with a prediction of behavior of a PD-derived organoid and/or a healthy organoid. The level of similarity to the predicted behavior enables evaluation of its PD severity (i.e., probability) and classification to a category of healthy or PD. As can be seen in FIG. 7A system components, their structural and functional relations include: (1) a brain organoid in 2D/3D culture; (2) a sensor (multi-array electrode (MAE)) coupled to a recorder (recording head stage (RHS)) capable of detecting and recording signals from the brain organoid; (3) a micro-controller unit (MCU) configured to receive, integrate and/or transmit information/data indicative of neuronal function/activity derived from the signals; (4) Optionally FPGA (Field-Programmable Gate Array) or Equivalent Element, before, after or integrated in the MCU, responsible for high-speed parallel signal processing tasks, such as spike detection and classification, as well as the synchronization of stimulation triggers and/or a computer/processor capable of determine an organoid behavior, optionally to simulate it as a computer game through a simulator, give instructions to provide a predetermined treatment (Open loop), or a feedback treatment (Closed loop), and computing an output; (5) optionally, a visualization component such as a screen/monitor/robot, or the like, connected to the computer; (6) a stimuli/manipulation system connected to the computer and capable of delivering treatment to the brain organoid in culture. (7) The system computes an output including computation of the overall behavior responses (Open loop), or a learning behavior responses (Closed loop), for PD, healthy or undetermined brain organoids derived from any developmental stage (e.g., prenatal, mature, mature baby, or adult); and (8) assesses probability of severity of PD based on similarity between the organoids behavior and classify accordingly (9) thereby providing a platform that can be utilized for diagnostics and drug screening and/or as a mean for personalized medicine aiming at evaluation/prediction of clinical success of treatment with a psychiatric drug, including a medicament for neurologic, neurodevelopmental, and neurodegenerative disease

As can be further seen in FIG. 7A, the system and methods provide two types of approaches for assessing severity of PD:

A first approach is the ‘open loop’ approach, which is based on a behavior in response to a predetermined treatment. In this approach the treatment is characterized by having “fixed” parameters, including its spatial pattern, intensity, duration, amplitude, frequency, concentration and/or temperature.

Since according to the ‘open loop’ the stimuli provided to the brain is repeated in a predetermined/fixed manner and does not change from session to session, the AI algorithm, repeatedly learns the behavioral response of the brain (to the functional cognitive assay) and classifies it accordingly. In accordance the AI algorithm is continuously reinforced, based on repetition in the determined brain-organoid behavior, to thereby improve the predicted behavior.

The algorithm is trained on brain-organoids behaviors of a plurality of healthy and/or PD derived brain organoids in response to the predetermined treatment/stimulus, wherein the training data is labeled according to one or more predetermined parameters of the treatment/stimulus.

A second approach is the ‘closed loop’ approach, which is based on a learning-behavior response that consider the change in the behavior (i.e., learning) of a brain organoid in response to positive or negative feedback treatment that is characterized by having “elastic” parameters including its overall spatial pattern, intensity, duration, amplitude, frequency, concentration and/or temperature.

Since according to the ‘closed loop’ the stimuli provided to the brain is a positive or negative feedback treatment that changes from session to session (elastic) based on the learning behavior of the brain organoid in response to previous treatments, the AI algorithm, learns the ability of the brain organoid to learn (functional cognitive assays) and classifies it accordingly. In accordance the AI algorithm is continuously reinforced, based on the determined brain-organoid learning behavior response, to thereby improve the predicted learning behavior.

The AI algorithm is trained on brain-organoids learning behaviors of a plurality of healthy and/or PD derived brain organoids in response to a positive or negative feedback treatment/stimulus, wherein the training data is labeled according to one or more changes in parameters of the treatment/stimulus determined based on the organoids behavior (positive or negative feedback).

In addition, the system is scalable. As can be seen in FIG. 7B the structure is scaled up by including a plurality of each unit, including a plurality of cultures brain organoids, plurality of RHS, plurality of MEA, and plurality of monitors.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1.-51. (canceled)

52. A method for prenatal and/or neonatal assessment of psychiatric disorder (PD), the method comprising:
(i) obtaining human prenatal cells from isolated from an amniotic fluid or neonatal cells;
(ii) generating a prenatal or neonatal derived-brain organoid from the cells; and
(iii) assessing one or more signal(s) and/or attribute(s) of the brain organoid.

53. The method of claim 52, wherein the cells are prenatal cells isolated from an amniotic fluid.

54. The method of claim 52, wherein the assessing comprises determining a degree of similarity between the one or more signal(s) and/or attribute(s) of the prenatal or neonatal brain organoid and PD-derived organoids, or between the one or more signal(s) and/or attribute(s) of the prenatal or neonatal brain organoid and healthy organoids.

55. The method of claim 54, further comprising classifying the prenatal or neonatal organoid as ‘PD’ or ‘healthy’ based on the determined degree of similarity and scoring or classifying the prenatal or neonatal brain organoid according to ‘severity of PD’ based on the determined degree of similarity.

56. The method of claim 52, wherein the one or more signal(s) and/or attribute(s) comprise a visual 3D structure and/or an electrophysiological activity of the organoid.

57. The method of claim 52, wherein the one or more attribute(s) comprises one or more of organoid shape, organoid size, and spines density, or any combination thereof.

58. The method of claim 57, wherein the attribute of organoid shape comprises a deviation in the amount of neural projections of a PD-derived organoid from the amount of neural projections of a healthy organoid; wherein the deviation includes increment or decrease in the amount of sprouting.

59. The method of claim 57, wherein the attribute of organoid size comprises a deviation in a mean organoid diameter (A) of a PD-derived organoid from a mean organoid diameter (A) of a healthy organoid; wherein the deviation includes increment or decrease in the diameter; or a deviation in a ratio between the organoid diameter (A) (μm) and its outwards neural projection (B) (μm) of a PD-derived organoid from a ratio organoid diameter (A) of a healthy organoid; wherein the deviation includes increment or decrease in the ratio.

60. The method of claim 57, wherein assessing the attribute of organoid shape and/or size comprises incubating the organoid with an activator or an inhibitor before determining organoid diameter (A) (μm).

61. The method of claim 57, wherein the attribute of spine density comprises a deviation in an amount of spines along a dendritic projection of a PD-derived organoid from an amount of spines along a dendritic projection of healthy organoid, wherein the deviation includes increment or decrease in the density.

62. The method of claim 52, wherein the one or more signal(s) comprises electrical activity; wherein the electrical activity is indicative of neuronal function; and wherein the electrical activity comprises electrophysiological measurements and/or activity reporter imaging.

63. The method of claim 62, wherein the electrical activity indicative of neuronal function comprises electrical activity signal recorded from the brain organoid in response to one or more stimuli sessions, each session comprising an electrophysiological stimuli provided to the brain organoid.

64. A system for prenatal and/or neonatal assessment of psychiatric disorder (PD), the system comprising:
(i) a prenatal or neonatal-derived brain organoid;
(ii) one or more sensors coupled to a recorder capable of detecting and recording one or more signal(s) and/or attribute(s) of the prenatal or neonatal-derived brain organoid;
(iii) a micro-controller unit (MCU) configured to receive, integrate and/or transmit data of the one or more signal(s) and/or attribute(s);
(iv) a processor configured to:
a. obtain the recorded data of the one or more signal(s) and/or attribute(s);
b. assessing the one or more signal(s) and/or attribute(s) based on the data.

65. The system of claim 64, wherein the computer/processor is further configured to: apply an AI algorithm on the data of the one or more signal(s) and/or attribute(s) to thereby classify the brain organoid based on a degree of similarity of the one or more signal(s) and/or attribute(s) of the organoid to PD-derived organoids or healthy organoids; and wherein the algorithm classifies the organoid according to ‘severity of PD’ or ‘healthy’.

66. The system of claim 64, wherein the one or more signal(s) and/or attribute(s) comprise a visual 3D structure and/or an electrophysiological activity of the organoid; wherein the one or more attribute(s) comprises one or more of organoid shape, organoid size, and spines density, or any combination thereof.

67. The system of claim 64, further comprising a stimuli system capable of delivering stimuli to the prenatal or neonatal brain organoid; and wherein the computer/processor is further configured to send instructions to the stimuli system to provide one or more stimuli sessions, each session comprising an electrophysiological stimuli provided to the brain organoid.

68. The system of claim 64, wherein the computer/processor is further configured to determining a behavior of the brain organoid based on the electrical activity signal recorded in response to the one or more treatment/stimuli session.

69. The system of claim 68, wherein the processor is further configured apply an AI algorithm on the brain organoid behavior to thereby classify the brain organoid based on a degree of similarity of the determined behavior of the brain organoid to a predicted behavior of a PD-derived brain organoids and/or heathy organoids; and wherein the algorithm classifies the organoid according to ‘severity of PD’ or ‘healthy’.

70. The system of claim 68, further comprising a display configured to display a visual simulation representative of the determined organoid behavior.

71. The system of claim 64, wherein the processor is further configured to:
repeatedly trigger stimuli sessions to the organoid,
obtain data recorded in response to each session,
determine a brain-organoid behavior based on the recorded data, after treatment of the organoid with a PD medicament; and
determine an efficacy of the treatment, based on the determined behavior.
